(19) United States
(12) Patent Application Publication
(10) Pub. No.: US 2010/0298381 A1
(43) Pub. Date: Nov. 25, 2010

(54) GAMMA SECRETASE MODULATORS

(75) Inventors: Zhaoning Zhu, Plainsboro, NJ (US); William J. Greenlee, Teaneck, NJ (US); John P. Caldwell, Ringwood, NJ (US); Robert D. Mazzola, JR., Stewartsville, NJ (US); Brian McKittrick, New Vernon, NJ (US); Chad E. Bennett, Metuchen, NJ (US); Duane A. Burnett, Bernardsville, NJ (US)

Correspondence Address: MERCK;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 12/676,035

(22) PCT Filed: Sep. 25, 2008
(86) PCT No.: PCT/US08/11112

§ 371 Date: Jul. 12, 2010

Related U.S. Application Data
(60) Provisional application No. 60/975,959, filed on Sep. 28, 2007.

(51) Int. Cl. A61K 31/454; C07D 403/10; A61K 31/4178; C07D 403/14; A61P 25/28; A61P 27/06; A61P 25/00

(52) U.S. Cl. 514/326; 548/314.4; 514/389; 546/210

(57) ABSTRACT

In its many embodiments, the present invention provides a novel class of 5-membered, nitrogen-containing heterocyclic compounds as modulators of gamma secretase, methods of preparing such compounds, pharmaceutical compositions containing one or more such compounds, methods of preparing pharmaceutical formulations comprising one or more such compounds, and methods of treatment, prevention, inhibition, or amelioration of one or more diseases associated with the central nervous system using such compounds or pharmaceutical compositions.

### REFERENCE TO RELATED APPLICATION

This application claims the benefit of U.S. Provisional Application Ser. No. 60/975,959 filed Sep. 28, 2007.

### FIELD OF THE INVENTION

The present invention relates to certain heterocyclic compounds useful as gamma secretase modulators (including inhibitors, antagonists and the like), pharmaceutical compositions containing the compounds, and methods of treatment using the compounds and compositions to treat various diseases including central nervous system disorders such as, for example, neurodegenerative diseases such as Alzheimer's disease and other diseases relating to the deposition of amyloid protein.

They are especially useful for reducing Amyloid beta (hereinafter referred to as Aβ) production which is effective in the treatment of diseases caused by Aβ such as, for example, Alzheimers and Down Syndrome.

### BACKGROUND OF THE INVENTION

Alzheimer's disease is a disease characterized by degeneration and loss of neurons and also by the formation of senile plaques and neurofibrillary change. Presently, treatment of Alzheimer's disease is limited to symptomatic therapies with a symptom-improving agent represented by an acetylcholinesterase inhibitor, and the basic remedy which prevents progress of the disease has not been developed. A method of controlling the cause of onset of pathologic conditions needs to be developed for creation of the basic remedy of Alzheimer's disease.

Aβ protein, which is a metabolite of amyloid precursor protein (hereinafter referred to as APP), is considered to be greatly involved in degeneration and loss of neurons as well as onset of demential conditions (for example, see Klein W L, et al Proceeding National Academy of Science USA, Sep. 2, 2003, 100(18), p. 10417-22, suggest a molecular basis for reversible memory loss.

Nitsch R M, and 16 others, Antibodies against β-amyloid slow cognitive decline in Alzheimer's disease, Neuron, May 22, 2003, 38(4), p. 547-554) suggest that the main components of Aβ protein are Aβ340 consisting of 40 amino acids and Aβ42 having two additional amino acids at the C-terminal. The Aβ40 and Aβ42 tend to aggregate (for example, see Jarrell J T et al, The carboxy terminus of the β amyloid protein is critical for the seeding of amyloid formation: implications for the pathogenesis of Alzheimer's disease, Biochemistry, May 11, 1993, 32(18), p. 4693-4697) and constitute main components of senile plaques (for example, (Glenner G G, et al, Alzheimer's disease: initial report of the purification and characterization of a novel cerebrovascular amyloid protein, Biochemical and Biophysical Research Communications, May 16, 1984, 120(3), p. 885-90. See also Masters C L, et al, Amyloid plaque core protein in Alzheimer disease and Down syndrome, Proceeding National Academy of Science USA, June 1985, 82(12), p. 4245-4249.).

Furthermore, it is known that mutations of APP and presenelin genes, which is observed in familial Alzheimer's disease, increase production of Aβ40 and Aβ42 (for example, see Gouras G K, et al, Intraneuronal A/β142 accumulation in human brain, American Journal of Pathology, January 2000, 156(1), p. 15-20. Also, see Scheuner D, et al, Nature Medicine, August 1996, 2(8), p. 864-870; and Forman M S, et al, Differential effects of the Swedish mutant amyloid precursor protein on β-amyloid accumulation and secretion in neurons and normeuronal cells, Journal of Biological Chemistry, Dec. 19, 1997, 272(51), p. 32247-32253.). Therefore, compounds which reduce production of Aβ40 and Aβ542 are expected as an agent for controlling progress of Alzheimer's disease or for preventing the disease.

These Aβs are produced when APP is cleaved by beta secretase and subsequently clipped by gamma secretase. In consideration of this, creation of inhibitors of γ secretase and β secretase has been attempted for the purpose of reducing production of Aβs. Many of these secretase inhibitors already known are peptides or peptidomimetics such as L-685,458. L-685,458, an aspartyl protease transition stale mimic, is a potent inhibitor of amyloid β-protein precursor γ-secretase activity, Biochemistry, Aug. 1, 2000, 39(30), p. 8698-8704).

Also of interest in connection with the present invention are: US 2007/0117798 (Eisai, published May 24, 2007); US 2007/0117839 (Eisai, published May 24, 2007); US 2006/0004013 (Eisai, published Jan. 5, 2006); WO 2005/110422 (Boehringer Ingelheim, published Nov. 24, 2005); WO 2006/045554 (Cellzone AG, published May 4, 2006); WO 2004/110350 (Neurogenetics™, published Dec. 23, 2004); WO 2004/071431 (Myriad Genetics, published Aug. 26, 2004); US 2005/0042284 (Myriad Genetics, published Feb. 23, 2005) and WO 2006/001877 (Myriad Genetics, published Jan. 5, 2006).

There is a need for new compounds, formulations, treatments and therapies to treat diseases and disorders associated with Aβ. It is, therefore, an object of this invention to provide compounds useful in the treatment or prevention or amelioration of such diseases and disorders.

### SUMMARY OF THE INVENTION

In its many embodiments, the present invention provides a novel class of heterocyclic compounds as gamma secretase modulators (including inhibitors, antagonists and the like), methods of preparing such compounds, pharmaceutical compositions comprising one or more such compounds, methods of preparing pharmaceutical formulations comprising one or more such compounds, and methods of treatment, prevention, inhibition or amelioration of one or more diseases associated with the Aβ using such compounds or pharmaceutical compositions.

In another embodiment, the present application discloses a compound, or pharmaceutically acceptable salts, solvates, esters or prodrugs of said compound, said compound having the general structure shown in Formula (I):

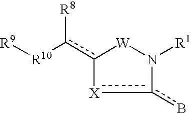

wherein R¹, R⁸, R⁹, R¹⁰, B, W and X are independently selected and are as defined below.

This invention provides compounds of formula (I).

This invention also provides pharmaceutically acceptable salts, esters and solvates of the compounds of formula (I).

This invention also provides compounds of formula (I) in pure and isolated form.

This invention also provides compounds of formulas IA to IM. This invention also provides compounds of formulas A9a1 to A9k1, A9n1 to Aq1, A9a to A9u, A9ab, B1-B11, (+)-B11, (−)-B11, B12-B23, C7a to C7f, and D1.

This invention also provides a pharmaceutical composition comprising an effective amount of one or more (e.g., one) compounds of formula (I), or a pharmaceutically acceptable salt, solvate, or ester thereof, and a pharmaceutically acceptable carrier.

This invention also provides a pharmaceutical composition comprising an effective amount of one or more (e.g., one) compounds of formula (I), an effective amount of one or more (e.g., one) other pharmaceutically active ingredients (e.g., drugs) as described below for example, and a pharmaceutically acceptable carrier.

The compounds of Formula (I) can be useful as gamma secretase modulators and can be useful in the treatment and prevention of diseases such as, for example, central nervous system disorders such as Alzheimers disease and Downs Syndrome.

Thus, this invention also provides methods for: (1) method for modulating (including inhibiting, antagonizing and the like) gamma-secretase; (2) treating one or more neurodegenerative diseases; (3) inhibiting the deposition of amyloid protein (e.g., amyloid beta protein) in, on or around neurological tissue (e.g., the brain); (4) Alzheimer's disease; and (5) treating Downs syndrome; wherein each method comprises administering an effective amount of one or more (e.g., one) compounds of formula (I) to a patient in need of such treatment.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of one or more (e.g. one) compounds of formula (I) and the administration of one or more (e.g., one) other pharmaceutical active ingredients (e.g., drugs). The compounds of formula (I) and the other drugs can be administered separately (i.e., each is in its own separate dosage form), or the compounds of formula (I) can be combined with the other drugs in the same dosage form.

This invention also provides methods for: (1) treating mild cognitive impairment; (2) treating glaucoma; (3) treating cerebral amyloid angiopathy; (4) treating stroke; (5) treating dementia; (6) treating microgliosis; (7) treating brain inflammation; and (8) treating olfactory function loss; wherein each method comprises administering an effective amount of one or more (e.g., one) compounds of formula (I) to a patient in need of such treatment.

This invention also provides a kit comprising, in separate containers, in a single package, pharmaceutical compositions for use in combination, wherein one container comprises an effective amount of a compound of formula (I) in a pharmaceutically acceptable carrier, and another container (i.e., a second container) comprises an effective amount of another pharmaceutically active ingredient (as described below), the combined quantities of the compound of formula (I) and the other pharmaceutically active ingredient being effective to treat the diseases or conditions mentioned in any of the above methods.

This invention also provides any one of the above mentioned methods of treatment wherein the compound of formula (I) is selected from the group consisting of the compounds in the ILLUSTRATIVE EXAMPLES.

### DETAILED DESCRIPTION

In another embodiment, the present application discloses a compound, or pharmaceutically acceptable salts, solvates, esters or prodrugs of said compound, said compound having the general structure shown in Formula (I):

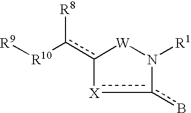

wherein:

R¹, R⁸, R⁹, R¹⁰, B, W and X are independently selected;

B is

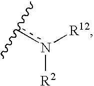

H, alkoxy, alkyl, cycloalkyl, alkoxyalkyl, hydroxyalkyl, heterocycloalkyl, ═N—O-alkyl, —OR^{15a}, ═O or ═S, provided than when X is —N(R¹⁴)— or ═N—, and W is —C(O)—, B is not ═O or ═S;

W is —C(O)— or —S(O)₂—;

X is —N(R¹⁴)— or —C(R⁶)(R⁷)— (and those skilled in the art will appreciate that when the optional bond to X is present then X is —N, or —C(R⁶)═, or —C(R⁷)═);

when B is

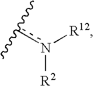

each dashed line of

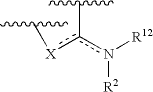

represents an optional bond with the proviso that only one optional bond (- - -) is present, and when the optional bond between the nitrogen of N(R²)(R¹²) and the adjacent ring carbon is present, then R¹² is absent (i.e. B is ═N—R²);

the dashed line of

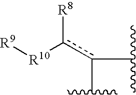

represents an optional bond, and when the optional bond is absent, the moiety comprising R⁸, R⁹ and R¹⁰ is selected from the group consisting of:

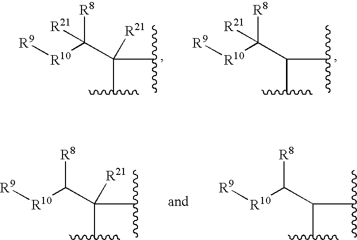

wherein each R²¹ is independently selected; and when the optional bond is present, the moiety comprising R⁸, R⁹ and R¹⁰ is

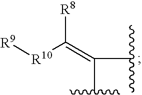

each dashed line of

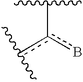

is an optional bond, with the proviso that one optional bond (- - -) is present at any given time;

R¹ is selected from the group consisting of H, alkyl-, alkenyl-, alkynyl-, aryl-, arylalkyl-, alkylaryl-, cycloalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of the moieties shown below;

R² is selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶ and —P(O)(OR¹⁵)(OR¹⁶), and wherein each of the alkyl, cycloalkyl, cycloalkylalkyl, cycloalkenyl, heterocyclyl, heterocyclylalkyl, aryl, arylalkyl, heteroaryl, heteroarylalkyl, alkenyl and alkynyl groups are independently unsubstituted or substituted by 1 to 5 R²¹ groups;

R¹² is independently selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁶)R¹⁶ and —P(O)(OR¹⁵)(OR¹⁶), and wherein each of the alkyl, cycloalkyl, cycloalkylalkyl, cycloalkenyl, heterocyclyl, heterocyclylalkyl, aryl, arylalkyl, heteroaryl, heteroarylalkyl, alkenyl and alkynyl groups are independently unsubstituted or substituted by 1 to 5 R²¹ groups;

each R¹⁴ be the same or different, each being independently selected from the group consisting of H, alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), S(O)₂N(R¹⁵)(R¹⁶) —S(O)R¹⁵, —S(O)₂R¹⁵, —C(═NOR¹⁵)R¹⁶, and —P(O)(OR¹⁵)(OR¹⁶); and wherein each of the alkyl, cycloalkyl, cycloalkylalkyl, cycloalkenyl, heterocyclyl, heterocyclylalkyl, aryl, arylalkyl, heteroaryl, heteroarylalkyl, alkenyl and alkynyl groups in are independently unsubstituted or substituted by 1 to 5 R²¹ group;

R⁶ is selected from the group consisting of H, halo, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁷ is selected from the group consisting of H, halo, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- can be unsubstituted or optionally independently substituted with 1-5 substituents which can be the same or different, each substituent being independently selected from the group consisting of consisting of the moieties shown below;

R⁸ is selected from the group consisting of H, halo, alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, with each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl- being unsubstituted or optionally independently substituted with 1-3 substituents which can be the same or different, each substituent being independently selected from the group consisting of the moieties shown below;

R⁹ is selected from the group consisting of alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-, wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- and heterocyclyalkyl-can be unsubstituted or optionally independently substituted with 1-3 substituents which can be the same or different, each substituent being independently selected from the group consisting of the moieties shown below,

R¹⁰ is selected from the group consisting of a bond, alkyl-, alkenyl-, alkynyl-, aryl-, arylalkyl-, alkylaryl-, cycloalkyl-, cycloalkylalkyl-, heteroaryl-, heteroarylalkyl-, heterocyclyl-, heterocyclyalkyl- and the moieties:

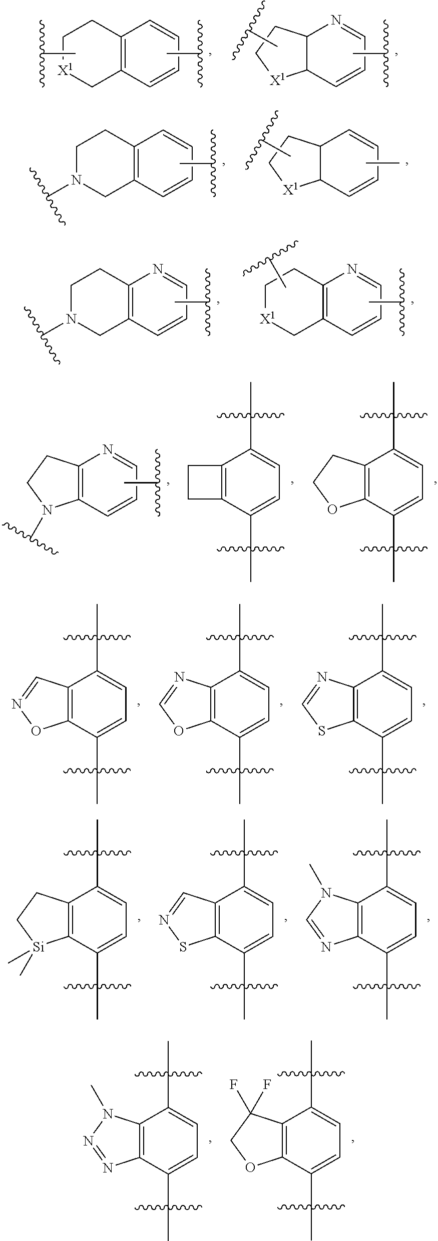

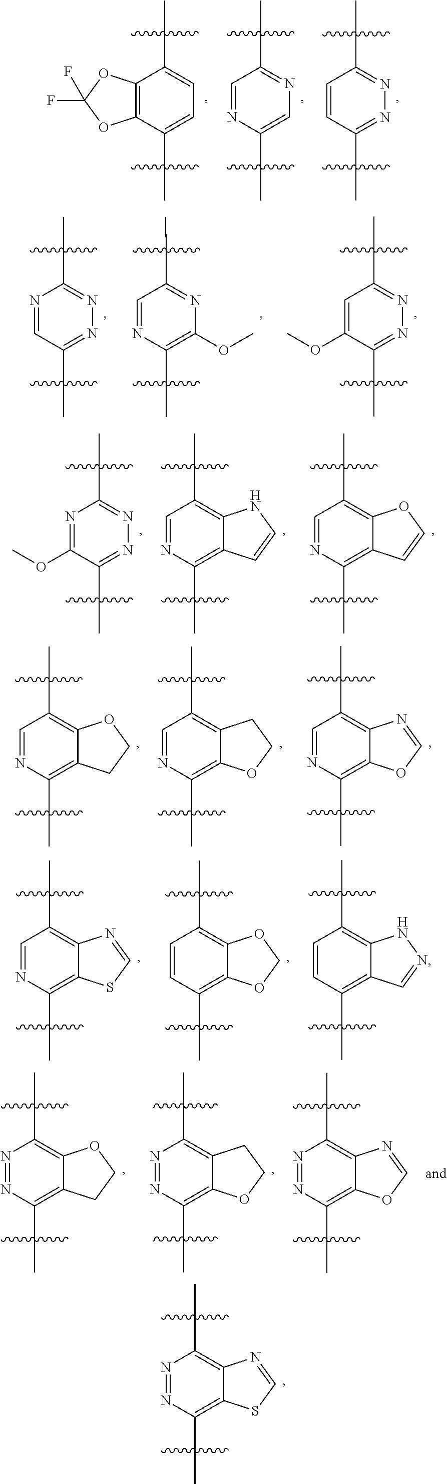

where X¹ is O, N(R¹⁴) or S;

wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- heterocyclyalkyl- and the above-noted moieties for R¹⁰ can be unsubstituted or optionally independently substituted with 1-3 substituents which can be the same or different, each being independently selected from the group consisting of the moieties shown below; and

R^{15a} is independently selected from the group consisting of alkyl, cycloalkyl, cycloalkylalkyl, heterocyclyl, heterocyclylalkyl, arylalkyl, heteroarylalkyl, arylcycloalkyl, arylheterocyclyl, R¹⁸-alkyl, R¹⁸-cycloalkyl, R¹⁸-cycloalkylalkyl, R¹⁸-heterocyclyl, R¹⁸-heterocyclylalkyl, R¹⁸-aryl, R¹⁸-arylalkyl, R¹⁵-heteroaryl and R¹⁸-heteroarylalkyl;

R¹⁵ is independently selected from the group consisting of H, alkyl, cycloalkyl, cycloalkylalkyl, heterocyclyl, heterocyclylalkyl, arylalkyl, heteroarylalkyl, arylcycloalkyl, arylheterocyclyl, R¹⁸-alkyl, R¹⁸-cycloalkyl, R¹⁸-cycloalkylalkyl, R¹⁸-heterocyclyl, R¹⁸-heterocyclylalkyl, R¹⁸-aryl, R¹⁸-arylalkyl, R¹⁸-heteroaryl and R¹⁸-heteroarylalkyl;

R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, R¹⁸-alkyl, R¹⁸-cycloalkyl, R¹⁸-cycloalkylalkyl, R¹⁸-heterocyclyl, R¹⁸-heterocyclylalkyl, R¹⁸-aryl, R¹⁸-arylalkyl, R¹⁸-heteroaryl and R¹⁸-heteroarylalkyl;

R¹⁸ is 1-5 substituents independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁹, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

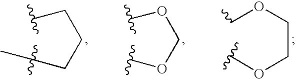

R¹⁹ is alkyl, cycloalkyl, aryl, arylalkyl or heteroarylalkyl;

R²⁰ is alkyl, cycloalkyl, aryl, halo substituted aryl, arylalkyl, heteroaryl or heteroarylalkyl;

wherein each of the alkyl, cycloalkyl, cycloalkylalkyl, cycloalkenyl, heterocyclyl, heterocyclylalkyl, aryl, arylalkyl, alkylaryl, heteroaryl, heteroarylalkyl, alkenyl and alkynyl groups in R¹, R², R⁶, R⁷, R⁸, R⁹, R¹⁰, R¹² and R¹⁴, are independently unsubstituted or substituted by 1 to 5 R²¹ groups independently selected from the group consisting of alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, halo, —CN, —OR¹⁵, —C(O)R¹⁵, —C(O)OR¹⁵, —C(O)N(R¹⁵)(R¹⁶), —SF₅, −OSF₅, —Si(R¹⁵)₃ wherein each R¹⁵ is independently selected, —SR¹⁵, —S(O)N(R¹⁵)(R¹⁶), —CH(R¹⁶)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵; and

wherein each of the alkyl, cycloalkenyl, cycloalkyl, cycloalkylalkyl, heterocyclyl, heterocyclylalkyl, aryl, arylalkyl, heteroaryl, heteroarylalkyl, alkenyl and alkynyl groups in R²¹ are independently unsubstituted or substituted by 1 to 5 R²² groups independently selected from the group consisting of alkyl, cycloalkyl, cycloalkenyl, heterocyclyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SF₅, —OSF₅, —Si(R¹⁵)₃ wherein each R¹⁵ is independently selected, —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

It should be understood that any ring moiety, herein described, independently may optionally additionally be fused with an aryl or heteroaryl ring, wherein the ring moiety resulting from the fusion may be unsubstituted or optionally independently substituted with 1-5 substituents which can be the same or different, each substituent being independently selected from the group consisting of the R²¹ moieties shown above.

In one embodiment of this invention B is

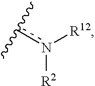

OR^{15a}, ═O or ═S, provided than when X is —N(R¹⁴)— and W is —C(O)—, B is not ═O or ═S.

In another embodiment R¹⁰ is selected from the group consisting of a bond, alkyl-, alkenyl-, alkynyl-, aryl-, arylalkyl-, alkylaryl-, cycloalkyl-, cycloalkylalkyl-, heteroaryl-, heteroarylalkyl-, heterocyclyl-, heterocyclyalkyl- and the moieties:

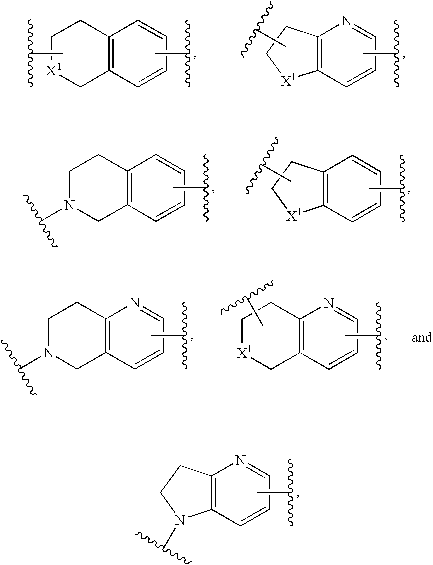

where X¹ is O, N(R¹⁴) or S;

wherein each of said alkyl-, alkenyl-, alkynyl-, aryl-, arylalkyl-, alkylaryl-, cycloalkyl-, cycloalkylalkyl-, heteroaryl-, heteroarylalkyl-, heterocyclyl- heterocyclyalkyl- and the above-noted moieties for R¹⁰ can be unsubstituted or optionally independently substituted with 1-3 R²¹ substituents which can be the same or different, each being independently selected from the group consisting of the moieties shown below.

In another embodiment each of the alkyl, cycloalkyl, cycloalkylalkyl, cycloalkenyl, heterocyclyl, heterocyclylalkyl, aryl, arylalkyl, alkylaryl, heteroaryl, heteroarylalkyl, alkenyl and alkynyl groups in R¹, R², R⁶, R⁷, R⁸, R⁹, R¹⁰, R¹² and R¹⁴, are independently unsubstituted or substituted by 1 to 5 R²¹ groups independently selected from the group consisting of alkyl, alkenyl, alkynyl, cycloalkyl, cycloalkylalkyl, cycloalkenyl, heterocyclyl, heterocyclylalkyl, aryl, arylalkyl, heteroaryl, heteroarylalkyl, halo, —CN, —OR¹⁶, —C(O)R¹⁵, —C(O)OR¹⁵, —C(O)N(R¹⁶)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵; and wherein each of the alkyl, cycloalkenyl, cycloalkyl, cycloalkylalkyl, heterocyclyl, heterocyclylalkyl, aryl, arylalkyl, heteroaryl, heteroarylalkyl, alkenyl and alkynyl groups in R²¹ are independently unsubstituted or substituted by 1 to 5 R²² groups independently selected from the group consisting of alkyl, cycloalkyl, cycloalkenyl, heterocyclyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

Thus, one embodiment of the present invention is directed to a compound of formula (I):

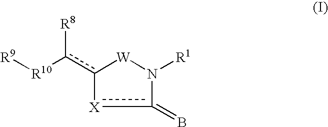

or a pharmaceutically acceptable salt, solvate, or ester thereof, wherein:

R¹, R⁸, R⁹, R¹⁰, B, W and X are independently selected;

the dashed lines (- - -) represent optional bonds, provided that either the optional bond to X is present, or the optional bond to B is present, but not both (i.e., the compound of formula (I) is either:

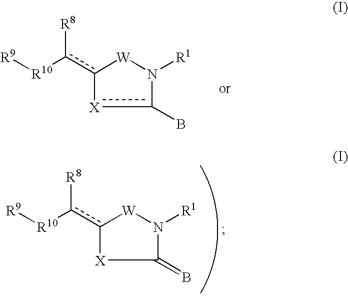

B is selected from the group consisting of: H, alkoxy, alkyl, cycloalkyl, heterocycloalkyl, alkoxyalkyl-, hydroxyalkyl-, —OR^{15a}, ═O, ═S, ═N—O-alkyl, and

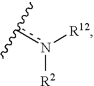

provided that:
(a) when the optional bond to N is present (i.e., the optional bond to B is present) then the R¹² substituent is absent (i.e., the B moiety [symbol] NR²R¹² is —NR²R¹² or ═NR²), and
(b) provided that when X is —N(R¹⁴)— or ═N—, and W is —C(O)—, then B is not ═O or ═S;

W is selected from the group consisting of: —C(O)— and —S(O)₂—;

X is selected from the group consisting of:
(a) —N(R¹⁴)— and —C(R⁶)(R⁷)— when the optional bond to X is present, and
(b) —N═, —C(R⁶)═, and —C(R⁷)═ when the optional bond to X is absent; when the optional bond in the moiety:

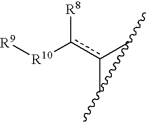

is present then said moiety is:

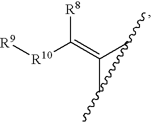

and
when the optional bond in the moiety:

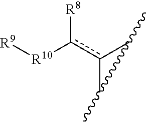

is absent then said moiety is selected from the group consisting of:

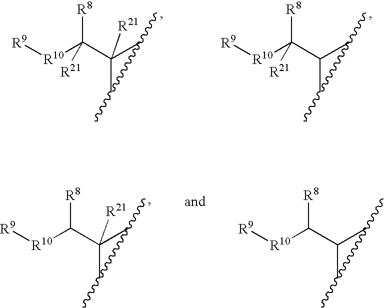

wherein each R²¹ is independently selected (and in one embodiment the moiety

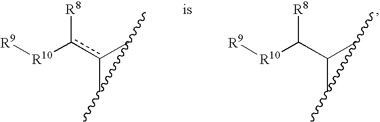

and in another embodiment the moiety

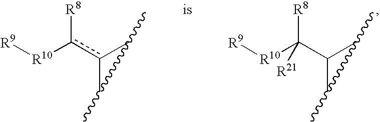

and in another embodiment the moiety

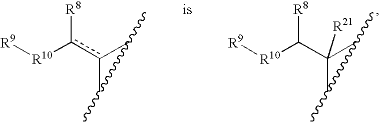

and in another embodiment the moiety

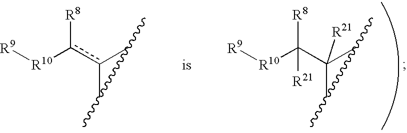

R¹ is selected from the group consisting of H, alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl-, and wherein each of said alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl- R¹ groups are optionally substituted with 1-5 independently selected R²¹ substituents;

R² is selected from the group consisting of H, alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, —CN, —C(O)R¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶ and —P(O)(OR¹⁵)(OR¹⁶), and wherein each of said alkyl, alkenyl, alkynyl, cycloalkyl, cycloalkylalkyl-, cycloalkenyl, heterocyclyl, heterocyclylalkyl-, aryl, arylalkyl-, heteroaryl, and heteroarylalkyl- R² groups are optionally substituted with 1-5 independently selected R²¹ substituents;

R⁶ is selected from the group consisting of H, halo, alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl-, wherein each of said alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl- R⁶ groups are optionally substituted with 1-5 independently selected R²¹ substituents;

R⁷ is selected from the group consisting of H, halo, alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl-, wherein each of said alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl- R⁷ groups are optionally substituted with 1-5 independently selected R²¹ substituents;

R⁸ is selected from the group consisting of H, halo, alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl-, with each of said alkyl, alkenyl, alkynyl, aryl, arylalkyl-, alkylaryl-, cycloalkyl, cycloalkylalkyl-, heteroaryl- heteroarylalkyl-, heterocyclyl and heterocyclyalkyl- R⁸ groups are optionally substituted with 1-3 independently selected R²¹ substituents;

R⁹ is selected from the group consisting of alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl-, wherein each of said alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl- R⁶ groups are optionally substituted with 1-3 independently selected R²¹ substituents;

R¹⁰ is selected from the group consisting of a bond, alkyl, alkenyl, alkynyl, aryl, arylalkyl-, alkylaryl-, cycloalkyl, cycloalkylalkyl-, heteroaryl, heteroarylalkyl-, heterocyclyl, heterocyclyalkyl-,

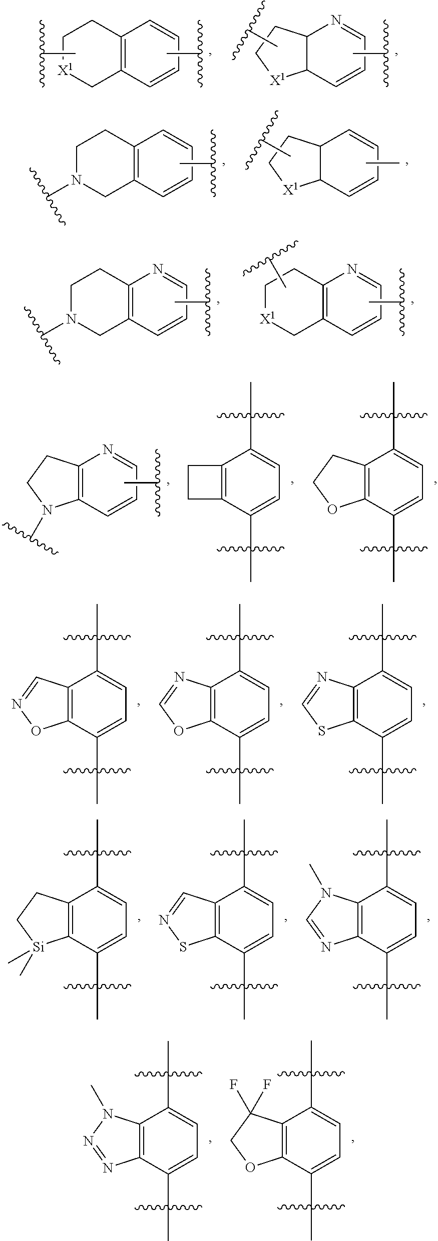

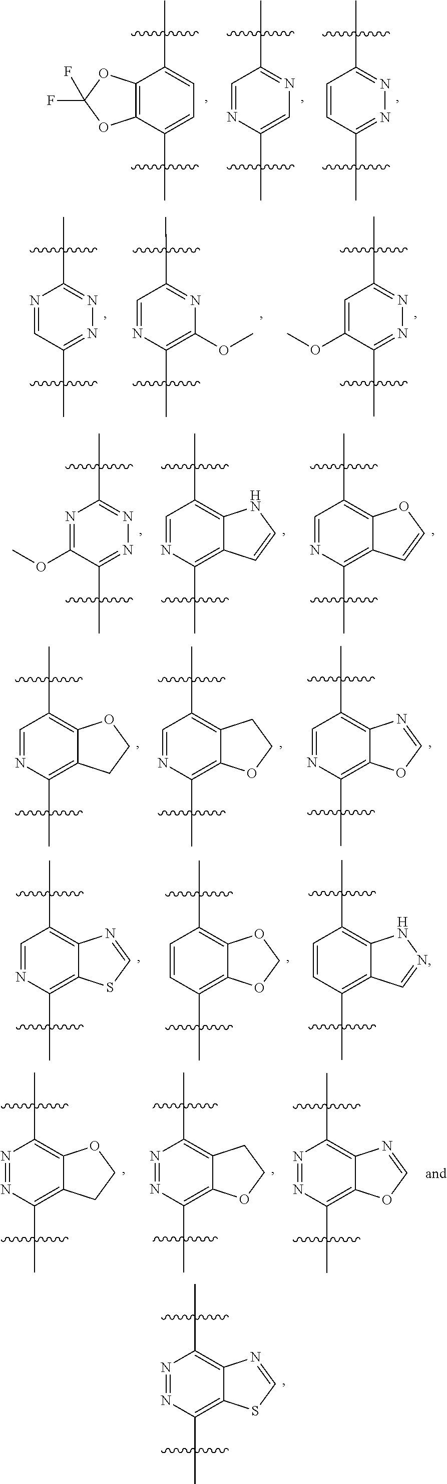

wherein X¹ is O, N(R¹⁴) or S;
   wherein each of said R¹⁰ substituents (excluding the R¹⁰ bond) is optionally substituted with 1-3 independently selected R²¹ substituents;

R¹² is independently selected from the group consisting of H, alkyl, alkenyl, alkynyl, cycloalkyl, cycloalkylalkyl, cycloalkenyl, heterocyclyl, heterocyclylalkyl-, aryl, arylalkyl-, heteroaryl, heteroarylalkyl-, —CN, —C(O)R¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶ and —P(O)(OR¹⁵)(OR¹⁶), and wherein each of said alkyl, alkenyl, alkynyl, cycloalkyl, cycloalkylalkyl-, cycloalkenyl, heterocyclyl, heterocyclylalkyl-, aryl, arylalkyl-, heteroaryl, and heteroarylalkyl- R¹² groups are optionally substituted with 1-5 independently selected R²¹ substituents;

each R¹⁴ be the same or different, each being independently selected from the group consisting of H, alkyl, alkenyl, alkynyl, cycloalkyl, cycloalkylalkyl-, cycloalkenyl, heterocyclyl, heterocyclylalkyl-, aryl, arylalkyl, heteroaryl, heteroarylalkyl-, —CN, —C(O)R¹⁵, —C(O)OR¹⁶, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶, and —P(O)(OR¹⁶)(OR¹⁶), and wherein each of said alkyl, alkenyl, alkynyl, cycloalkyl, cycloalkylalkyl-, cycloalkenyl, heterocyclyl, heterocyclylalkyl-, aryl, arylalkyl-, heteroaryl, and heteroarylalkyl- R¹⁴ groups are optionally substituted with 1-5 independently selected R²¹ substituents;

R^{15a} is independently selected from the group consisting of alkyl, cycloalkyl, cycloalkylalkyl-, heterocyclyl, heterocyclylalkyl-, arylalkyl-, heteroarylalkyl-, arylcycloalkyl-, arylheterocyclyl-, (R¹⁸)ₙ-alkyl-, (R¹⁸)ₙ-cycloalkyl-, (R¹⁸)ₙ-cycloalkylalkyl-, (R¹⁸)ₙ-heterocyclyl-, (R¹⁸)ₙ-heterocyclylalkyl-, (R¹⁸)ₙ-aryl-, (R¹⁸)ₙ-arylalkyl-, (R¹⁸)ₙ-heteroaryl- and (R¹⁸)ₙ-heteroarylalkyl-, wherein n is 1 to 5;

R¹⁵ is independently selected from the group consisting of H, alkyl, cycloalkyl, cycloalkylalkyl-, heterocyclyl, heterocyclylalkyl-, arylalkyl-, heteroarylalkyl-, arylcycloalkyl-, arylheterocyclyl-, (R¹⁸)ₙ-alkyl-, (R¹⁸)ₙ-cycloalkyl-, (R¹⁸)ₙ-cycloalkylalkyl-, (R¹⁸)ₙ-heterocyclyl-, (R¹⁸)ₙ-heterocyclylalkyl-, (R¹⁸)ₙ-aryl-, (R¹⁸)ₙ-arylalkyl-, (R¹⁸)ₙ-heteroaryl- and (R¹⁸)ₙ-heteroarylalkyl-, wherein n is 1 to 5;

R¹⁶ and R¹⁷ are independently selected from the group consisting of H, alkyl, alkenyl, alkynyl, cycloalkyl, cycloalkylalkyl-, heterocyclyl, heterocyclylalkyl-, aryl, arylalkyl-, heteroaryl, heteroarylalkyl-, arylcycloalkyl-, arylheterocyclyl, (R¹⁸)ₙ-alkyl-, (R¹⁸)ₙ-cycloalkyl-, (R¹⁸)ₙ-cycloalkylalkyl-, (R¹⁸)ₙ-heterocyclyl-, (R¹⁸)ₙ-heterocyclylalkyl-, (R¹⁸)ₙ-aryl-, (R¹⁸)ₙ-arylalkyl-, (R¹⁸)ₙ-heteroaryl- and (R¹⁸)ₙ-heteroarylalkyl-;

Each R¹⁸ is independently selected from the group consisting of alkyl, alkenyl, alkynyl, aryl, arylalkyl-, arylalkenyl-, arylalkynyl-, —NO₂, halo, heteroaryl, HO-alkyoxyalkyl, —CF₃, —CN, alkyl-CN, —C(O)R¹⁶, —C(O)OH, —C(O)OR¹⁶, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁸, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁸, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰ , —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);

or, alternately, two R¹⁸ moieties on adjacent carbons can be linked together to form:

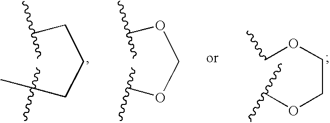

R¹⁹ is selected from the group consisting of: alkyl, cycloalkyl, aryl, arylalkyl- and heteroarylalkyl-;

R²⁰ is selected from the group consisting of: alkyl, cycloalkyl, aryl, halo substituted aryl, arylalkyl-, heteroaryl or heteroarylalkyl-;

Each R²¹ is independently selected from the group consisting of alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, halo, —CN, —OR¹⁵, —C(O)R¹⁵, —C(O)OR¹⁵, —C(O)N(R¹⁵)(R¹⁶), —SF₅, —OSF₅, —Si(R¹⁵)₃ wherein each R¹⁵ is independently selected, —SR¹⁵, —S(O)N(R¹⁵)(R¹⁶), —CH(R¹⁵)(R¹⁶), —S(O)₃N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵; and

wherein each of the alkyl, cycloalkenyl, cycloalkyl, cycloalkylalkyl-, heterocyclyl, heterocyclylalkyl-, aryl, arylalkyl, heteroaryl, heteroarylalkyl-, alkenyl and alkynyl groups in R²¹ are optionally substituted by 1 to 5 R²² groups independently selected from the group consisting of alkyl, cycloalkyl, cycloalkenyl, heterocyclyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SF₅, —OSF₅, —Si(R¹⁵)₃ wherein each R¹⁵ is independently selected, —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

In one embodiment of this invention R¹⁰ is selected from the group consisting of a bond, alkyl, alkenyl, alkynyl, aryl, arylalkyl-, alkylaryl-, cycloalkyl, cycloalkylalkyl-, heteroaryl, heteroarylalkyl-, heterocyclyl, heterocyclyalkyl-,

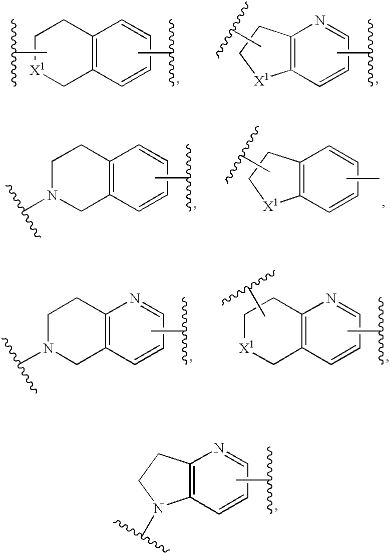

wherein X¹ is O, N(R¹⁴) or S;
   wherein each of said R¹⁰ substituents (excluding the R¹⁰ bond) are optionally substituted with 1-3 independently selected R²¹ substituents.

In one embodiment each R²¹ is independently selected from the group consisting of alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, halo, —CN, —OR¹⁵, —C(O)R¹⁵, —C(O)OR¹⁵, —C(O)N(R¹⁵)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂ and —S(O)₂R¹⁵; and wherein each of the alkyl, cycloalkenyl, cycloalkyl, cycloalkylalkyl-, heterocyclyl, heterocyclylalkyl-, aryl, arylalkyl, heteroaryl, heteroarylalkyl-, alkenyl and alkynyl groups in R²¹ are optionally substituted by 1 to 5 R²² groups independently selected from the group consisting of alkyl, cycloalkyl, cycloalkenyl, heterocyclyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵.

It should be understood that independently each ring moiety substituent in formula (I) can be optionally fused with an aryl or heteroaryl ring, wherein the ring moiety resulting from the fusion can be optionally substituted with 1-5 independently selected R²¹ substituents.

In another embodiment of this invention, there are 1 to 5 R²¹ groups present in formula (I), and at least one (e.g., 1 to 2) R²¹ is selected from the group consisting of: —SF₅, —OSF₅ and —Si(R¹⁵)₃, wherein each R¹⁵ is independently selected.

In another embodiment of this invention, there are 1 to 5 R²¹ groups present in formula (I), and at least one R²¹ is selected from the group consisting of: —SF₅ and —Si(R¹⁵)₃, and each R¹⁵ is the same or different alkyl group.

In another embodiment of this invention, there are 1 to 5 R²¹ groups present in formula (I), and at least one R²¹ is selected from the group consisting of: —SF₅, —OSF₅ and —Si(CH₃)₃.

In another embodiment of this invention, there are 1 to 5 R²¹ groups present in formula (I), and one of the R²¹ groups is selected from the group consisting of: —SF₅, OSF₅ and —Si(R¹⁵)₃.

In another embodiment of this invention, there are 1 to 5 R²¹ groups present in formula (I), and one of the R²¹ groups is selected from the group consisting of: —SF₅, OSF₅ and —Si(R¹⁵)₃, and each R¹⁵ is the same or different alkyl group.

In another embodiment of this invention, there are 1 to 5 R²¹ groups present in formula (I), and one of the R²¹ groups is selected from the group consisting of: —SF₅, —OSF₅ and —Si(CH₃)₃.

In another embodiment of this invention, there are 2 to 5 R²¹ groups present in formula (I), and two of the R²¹ groups are selected from the group consisting of: —SF₅, OSF₅ and —Si(R¹⁵)₃, wherein each R¹⁵ is independently selected.

In another embodiment of this invention, there are 2 to 5 R²¹ groups present in formula (I), and two of the R²¹ groups are selected from the group consisting of: —SF₅, OSF₅ and —Si(R¹⁵)₃, and each R¹⁵ is the same or different alkyl group.

In another embodiment of this invention, there are 2 to 5 R²¹ groups present in formula (I), and two of the R²¹ groups are selected from the group consisting of: —SF₅, —OSF₅ and —Si(CH₃)₃.

In another embodiment of this invention, there are 1 to 5 R²¹ groups present in formula (I), and at least one (e.g., 1 to 2) R²¹ is selected from the group consisting of: —SF₅ and —Si(R¹⁵)₃, wherein each R¹⁵ is independently selected.

In another embodiment of this invention, there are 1 to 5 R²¹ groups present in formula (I), and at least one R²¹ is selected from the group consisting of: —SF₅ and —Si(R¹⁵)₃, and each R¹⁵ is the same or different alkyl group.

In another embodiment of this invention, there are 1 to 5 R²¹ groups present in formula (I), and at least one R²¹ is selected from the group consisting of: —SF₅ and —Si(CH₃)₃.

In another embodiment of this invention, there are 1 to 5 R²¹ groups present in formula (I), and one of the R²¹ groups is selected from the group consisting of: —SF₅ and —Si(R¹⁵)₃.

In another embodiment of this invention, there are 1 to 5 R²¹ groups present in formula (I), and one of the R²¹ groups is selected from the group consisting of: —SF₅ and —Si(R¹⁵)₃, and each R¹⁵ is the same or different alkyl group.

In another embodiment of this invention, there are 1 to 5 R²¹ groups present in formula (I), and one of the R²¹ groups is selected from the group consisting of: —SF₅ and —Si(CH₃)₃.

In another embodiment of this invention, there are 2 to 5 R²¹ groups present in formula (I), and two of the R²¹ groups are selected from the group consisting of: —SF₅ and —Si(R¹⁵)₃, wherein each R¹⁵ is independently selected.

In another embodiment of this invention, there are 2 to 5 R²¹ groups present in formula (I), and two of the R²¹ groups are selected from the group consisting of: —SF₅ and —Si(R¹⁵)₃, and each R¹⁵ is the same or different alkyl group.

In another embodiment of this invention, there are 2 to 5 R²¹ groups present in formula (I), and two of the R²¹ groups are selected from the group consisting of: —SF₅ and —Si(CH₃)₃.

In another embodiment of this invention, there are 1 to 5 R²¹ groups present in formula (I), and one of the R²¹ groups is —SF₅.

In another embodiment of this invention, there are 2 to 5 R²¹ groups present in formula (I), and two of the R²¹ groups are —SF₅.

In another embodiment of this invention, there are 1 to 5 R²¹ groups present in formula (I), and one of the R²¹ groups is —OSF₅.

In another embodiment of this invention, there are 2 to 5 R²¹ groups present in formula (I), and two of the R²¹ groups are —OSF₅.

In another embodiment of this invention, there are 1 to 5 R²¹ groups present in formula (I), and one of the R²¹ groups is —Si(R¹⁵)₃.

In another embodiment of this invention, there are 1 to 5 R²¹ groups present in formula (I), and one of the R²¹ groups is —Si(R¹⁵)₃ and each R¹⁵ is the same or different alkyl group.

In another embodiment of this invention, there are 1 to 5 R²¹ groups present in formula (I), and one of the R²¹ groups is —Si(CH₃)₃.

In another embodiment of this invention, there are 2 to 5 R²¹ groups present in formula (I), and two of the R²¹ groups are the same or different —Si(R¹⁵)₃, wherein each R¹⁵ is independently selected.

In another embodiment of this invention, there are 2 to 5 R²¹ groups present in formula (I), and two of the R²¹ groups are the same or different —Si(R¹⁵)₃ and each R¹⁵ is the same or different alkyl group.

In another embodiment of this invention, there are 2 to 5 R²¹ groups present in formula (I), and two of the R²¹ groups are —Si(CH₃)₃.

In one embodiment of this invention the optional bond to X is present, the optional bond to B is absent, and X is selected from the group consisting of: —N═, —C(R⁶)═, and —C(R⁷)═.

In another embodiment of this invention the optional bond to X is absent, the optional bond to B is present, and X is selected from the group consisting of —N(R¹⁴)— and —C(R⁶)(R⁷)—.

In one embodiment, the present invention discloses compounds which are represented by structural Formula (I), or a pharmaceutically acceptable salt, solvate, ester or prodrug thereof, wherein the various moieties are described above.

In another embodiment of Formula (I),

when B is

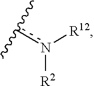

each dashed line of

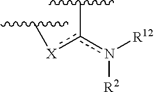

represents an optional bond with the proviso that only one optional bond (- - -) is present, and when the optional bond between the nitrogen of N(R²)(R¹²) and the adjacent ring carbon is present, then R¹² is absent (i.e. B is ═N—R²).

In another embodiment B is H.

In another embodiment B is alkoxy (e.g., methoxy, ethoxy, propoxy, butoxy, pentoxy, and hexoxy).

In another embodiment B is alkyl (e.g., methyl, ethyl, propyl, butyl, pentyl, and hexyl).

In another embodiment B is cycloalkyl (e.g., cyclopropyl, cyclobutyl, cyclopentyl, cyclohexyl, and cycloheptyl).

In another embodiment B is heterocycloalkyl (e.g., piperidinyl and pyrrolidinyl). In one example of this embodiment B is the piperidinyl moiety:

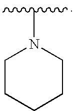

In another example of this embodiment B is the pyrrolidinyl moiety:

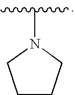

In another embodiment B is alkoxyalkyl- (e.g., CH₃—O—CH₂—, CH₃—O—CH₂—CH₂-, CH₃—O—CH₂—CH₂—CH₂—, and CH₃—O—CH₂—CH₂—CH₂—CH₂—).

In another embodiment B is hydroxylalkyl (e.g., HO—CH₂—, HO—CH₂—CH₂-, HO—CH₂—CH₂—CH₂—, and HO—CH₂—CH₂—CH₂—CH₂-).

In another embodiment B is —OR^{15a}.

In another embodiment B is ═O.

In another embodiment B is ═S.

In another embodiment B is ═N—O-alkyl (e.g., ═N—O—CH₃).

In another embodiment B is ═N—R² wherein R² is alkyl substituted with —OR¹⁵, wherein R¹⁵ is H (i.e., B is ═N-alkyl-OH, such as, for example. HO—CH₂—N═, HO—CH₂—CH₂—N═, HO—CH₂—CH₂—CH₂—N═, and HO—CH₂—CH₂—CH₂—CH₂—N═).

In another embodiment B is ═N—R² (e.g., ═NH, methoxy-N═, ethoxy-N═, propoxy-N═, butoxy-N═, pentoxy-N═, hexoxy-N═, methyl-N═, ethyl-N═, propyl-N═, butyl-N═, pentyl-N═, hexyl-N═, cyclopropyl-N═, cyclobutyl-N═, cyclopentyl-N═, cyclohexyl-N═, cycloheptyl-N═, ═O, CH₃—O—CH₂—N═, CH₃—O—CH₂—CH₂—N═, CH₃—O—CH₂—CH₂—CH₂—N═, and CH₃—O—CH₂—CH₂—CH₂—CH₂—N═).

In another embodiment, B is selected from the group consisting of ═NH, alkoxy-N═, alkyl-N═, cycloalkyl-N═, ═O, alkoxyalkyl-N═, ═S and hydroxyalkyl-N═.

In another embodiment, B is selected from the group consisting of ═NH, methoxy-N═, ethoxy-N═, propoxy-N═, butoxy-N═, pentoxy-N═, hexoxy-N═, methyl-N═, ethyl-N═, propyl-N═, butyl-N═, pentyl-N═, hexyl-N═, cyclopropyl-N═, cyclobutyl-N═, cyclopentyl-N═, cyclohexyl-N═, cycloheptyl-N═, HO—CH₂—N═, HO—CH₂—CH₂—N═, HO—CH₂—CH₂—CH₂—N═, HO—CH₂—CH₂—CH₂—CH₂—N═, ═O, CH₃—O—CH₂—N═, CH₃—O—CH₂—CH₂—N═, CH₃—O—CH₂—CH₂—CH₂—N═, and CH₃—O—CH₂—CH₂—CH₂—CH₂—N═.

In another embodiment, B is selected from the group consisting of H, alkoxy, alkyl, cycloalkyl, ═O, alkoxyalkyl-, ═S and hydroxyalkyl-.

In another embodiment, B is selected from the group consisting of H, methoxy, ethoxy, propoxy, butoxy, pentoxy, hexoxy, methyl, ethyl, propyl, butyl, pentyl, hexyl, cyclopropyl, cyclobutyl, cyclopentyl, cyclohexyl, cycloheptyl, HO—CH₂—, HO—CH₂—CH₂—, HO—CH₂—CH₂—CH₂—, HO—CH₂—CH₂—CH₂—CH₂—, ═O, CH₃—O—CH₂—, CH₃—O—CH₂—CH₂—, CH₃—O—CH₂—CH₂—CH₂—, and CH₃—O—CH₂—CH₂—CH₂—CH₂—.

In another embodiment X is —N(R¹⁴)— (e.g., X is —NH—).

In another embodiment X is —N═.

In another embodiment X is —C(R⁶)(R⁷)— (e.g., X is —CH₂).

In another embodiment X is —C(R⁶)═ or —C(R⁷)═ (e.g., X is —CH═).

In another embodiment X is —NH— and B is ═N—R².

In another embodiment X is —NH—, B is ═N—R², and W is —C(O)—.

In another embodiment X is —NH—, B is ═N—R², and W is —S(O)₂—.

In another embodiment X is —NH—, B is ═N—R², and R² is alkyl.

In another embodiment X is —NH—, B is ═N—R², R² is alkyl, and W is —C(O)—.

In another embodiment X is —NH—, B is ═N—R², R² is alkyl, and W is —S(O)₂—.

In another embodiment X is —NH—, B is ═N—R², and R² is cycloalkyl.

In another embodiment X is —NH—, B is ═N—R², R² is cycloalkyl, and W is —C(O)—.

In another embodiment X is —NH—, B is ═N—R², R² is cycloalkylalkyl, and W is —S(O)₂—.

In another embodiment X is —NH— and B is ═N-alkyl-OH (i.e., B is ═N—R² wherein R² is alkyl substituted with —OR¹⁵, and wherein R¹⁵ is H).

In another embodiment X is —NH—, B is ═N-alkyl-OH, and W is —C(O)—.

In another embodiment X is —NH—, B is ═N-alkyl-OH, and W is —S(O)₂—.

In another embodiment X is —NH—, B is ═N—R², and R² is alkoxyalkyl-.

In another embodiment X is —NH—, B is ═N—R², R² is alkoxyalkyl-, and W is —C(O)—.

In another embodiment X is —NH—, B is ═N—R², R² is alkoxyalkyl-, and W is —S(O)₂—.

In another embodiment X is —N═ and B is alkoxy.

In another embodiment X is —N═, B is alkoxy, and W is —C(O)—.

In another embodiment X is —NH—, B is alkoxy, and W is —S(O)₂—.

In another embodiment X is —N═ and B is heterocycloalkyl.

In another embodiment X is —N═, B is heterocycloalkyl, and W is —C(O)—.

In another embodiment X is —N═, B is heterocycloalkyl, and W is —S(O)₂—.

In another embodiment X is —NH— and B is ═N—O-alkyl.

In another embodiment X is —NH—, B is ═N—O-alkyl, and W is —C(O)—.

In another embodiment X is —NH—, B is ═N—O-alkyl, and W is —S(O)₂—.

In another embodiment X is —NH—, B is ═N—R², and R² is H.

In another embodiment X is —NH—, B is ═N—R², R² is H, and W is —C(O)—.

In another embodiment X is —NH—, B is ═N—R², R² is H, and W is —S(O)₂—.

In another embodiment of this invention R¹ is substituted with R²¹ groups, and at least one (e.g. 1 to 2) of the R²¹ groups is selected from the group consisting of: —SF₅, —OSF₅ and —Si(R¹⁵)₃, wherein each R¹⁵ is independently selected.

In another embodiment of this invention R¹ is substituted with R²¹ groups, and at least one (e.g. 1 to 2) of the R²¹ groups is selected from the group consisting of: —SF₅, —OSF₅ and —Si(R¹⁵)₃, and each R¹⁵ is the same or different alkyl group.

In another embodiment of this invention R¹ is substituted with R²¹ groups, and at least one (e.g. 1 to 2) of the R²¹ groups is selected from the group consisting of: —SF₅, —OSF₅ and —Si(CH₃)₃.

In another embodiment of this invention R¹ is substituted with R²¹ groups, and one R²¹ group is selected from the group consisting of: —SF₅, —OSF₅ and —Si(R¹⁵)₃, wherein each R¹⁵ is independently selected.

In another embodiment of this invention R¹ is substituted with R²¹ groups, and one R²¹ group is selected from the group consisting of: —SF₅, —OSF₅ and —Si(R¹⁵)₃, and each R¹⁵ is the same or different alkyl group.

In another embodiment of this invention R¹ is substituted with R²¹ groups, and one R²¹ group is selected from the group consisting of: —SF₅, —OSF₅ and —Si(CH₃)₃.

In another embodiment of this invention R¹ is substituted with R²¹ groups, and two R²¹ groups are selected from the group consisting of: —SF₅, —OSF₅ and —Si(R¹⁵)₃, wherein each R¹⁵ is independently selected.

In another embodiment of this invention R¹ is substituted with R²¹ groups, and two R²¹ groups are selected from the group consisting of: —SF₅, —OSF₅ and —Si(R¹⁵)₃, and each R¹⁵ is the same or different alkyl group.

In another embodiment of this invention R¹ is substituted with R²¹ groups, and two R²¹ groups are selected from the group consisting of: —SF₅, —OSF₅ and —Si(CH₃)₃.

In another embodiment of this invention R¹ is substituted with R²¹ groups, and one R²¹ group is —SF₅.

In another embodiment of this invention R¹ is substituted with R²¹ groups, and two R²¹ groups are —SF₅.

In another embodiment of this invention R¹ is substituted with R²¹ groups, and one R²¹ group is —OSF₅.

In another embodiment of this invention R¹ is substituted with R²¹ groups, and two R²¹ groups are —OSF₅.

In another embodiment of this invention R¹ is substituted with R²¹ groups, and one R²¹ group is —Si(R¹⁵)₃, wherein each R¹⁵ is independently selected.

In another embodiment of this invention R¹ is substituted with R²¹ groups, and one R²¹ group is —Si(R¹⁵)₃ and each R¹⁵ is the same or different alkyl group.

In another embodiment of this invention R¹ is substituted with R²¹ groups, and one R²¹ group is —Si(CH₃)₃.

In another embodiment of this invention R¹ is substituted with R²¹ groups, and two of the R²¹ groups are the same or different —Si(R¹⁵)₃, wherein each R¹⁵ is independently selected.

In another embodiment of this invention R¹ is substituted with R²¹ groups, and two of the R²¹ groups are the same or different —Si(R¹⁵)₃ group, and each R¹⁵ is the same or different alkyl group.

In another embodiment of this invention R¹ is substituted with R²¹ groups, and two of the R²¹ group are —Si(CH₃)₃.

In another embodiment of this invention R¹ is an alkyl group substituted with one R²¹ group, and said R²¹ group is an aryl group, and said aryl group is substituted with one or more R²² groups, and at least one (e.g., 1 to 2) R²² group is selected from the group consisting of: —SF₅, —OSF₅ and —Si(R¹⁵)₃, wherein each R¹⁵ is independently selected.

In another embodiment of this invention R¹ is an alkyl group substituted with one R²¹ group, and said R²¹ group is an aryl group, and said aryl group is substituted with one or more R²² groups, and at least one (e.g., 1 to 2) R²² group is selected from the group consisting of: —SF₅, —OSF₅ and —Si(R¹⁵)₃, and each R¹⁵ is the same or different alkyl group.

In another embodiment of this invention R¹ is an alkyl group substituted with one R²¹ group, and said R²¹ group is an aryl group, and said aryl group is substituted with one or more R²² groups, and at least one (e.g., 1 to 2) R²² group is selected from the group consisting of: —SF₅, —OSF₅ and —Si(CH₃)₃.

In another embodiment of this invention R¹ is an alkyl group substituted with one R²¹ group, and said R²¹ group is phenyl, and said phenyl is substituted with one or more R²² groups, and at least one (e.g., 1 to 2) R²² is selected from the group consisting of: —SF₅, —OSF₅ and —Si(R¹⁵)₃, wherein each R¹⁵ is independently selected.

In another embodiment of this invention R¹ is an alkyl group substituted with one R²¹ group, and said R²¹ group is phenyl, and said phenyl is substituted with one or more R²² groups, and at least one (e.g., 1 to 2) R²² is selected from the group consisting of: —SF₅, —OSF₅ and —Si(R¹⁵)₃, and each R¹⁵ is the same or different alkyl group.

In another embodiment of this invention R¹ is an alkyl group substituted with one R²¹ group, and said R²¹ group is phenyl, and said phenyl is substituted with one or more R²² groups, and at least one (e.g., 1 to 2) R²² is selected from the group consisting of: —SF₅, —OSF₅ and —Si(CH₃)₃.

In another embodiment of this invention R¹ is an alkyl group substituted with one R²¹ group, and said R²¹ group is phenyl, and said phenyl is substituted with one or more R²² groups, and one of the R²² groups is selected from the group consisting of: —SF₅, —OSF₅ and —Si(R¹⁵)₃.

In another embodiment of this invention R¹ is an alkyl group substituted with one R²¹ group, and said R²¹ group is phenyl, and said phenyl is substituted with one or more R²² groups, and one of the R²² groups is selected from the group consisting of: —SF₅, —OSF₅ and —Si(R¹⁵)₃, and each R¹⁵ is the same or different alkyl group.

In another embodiment of this invention R¹ is an alkyl group substituted with one R²¹ group, and said R²¹ group is phenyl, and said phenyl is substituted with one or more R²² groups, and one of the R²² groups is selected from the group consisting of: —SF₅, —OSF₅ and —Si(CH₃)₃.

In another embodiment of this invention R¹ is an alkyl group substituted with one R²¹ group, and said R²¹ group is phenyl, and said phenyl is substituted with one or more R²² groups, and two of the R²² groups are selected from the group consisting of: —SF₅, —OSF₅ and —Si(R¹⁵)₃.

In another embodiment of this invention R¹ is an alkyl group substituted with one R²¹ group, and said R²¹ group is phenyl, and said phenyl is substituted with one or more R²² groups, and two of the R²² groups are selected from the group consisting of: —SF₅, —OSF₅ and —Si(R¹⁵)₃, and each R¹⁵ is the same or different alkyl group.

In another embodiment of this invention R¹ is an alkyl group substituted with one R²¹ group, and said R²¹ group is phenyl, and said phenyl is substituted with one or more R²² groups, and two of the R²² groups are selected from the group consisting of: —SF₅, —OSF₅ and —Si(CH₃)₃.

In another embodiment of this invention R¹ is an alkyl group substituted with one R²¹ group, and said R²¹ group is phenyl, and said phenyl is substituted with one or more R²² groups, and one of the R²² groups is —SF₅.

In another embodiment of this invention R¹ is an alkyl group substituted with one R²¹ group, and said R²¹ group is phenyl, and said phenyl is substituted with one or more R²² groups, and two of the R²² groups are —SF₅.

In another embodiment of this invention R¹ is an alkyl group substituted with one R²¹ group, and said R²¹ group is phenyl, and said phenyl is substituted with one or more R²² groups, and one of the R²² groups is —OSF₅.

In another embodiment of this invention R¹ is an alkyl group substituted with one R²¹ group, and said R²¹ group is phenyl, and said phenyl is substituted with one or more R²² groups, and two of the R²² groups are —OSF₅.

In another embodiment of this invention R¹ is an alkyl group substituted with one R²¹ group, and said R²¹ group is phenyl, and said phenyl is substituted with one or more R²² groups, and one of the R²² groups is —Si(R¹⁵)₃.

In another embodiment of this invention R¹ is an alkyl group substituted with one R²¹ group, and said R²¹ group is phenyl, and said phenyl is substituted with one or more R²² groups, and one of the R²² groups is —Si(R¹⁵)₃, and each R¹⁵ is the same or different alkyl group.

In another embodiment of this invention R¹ is an alkyl group substituted with one R²¹ group, and said R²¹ group is phenyl, and said phenyl is substituted with one or more R²² groups, and one of the R²² groups is —Si(CH₃)₃.

In another embodiment of this invention R¹ is an alkyl group substituted with one R²¹ group, and said R²¹ group is phenyl, and said phenyl is substituted with one or more R²² groups, and two of the R²² groups are —Si(R¹⁵)₃.

In another embodiment of this invention R¹ is an alkyl group substituted with one R²¹ group, and said R²¹ group is phenyl, and said phenyl is substituted with one or more R²² groups, and two of the R²² groups are —Si(R¹⁵)₃, and each R¹⁵ is the same or different alkyl group.

In another embodiment of this invention R¹ is an alkyl group substituted with one R²¹ group, and said R²¹ group is phenyl, and said phenyl is substituted with one or more R²² groups, and two of the R²² groups are —Si(CH₃)₃.

In another embodiment of this invention R¹ is an arylalkyl- group substituted with R²¹ groups, and at least one (e.g., 1 to 2) R²¹ group is selected from the group consisting of: —SF₅, —OSF₅ and —Si(R¹⁵)₃, wherein each R¹⁵ is independently selected.

In another embodiment of this invention R¹ is an arylalkyl- group substituted with R²¹ groups, and at least one (e.g., 1 to 2) R²¹ group is selected from the group consisting of: —SF₅, —OSF₅ and —Si(R¹⁵)₃, and each R¹⁵ is the same or different alkyl group.

In another embodiment of this invention R¹ is an arylalkyl- group substituted with R²¹ groups, and at least one (e.g., 1 to 2) R²¹ group is selected from the group consisting of: —SF₅, —OSF₅ and —Si(CH₃)₃.

In another embodiment of this invention R¹ is an arylalkyl- group substituted with R²¹ groups, and said aryl moiety is phenyl, and at least one (e.g., 1 to 2) R²¹ group is selected from the group consisting of: —SF₅, —OSF₅ and —Si(R¹⁵)₃, wherein each R¹⁵ is independently selected.

In another embodiment of this invention R¹ is an arylalkyl- group substituted with R²¹ groups, and said aryl moiety is phenyl, and at least one (e.g., 1 to 2) R²¹ group is selected from the group consisting of: —SF₅, —OSF₅ and —Si(R¹⁵)₃, and each R¹⁵ is the same or different alkyl group.

In another embodiment of this invention R¹ is an arylalkyl- group substituted with R²¹ groups, and said aryl moiety is phenyl, and at least one (e.g., 1 to 2) R²¹ group is selected from the group consisting of: —SF₅, —OSF₅ and —Si(CH₃)₃.

In another embodiment of this invention R¹ is an arylalkyl- group substituted with R²¹ groups, and said aryl moiety is phenyl, and said phenyl is substituted with at least one (e.g., 1 to 3, or 1 to 2) R²¹ group, and at least one (e.g., 1 or 2) R²¹ group on said phenyl is selected from the group consisting of: —SF₅, —OSF₅ and —Si(R¹⁵)₃, wherein each R¹⁵ is independently selected.

In another embodiment of this invention R¹ is an arylalkyl- group substituted with R²¹ groups, and said aryl moiety is phenyl, and said phenyl is substituted with at least one (e.g., 1 to 3, or 1 to 2) R²¹ group, and at least one (e.g., 1 or 2) R²¹ group on said phenyl is selected from the group consisting of: —SF₅, —OSF₅ and —Si(R¹⁵)₃, and each R¹⁵ is the same or different alkyl group.

In another embodiment of this invention R¹ is an arylalkyl- group substituted with R²¹ groups, and said aryl moiety is phenyl, and said phenyl is substituted with at least one (e.g., 1 to 3, or 1 to 2) R²¹ group, and at least one (e.g., 1 or 2) R²¹ group on said phenyl is selected from the group consisting of: —SF₅, —OSF₅ and —Si(CH₃)₃.

In another embodiment of this invention R¹ is an arylalkyl- group substituted with R²¹ groups, and said aryl moiety is phenyl, and said phenyl is substituted with at least one (e.g., 1 to 3, or 1 to 2) R²¹ group, and one R²¹ group on said phenyl is selected from the group consisting of: —SF₅, —OSF₅ and —Si(R¹⁵)₃, wherein each R¹⁵ is independently selected.

In another embodiment of this invention R¹ is an arylalkyl- group substituted with R²¹ groups, and said aryl moiety is phenyl, and said phenyl is substituted with at least one (e.g., 1 to 3, or 1 to 2) R²¹ group, and one R²¹ group on said phenyl is selected from the group consisting of: —SF₅, —OSF₅ and —Si(R¹⁵)₃, and each R¹⁵ is the same or different alkyl group.

In another embodiment of this invention R¹ is an arylalkyl- group substituted with R²¹ groups, and said aryl moiety is phenyl, and said phenyl is substituted with at least one (e.g., 1 to 3, or 1 to 2) R²¹ group, and one R²¹ group on said phenyl is selected from the group consisting of: —SF₅, —OSF₅ and —Si(CH₃)₃.

In another embodiment of this invention R¹ is an arylalkyl- group substituted with R²¹ groups, and said aryl moiety is phenyl, and said phenyl is substituted with at least two (e.g., 2 to 3, or 2, or 3) R²¹ groups, and two R²¹ groups on said phenyl is selected from the group consisting of: —SF₅, —OSF₅ and —Si(R¹⁵)₃, wherein each R¹⁵ is independently selected.

In another embodiment of this invention R¹ is an arylalkyl- group substituted with R²¹ groups, and said aryl moiety is phenyl, and said phenyl is substituted with at least two (e.g., 2 to 3, or 2, or 3) R²¹ groups, and two R²¹ groups on said phenyl is selected from the group consisting of: —SF₅, —OSF₅ and —Si(R¹⁵)₃, and each R¹⁵ is the same or different alkyl group.

In another embodiment of this invention R¹ is an arylalkyl- group substituted with R²¹ groups, and said aryl moiety is phenyl, and said phenyl is substituted with at least two (e.g., 2 to 3, or 2, or 3) R²¹ groups, and two R²¹ groups on said phenyl is selected from the group consisting of: —SF₅, —OSF₅ and —Si(CH₃)₃.

In another embodiment of this invention R¹ is an arylalkyl- group substituted with R²¹ groups, and said aryl moiety is phenyl, and said phenyl is substituted with at least one (e.g., 1 to 3, or 1 to 2) R²¹ group, and one R²¹ group on said phenyl is —SF₅.

In another embodiment of this invention R¹ is an arylalkyl- group substituted with R²¹ groups, and said aryl moiety is phenyl, and said phenyl is substituted with at least one (e.g., 1 to 3, or 1 to 2) R²¹ group, and one R²¹ group on said phenyl is —OSF₅.

In another embodiment of this invention R¹ is an arylalkyl- group substituted with R²¹ groups, and said aryl moiety is phenyl, and said phenyl is substituted with at least one (e.g., 1 to 3, or 1 to 2) R²¹ group, and one R²¹ group on said phenyl is —Si(R¹⁵)₃, wherein each R¹⁵ is independently selected.

In another embodiment of this invention R¹ is an arylalkyl- group substituted with R²¹ groups, and said aryl moiety is phenyl, and said phenyl is substituted with at least one (e.g., 1 to 3, or 1 to 2) R²¹ group, and one R²¹ group on said phenyl is —Si(R¹⁵)₃, and each R¹⁵ is the same or different alkyl group.

In another embodiment of this invention R¹ is an arylalkyl- group substituted with R²¹ groups, and said aryl moiety is phenyl, and said phenyl is substituted with at least one (e.g., 1 to 3, or 1 to 2) R²¹ group, and one R²¹ group on said phenyl is —Si(CH₃)₃.

In another embodiment of this invention R¹ is an arylalkyl- group substituted with R²¹ groups, and said aryl moiety is phenyl, and said phenyl is substituted with at least two (e.g., 2 to 3) R²¹ groups, and two of the R²¹ groups on said phenyl are —SF₅.

In another embodiment of this invention R¹ is an arylalkyl- group substituted with R²¹ groups, and said aryl moiety is phenyl, and said phenyl is substituted with at least two (e.g., 2 to 3) R²¹ groups, and two of the R²¹ groups on said phenyl are —OSF₅.

In another embodiment of this invention R¹ is an arylalkyl- group substituted with R²¹ groups, and said aryl moiety is phenyl, and said phenyl is substituted with at least two (e.g., 2 to 3) R²¹ groups, and two of the R²¹ groups on said phenyl are —Si(R¹⁵)₃, wherein each R¹⁵ is independently selected.

In another embodiment of this invention R¹ is an arylalkyl- group substituted with R²¹ groups, and said aryl moiety is phenyl, and said phenyl is substituted with at least two (e.g., 2 to 3) R²¹ groups, and two of the R²¹ groups on said phenyl are —Si(R¹⁵)₃, and each R¹⁵ is the same or different alkyl group.

In another embodiment of this invention R¹ is an arylalkyl- group substituted with R²¹ groups, and said aryl moiety is phenyl, and said phenyl is substituted with at least two (e.g., 2 to 3) R²¹ groups, and two of the R²¹ groups on said phenyl are —Si(CH₃)₃.

In another embodiment, the present invention discloses a compound, or pharmaceutically acceptable salts, solvates, esters or prodrugs of said compound, said compound having the general structure shown in Formula (I) wherein:

X is —N(R¹⁴)—;

W is —C(O)—

R⁸ is H or methyl;

R¹⁰ is aryl- and said aryl- is substituted with 1-3 substitutents, which can be the same or different, each being independently selected from the group consisting of halo, alkyl, —CN, —NH₂, —NH(alkyl), —N(alkyl)₂, hydroxy and alkoxy groups;

R⁹ is heteroaryl which is substituted with 1-3 substituents which can be the same or different, each substituent being independently selected from the group consisting of halo, alkyl, CN, NH₂, NH(alkyl), N(alkyl)₂, hydroxy and alkoxy groups; and

R¹ is independently selected from the group consisting of alkyl, alkyl-OH,

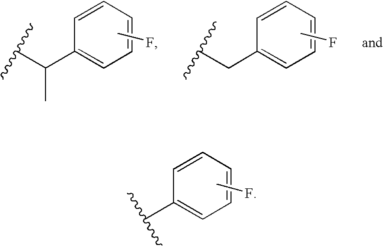

In another embodiment, R¹⁰ is

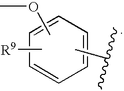

In another embodiment, R⁹ is 4-methyl-imidazol-1-yl:

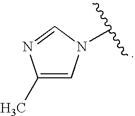

In another embodiment, R¹ is independently selected from the group consisting of alkyl, alkyl-OH, unsubstituted arylalkyl-, arylalkyl wherein said aryl- portion of arylalkyl- is substituted with 1-3 halogen, unsubstituted aryl- and aryl wherein said aryl- is substituted with 1-3 halogen.

In another embodiment, R¹⁰ is selected from the group consisting of aryl and aryl substituted with one or more R²¹ groups, and said R⁹ group is selected from the group consisting of heteroaryl and heteroaryl substituted with one or more R²¹ groups, wherein each R²¹ is independently selected.

In another embodiment of the compounds of formula (I) R¹⁰ is aryl substituted with one R²¹ group, wherein said R²¹ group is —OR¹⁵. In one example, R¹⁵ is alkyl. In another example R¹⁵ is methyl.

In another embodiment of the compounds of formula (I) R¹⁰ is phenyl substituted with one R²¹ group, wherein said R²¹ group is —OR¹⁵. In one example, R¹⁵ is alkyl. In another example R¹⁵ is methyl.

In another embodiment, R¹⁰ is phenyl substituted with one R²¹ group, and said R⁹ is imidazolyl substituted with one R²¹ group, wherein each R²¹ is independently selected.

In another embodiment of the compounds of formula (I) R¹⁰ is heteroaryl.

In another embodiment of the compounds of formula (I) R⁹ is heteroaryl.

In another embodiment of the compounds of formula (I) R⁹ is heteroaryl substituted with one or more (e.g., one) independently selected R²¹ groups.

In another embodiment of the compounds of formula (I) R⁹ is heteroaryl substituted with one or more (e.g., one) independently selected R²¹ groups, wherein each R²¹ group is the same or different alkyl group (e.g., methyl).

In another embodiment of the compounds of formula (I) R⁹ is heteroaryl substituted with one R²¹ group.

In another embodiment of the compounds of formula (I) R⁹ is heteroaryl substituted with one R²¹ group, wherein R²¹ is an alkyl group (e.g., methyl).

In another embodiment of the compounds of formula (I) R⁹ is imidazolyl.

In another embodiment of the compounds of formula (I) R⁹ is imidazolyl substituted with one or more (e.g., one) independently selected R²¹ groups.

In another embodiment of the compounds of formula (I) R⁹ is imidazolyl substituted with one or more (e.g., one) independently selected R²¹ groups, wherein each R²¹ group is the same or different alkyl group (e.g., methyl).

In another embodiment of the compounds of formula (I) R⁹ is imidazolyl substituted with one R²¹ group.

In another embodiment of the compounds of formula (I) R⁹ is imidazolyl substituted with one R²¹ group, wherein R²¹ is an alkyl group (e.g., methyl).

In another embodiment of the compounds of formula (I) R⁹ is heteroaryl, optionally substituted with one or more R²¹ groups, and R¹⁹ is aryl optionally substituted with one or more (e.g., one) R²¹ groups.

In another embodiment of the compounds of formula (I) R⁹ is heteroaryl, optionally substituted with one R²¹ group, and R¹⁹ is aryl optionally substituted with one R²¹ group.

In another embodiment of the compounds of formula (I) R⁹ is heteroaryl, optionally substituted with one or more R²¹ groups, and R¹⁹ is phenyl optionally substituted with one or more (e.g., one) R²¹ groups.

In another embodiment of the compounds of formula (I) R⁹ is heteroaryl, optionally substituted with one R²¹ group, and R¹⁰ is phenyl optionally substituted with one R²¹ group.

In another embodiment of the compounds of formula (I) R⁹ is imidazolyl, optionally substituted with one or more R²¹ groups, and R¹⁹ is aryl optionally substituted with one or more (e.g., one) R²¹ groups.

In another embodiment of the compounds of formula (I) R⁹ is imidazolyl, optionally substituted with one R²¹ group, and R¹⁹ is aryl optionally substituted with one R²¹ group.

In another embodiment of the compounds of formula (I) R⁹ is imidazolyl, optionally substituted with one or more R²¹ groups, and R¹⁹ is phenyl optionally substituted with one or more (e.g., one) R²¹ groups.

In another embodiment of the compounds of formula (I) R⁹ is imidazolyl, optionally substituted with one R²¹ group, and R¹⁹ is phenyl optionally substituted with one R²¹ group.

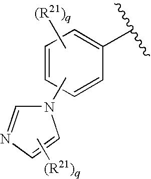

wherein q is 0, 1 or 2, such as, for example,

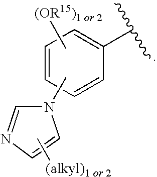

Wherein R¹⁵ is alkyl (e.g., methyl), such as, for example

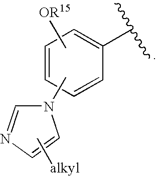

In another embodiment, the R⁹-R¹⁰-moiety is:

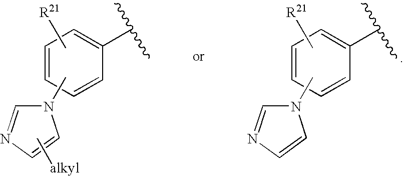

In another embodiment, the R⁹-R¹⁰-moiety is:

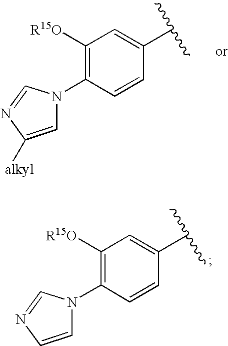

or
wherein the R⁹-R¹⁰-moiety is:

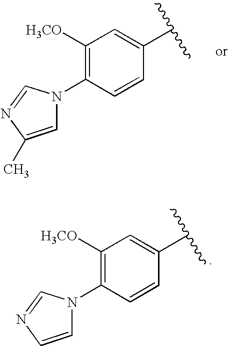

In another embodiment the R⁹R¹⁰-moiety is:

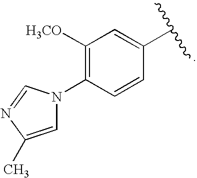

In another embodiment the R⁹-R¹⁰-moiety is:

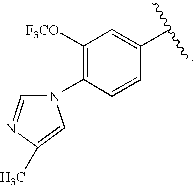

In another embodiment the R⁹-R¹⁰-moiety is:

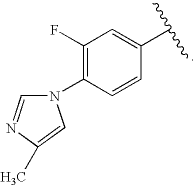

In another embodiment R⁹— R¹⁰-moiety is:

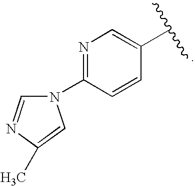

In another embodiment R⁹-R¹⁰-moiety is:

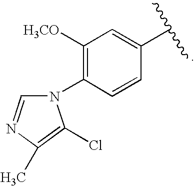

In another embodiment R⁹-R¹⁰-moiety is:

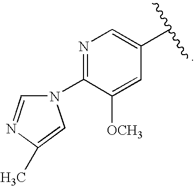

In another embodiment, R¹ group is:

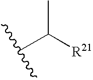

wherein R²¹ is unsubstituted or substituted with one or more independently selected R²² groups.

In another embodiment, R¹ is an alkyl group substituted with one R²¹ group, and said R²¹ group is an aryl group; or

R¹ is an alkyl group substituted with one R²¹ group, and said R²¹ group is an aryl group, and said aryl is phenyl, and said alkyl group is methyl or ethyl; or

R¹ is an alkyl group substituted with one R²¹ group, and said R²¹ group is an aryl group, and said aryl group is substituted with one or more R²² groups; or

R¹ is an alkyl group substituted with one R²¹ group, and said R²¹ group is an aryl group, and said aryl group is substituted with one or more R²² groups wherein each R²² group is the same or different halo; or

R¹ is an alkyl group substituted with one R²¹ group, and said R²¹ group is an aryl group, and said aryl group is substituted with one or two R²² halo groups; or

R¹ is an alkyl group substituted with one R²¹ group, and said R²¹ group is an aryl group, and said aryl group is substituted with one or two R²² halo groups wherein the halo is F.

In another embodiment of this invention R¹ is an alkyl group substituted with one R²¹ group, and said R²¹ group is phenyl, and said phenyl is substituted with one or more R²² groups.

In another embodiment of this invention R¹ is an alkyl group substituted with one R²¹ group, and said R²¹ group is phenyl, and said phenyl is substituted with one or more R²² groups, and each R²² group is the same or different halo.

In another embodiment of this invention R¹ is an alkyl group substituted with one R²¹ group, and said R²¹ group is phenyl, and said phenyl is substituted with one two, or three R²² halo groups, and each R²² group is the same or different halo.

In another embodiment of this invention R¹ is an alkyl group substituted with one R²¹ group, and said R²¹ group is phenyl, and said phenyl is substituted with one two, or three R²² F groups.

In another embodiment of this invention R¹ is an alkyl group substituted with one R²¹ group, and said R²¹ group is phenyl, and said phenyl is substituted with one or two R²² halo groups, and each R²² group is the same or different halo.

In another embodiment of this invention R¹ is an alkyl group substituted with one R²¹ group, and said R²¹ group is phenyl, and said phenyl is substituted with one or two R²² F groups.

In another embodiment of this invention R¹ is an ethyl group substituted with one R²¹ group, and said R²¹ group is an aryl group, and said aryl group is substituted with one or more R²² groups.

In another embodiment of this invention R¹ is a methyl group substituted with one R²¹ group, and said R²¹ group is an aryl group, and said aryl group is substituted with one or more R²² groups.

In another embodiment of this invention R¹ is an ethyl group substituted with one R²¹ group, and said R²¹ group is phenyl, and said phenyl is substituted with one or more R²² groups.

In another embodiment of this invention R¹ is a methyl group substituted with one R²¹ group, and said R²¹ group is phenyl, and said phenyl is substituted with one or more R²² groups.

In another embodiment of this invention R¹ is an ethyl group substituted with one R²¹ group, and said R²¹ group is phenyl, and said phenyl is substituted with one, two, or three R²² halo groups, and each R²² group is the same or different halo.

In another embodiment of this invention R¹ is an ethyl group substituted with one R²¹ group, and said R²¹ group is phenyl, and said phenyl is substituted with one or two R²² halo groups, and each R²² group is the same or different halo.

In another embodiment of this invention R¹ is a methyl group substituted with one R²¹ group, and said R²¹ group is phenyl, and said phenyl is substituted with one, two or three R²² halo groups, and each R²² group is the same or different halo.

In another embodiment of this invention R¹ is a methyl group substituted with one R²¹ group, and said R²¹ group is phenyl, and said phenyl is substituted with one or two R²² halo groups, and each R²² group is the same or different halo.

In another embodiment of this invention R¹ is an ethyl group substituted with one R²¹ group, and said R²¹ group is phenyl, and said phenyl is substituted with one, two, or three R²² F groups.

In another embodiment of this invention R¹ is an ethyl group substituted with one R²¹ group, and said R²¹ group is phenyl, and said phenyl is substituted with one or two R²² F groups.

In another embodiment of this invention R¹ is a methyl group substituted with one R²¹ group, and said R²¹ group is phenyl, and said phenyl is substituted with one, two or three R²² F groups.

In another embodiment of this invention R¹ is a methyl group substituted with one R²¹ group, and said R²¹ group is phenyl, and said phenyl is substituted with one or two R²² F groups.

In another embodiment of this invention R¹ is an ethyl group substituted with one R²¹ group, and said R²¹ group is phenyl, and said phenyl is substituted with one R²² halo group.

In another embodiment of this invention R¹ is a methyl group substituted with one R²¹ group, and said R²¹ group is phenyl, and said phenyl is substituted with one R²² halo group.

In another embodiment of this invention R¹ is an ethyl group substituted with one R²¹ group, and said R²¹ group is phenyl, and said phenyl is substituted with one R²² F group.

In another embodiment of this invention R¹ is a methyl group substituted with one R²¹ group, and said R²¹ group is phenyl, and said phenyl is substituted with one R²² F group.

In another embodiment of this invention R¹ is an ethyl group substituted with one R²¹ group, and said R²¹ group is phenyl, and said phenyl is substituted with two of the same or different R²² halo groups.

In another embodiment of this invention R¹ is a methyl group substituted with one R²¹ group, and said R²¹ group is phenyl, and said phenyl is substituted with two of the same or different R²² halo groups.

In another embodiment of this invention R¹ is an ethyl group substituted with one R²¹ group, and said R²¹ group is phenyl, and said phenyl is substituted with two R²² F groups.

In another embodiment of this invention R¹ is a methyl group substituted with one R²¹ group, and said R²¹ group is phenyl, and said phenyl is substituted with two R²² F group.

In another embodiment of this invention R¹ is an ethyl group substituted with one R²¹ group, and said R²¹ group is phenyl, and said phenyl is substituted with three of the same or different R²² halo groups.

In another embodiment of this invention R¹ is a methyl group substituted with one R²¹ group, and said R²¹ group is phenyl, and said phenyl is substituted with three of the same or different R²² halo groups.

In another embodiment of this invention R¹ is an ethyl group substituted with one R²¹ group, and said R²¹ group is phenyl, and said phenyl is substituted with three R²² F groups.

In another embodiment of this invention R¹ is a methyl group substituted with one R²¹ group, and said R²¹ group is phenyl, and said phenyl is substituted with three R²² F group.

In another embodiment of this invention R¹ is an alkyl group substituted with one or more independently selected R²¹ groups.

In another embodiment of this invention R¹ is:

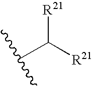

wherein each R²¹ is independently selected, and each R²¹ is independently unsubstituted or substituted with one or more independently selected R²² groups.

In another embodiment of this invention R¹ is:

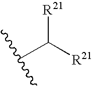

wherein one R²¹ is an unsubstituted or substituted alkyl group.

In another embodiment of this invention R¹ is:

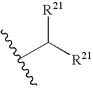

wherein one R²¹ is an unsubstituted alkyl group.

In another embodiment of this invention R¹ is:

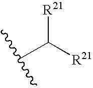

wherein one R²¹ is a substituted alkyl group.

In another embodiment of this invention R¹ is:

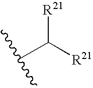

wherein one R²¹ is an unsubstituted or substituted alkyl group, and the other R²¹ is an unsubstituted or substituted aryl (e.g., phenyl) group.

In another embodiment of this invention R¹ is:

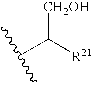

and R²¹ is unsubstituted or substituted with one or more independently selected R²² groups.

In another embodiment of this invention R¹ is:

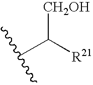

and R²¹ is unsubstituted aryl (e.g., phenyl) or aryl (e.g., phenyl) substituted with one or more independently selected R²² groups.

Other embodiments of the compounds of formula (I) are directed to any one of the embodiments directed to R¹ being an alkyl substituted with one R²¹ group, wherein said alkyl is

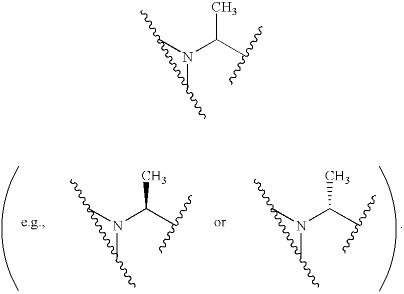

Other embodiments of the compounds of formula (I) are directed to any one of the embodiments directed to R¹ being an alkyl substituted with one R²¹ group, wherein said alkyl is

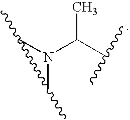

Other embodiments of the compounds of formula (I) are directed to any one of the embodiments directed to R¹ being an alkyl substituted with one R²¹ group, wherein said alkyl is

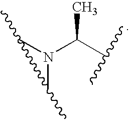

Other embodiments of the compounds of formula (I) are directed to any one of the embodiments directed to R¹ being an alkyl substituted with one R²¹ group, wherein said alkyl is

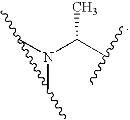

In another embodiment of the compounds of formula (I) R¹ is:

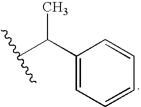

In another embodiment of the compounds of formula (I) R¹ is:

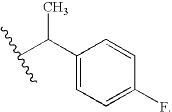

In another embodiment of the compounds of formula (I) R¹ is:

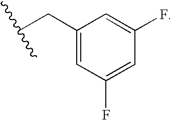

In another embodiment of the compounds of formula (I) R¹ is:

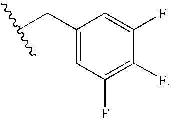

In another embodiment of the compounds of formula (I) R¹ is:

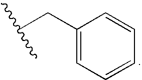

In another embodiment of the compounds of formula (I) R¹ is:

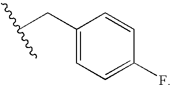

In another embodiment of the compounds of formula (I) R¹ is:

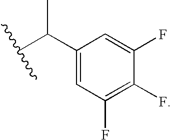

In another embodiment of the compounds of formula (I) R¹ is:

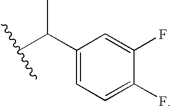

In another embodiment of the compounds of formula (I) R¹ is:

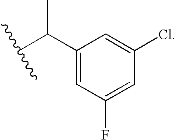

In another embodiment of the compounds of formula (I) R¹ is:

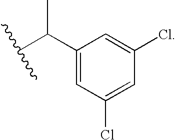

In another embodiment of the compounds of formula (I) R¹ is:

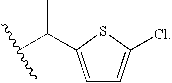

In another embodiment of the compounds of formula (I) R¹ is:

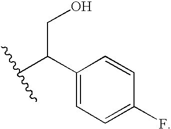

In another embodiment of the compounds of formula (I) R¹ is:

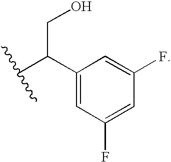

In another embodiment of the compounds of formula (I) R¹ is:

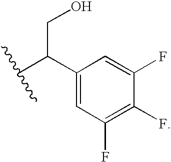

In another embodiment of the compounds of formula (I) R¹ is:

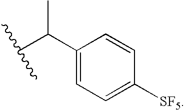

In another embodiment of the compounds of formula (I) R¹ is:

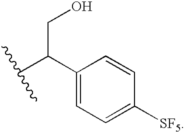

In another embodiment of the compounds of formula (I) R¹ is:

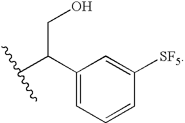

In another embodiment of the compounds of formula (I) R¹ is:

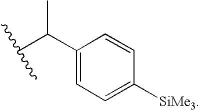

In another embodiment of the compounds of formula (I) R¹ is:

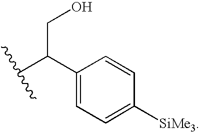

In another embodiment of the compounds of formula (I) R¹ is:

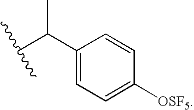

In another embodiment of the compounds of formula (I) R¹ is:

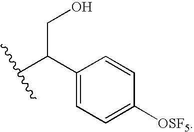

In another embodiment of the compounds of formula (I) R¹ is:

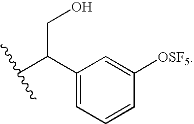

In another embodiment of this invention R¹ is selected from the group consisting of:

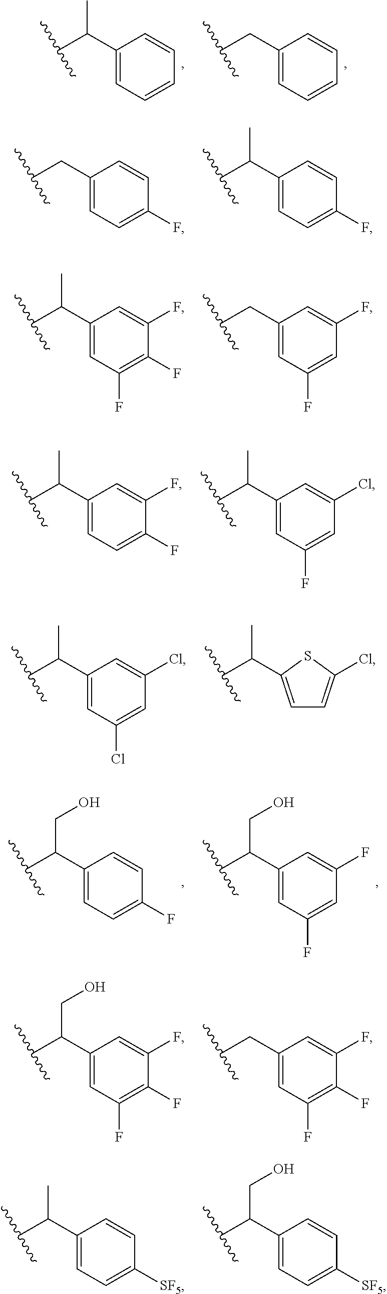

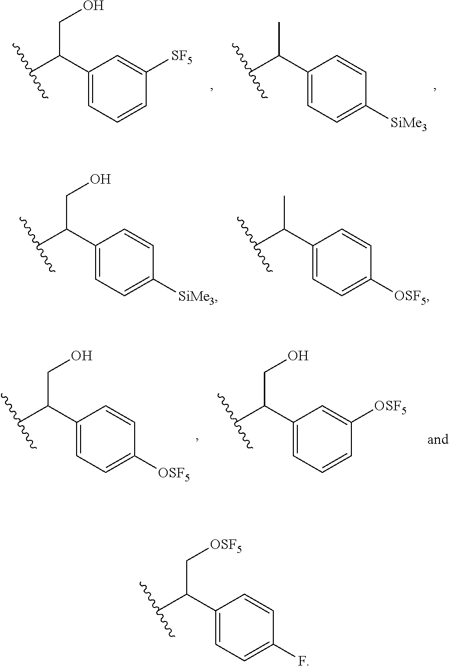

In another embodiment of this invention R¹ is selected from the group consisting of:

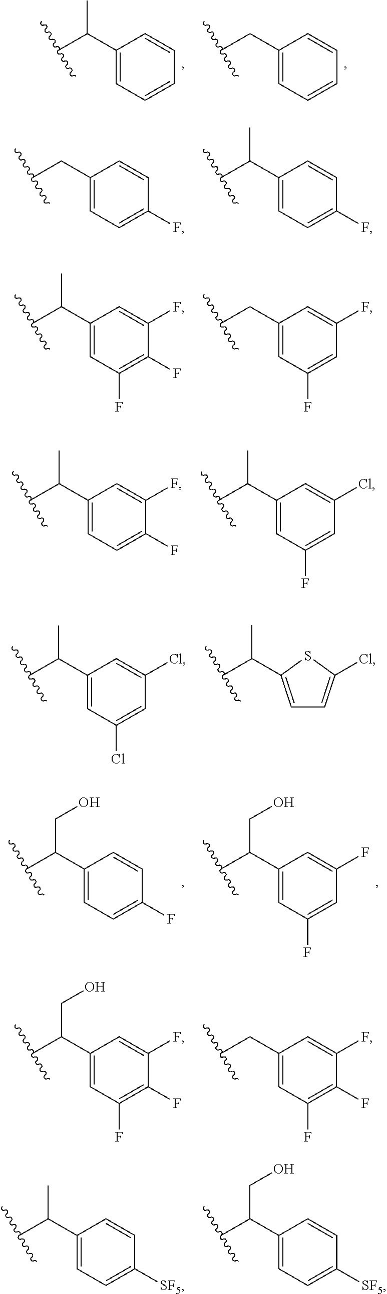

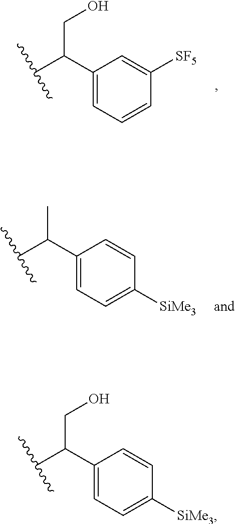

In another embodiment of this invention R¹ is selected from the group consisting of:

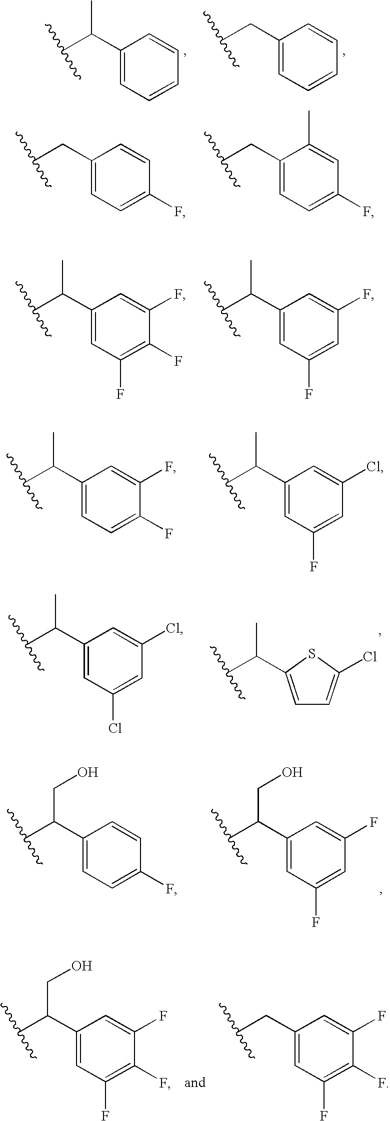

In another embodiment, R¹ is selected from the group consisting of:

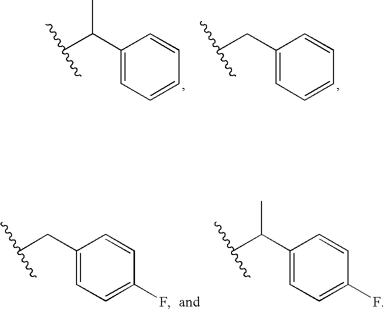

In another embodiment of this invention R¹ is selected from the group consisting of:

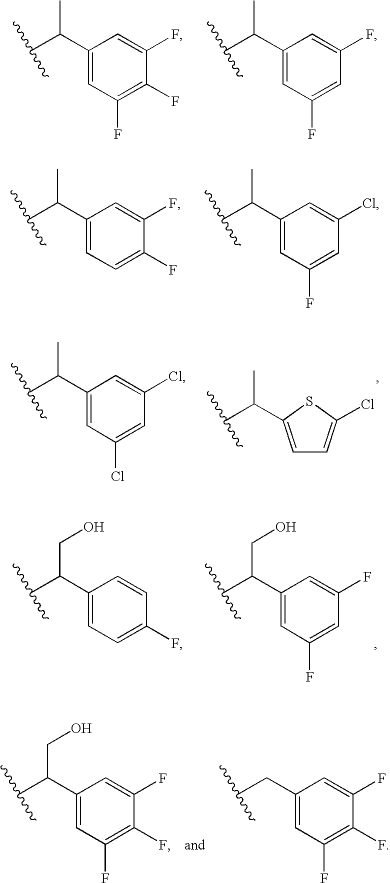

In another embodiment R¹ is selected from the group consisting of:

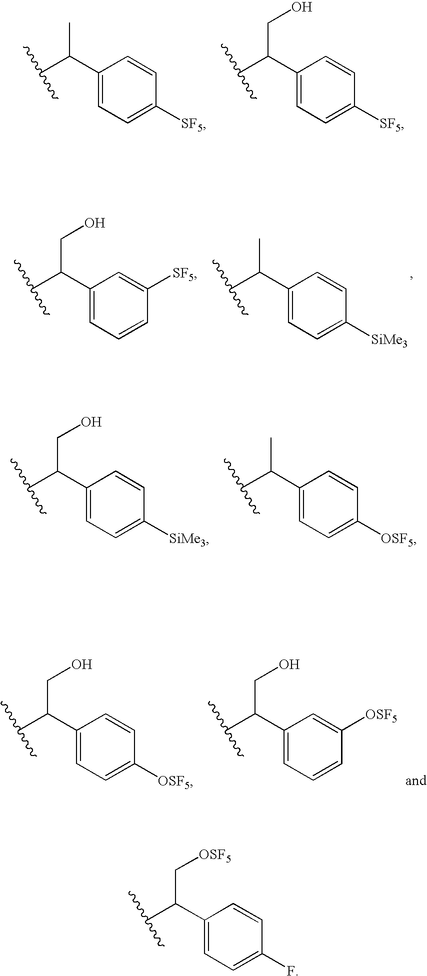

In another embodiment R¹ is selected from the group consisting of:

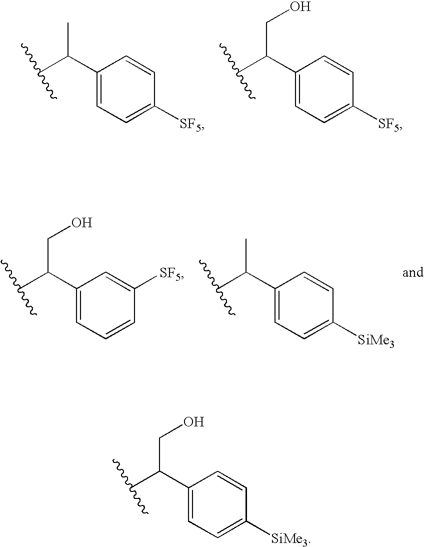

In another embodiment R¹ is selected from the group consisting of:

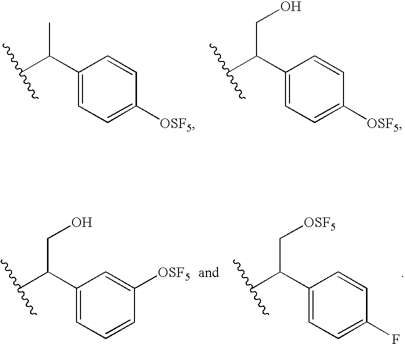

In another embodiment, R¹⁰ is selected from the group consisting of heteroaryl and heteroaryl substituted with one or more R²¹ groups, and said R⁹ group is selected from the group consisting of heteroaryl (e.g., imidazolyl) and heteroaryl (e.g., imidazolyl) substituted with one or more (e.g., one or two, or one) R²¹ groups (e.g., alkyl, such as, for example, methyl), and wherein each R²¹ is independently selected.

In another embodiment, (1)
R¹ is an alkyl group substituted with one R²¹ group, or
R¹ is an alkyl group substituted with one R²¹ group, and said R²¹ group is substituted with one or more independently selected R²² groups, and
R¹⁰ is selected from the group consisting of aryl and aryl substituted with one or more independently selected R²¹ groups, and
R⁹ is selected from the group consisting of heteroaryl and heteroaryl substituted with one or more independently selected R²¹ groups.

In another embodiment, (2)
R¹ is an alkyl group substituted with one phenyl, or
R¹ is an alkyl group substituted with one phenyl, and said phenyl is substituted with one or more independently selected R²² groups, and
R¹⁰ is selected from the group consisting of phenyl and phenyl substituted with one or more independently selected R²¹ groups, and
R⁹ is selected from the group consisting of imidazolyl and imidazolyl substituted with one or more independently selected R²¹ groups.

In another embodiment, (3)
R¹ is a methyl or ethyl group substituted with one phenyl, or
R¹ is a methyl or ethyl group substituted with one phenyl, and said phenyl is substituted with one or more independently selected halos, and
R¹⁰ is selected from the group consisting of phenyl and phenyl substituted with one or more independently selected —OR¹⁵ groups, and
R⁹ is selected from the group consisting of imidazolyl and imidazolyl substituted with one or more independently selected alkyl groups.

In another embodiment, (4)
R¹ is a methyl or ethyl group substituted with one phenyl, or
R¹ is an methyl or ethyl group substituted with one phenyl, and said phenyl is substituted with one or two independently selected halos, and
R¹⁰ is selected from the group consisting of phenyl and phenyl substituted with one or two independently selected —OR¹⁵ groups, wherein R¹⁵ is alkyl, and
R⁹ is selected from the group consisting of imidazolyl and imidazolyl substituted with one or two independently selected alkyl groups.

In another embodiment, (5)
R¹ is a methyl or ethyl group substituted with one phenyl, or
R¹ is an methyl or ethyl group substituted with one phenyl, and said phenyl is substituted with one or two F, and
R¹⁰ is selected from the group consisting of phenyl and phenyl substituted with one or two independently selected —OR¹⁵ groups, wherein R¹⁵ is methyl, and
R⁹ is selected from the group consisting of imidazolyl and imidazolyl substituted with one or two independently selected methyl groups.

In another embodiment, (6)
R¹ is a methyl or ethyl group substituted with one phenyl, or
R¹ is an methyl or ethyl group substituted with one phenyl, and said phenyl is substituted with one or two F, and
R¹⁰ is phenyl substituted with one —OR¹⁵ group, wherein R¹⁵ is methyl, and
R⁹ is selected from the group consisting of imidazolyl and imidazolyl substituted with one methyl group.

In another embodiment, (7)
R¹ is selected from the group consisting of:

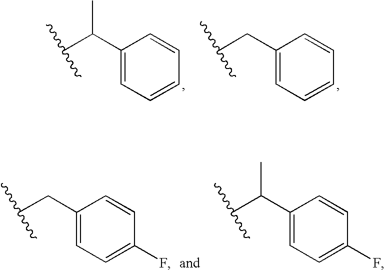

and wherein the R⁹-R¹⁰- moiety is:

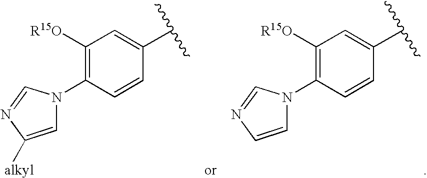

In another embodiment, (8)
R¹ is selected from the group consisting of:

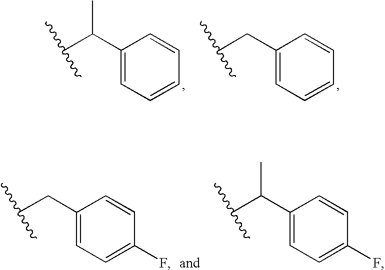

and wherein the R⁹-R¹⁰-moiety is:

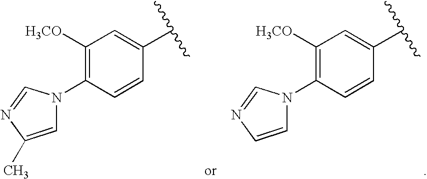

In another embodiment R¹ is selected from the group consisting of:

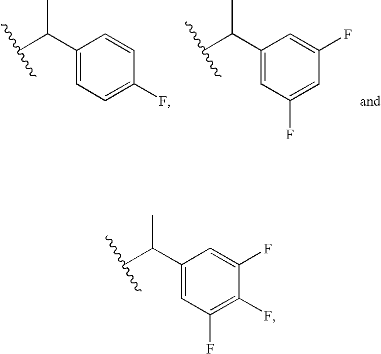

and wherein the R⁹-R¹⁰-moiety is:

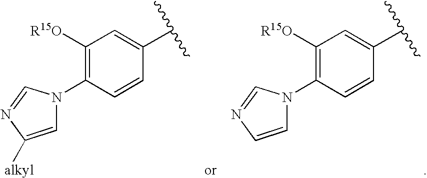

In another embodiment R¹ is selected from the group consisting of:

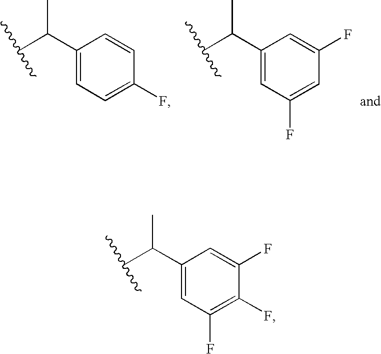

and wherein the R⁹-R¹⁰-moiety is:

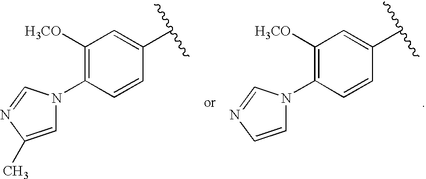

In another embodiment of this invention R¹ is selected from the group consisting of:

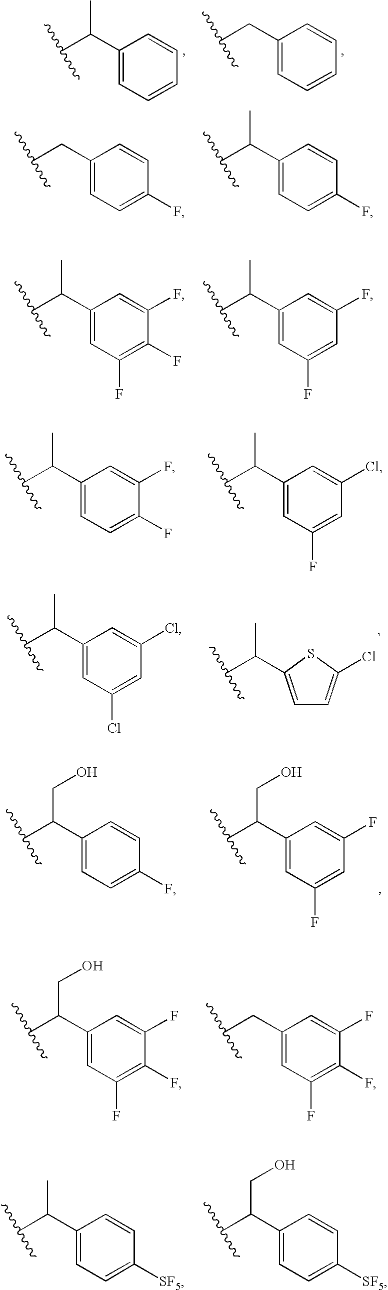

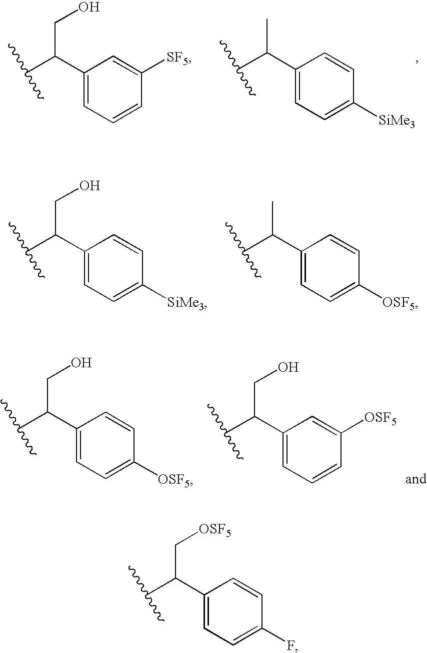

and
the R⁹-R¹⁰-moiety is:

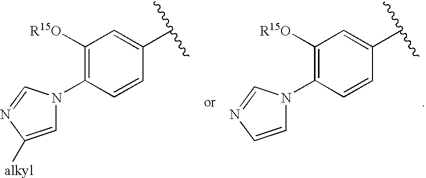

In another embodiment of this invention R¹ is selected from the group consisting of:

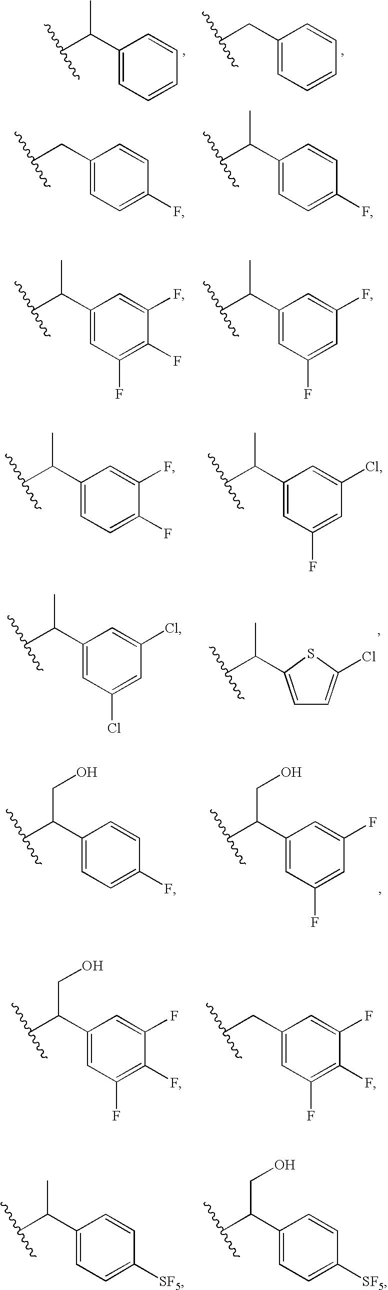

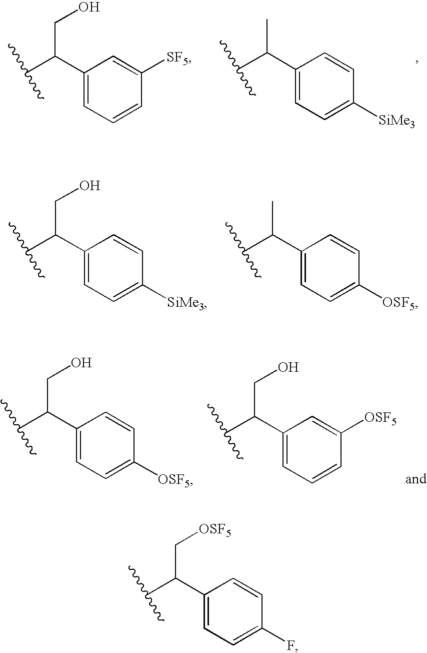

and
the R⁹-R¹⁰-moiety is:

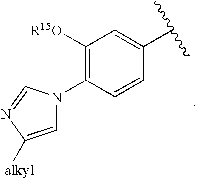

In another embodiment of this invention R¹ is selected from the group consisting of:

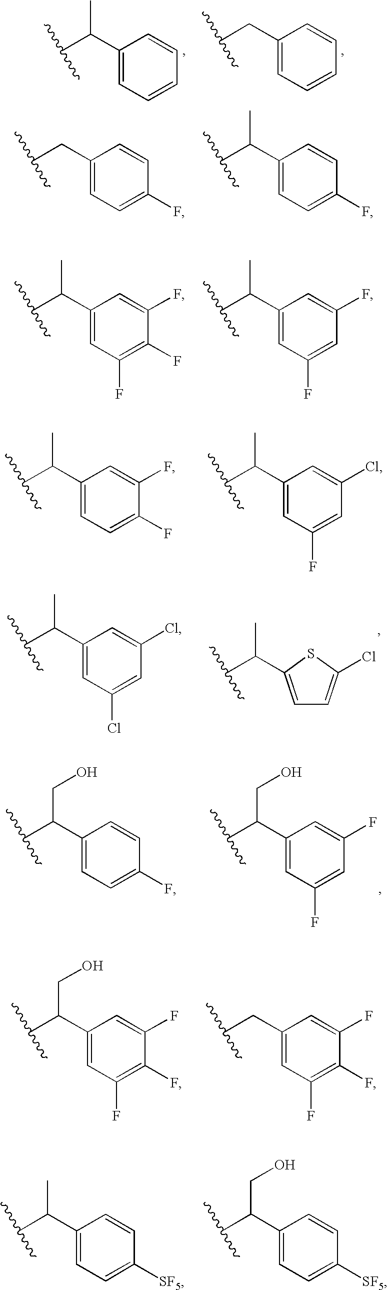

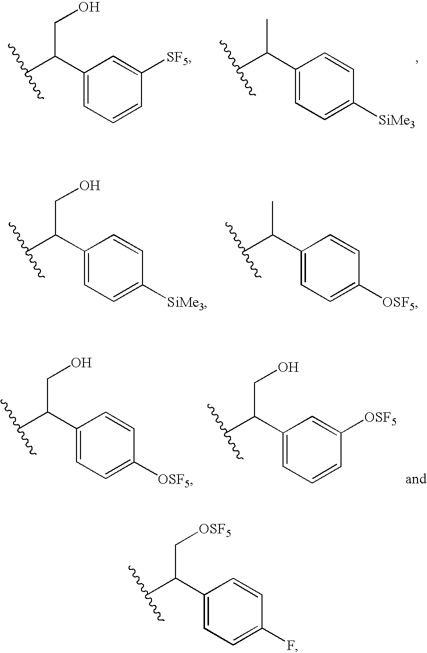

and
the R⁹-R¹⁰-moiety is:

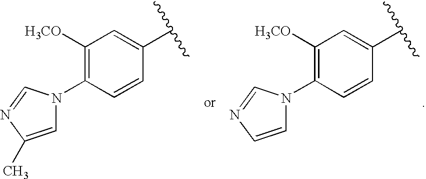

In another embodiment of this invention R¹ is selected from the group consisting of:

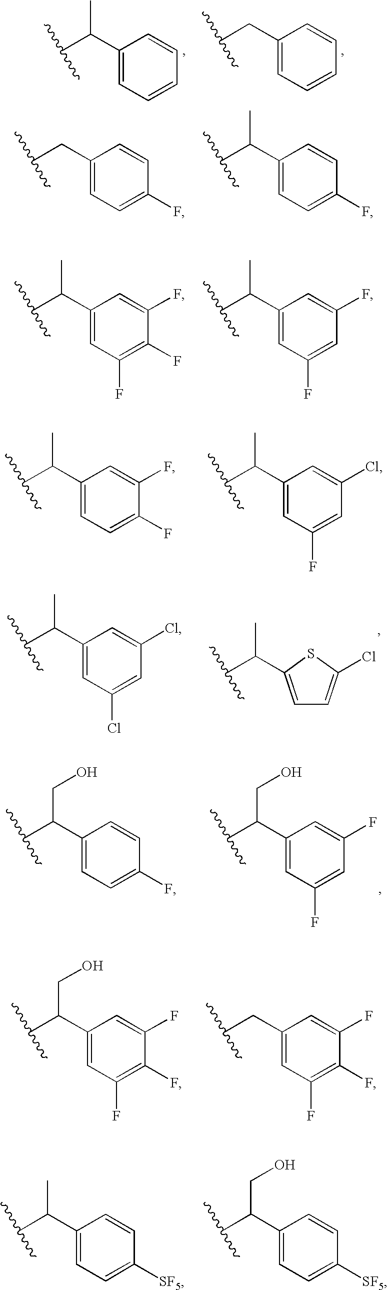

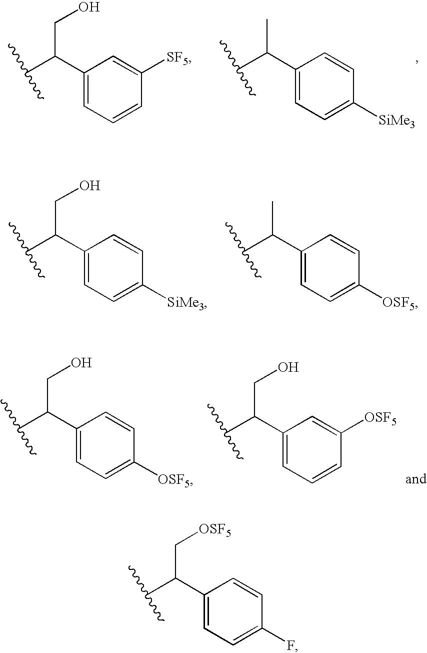

and
the R⁹-R¹⁰-moiety is:

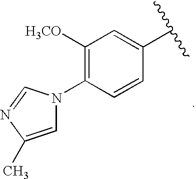

In another embodiment of this invention R¹ is selected from the group consisting of:

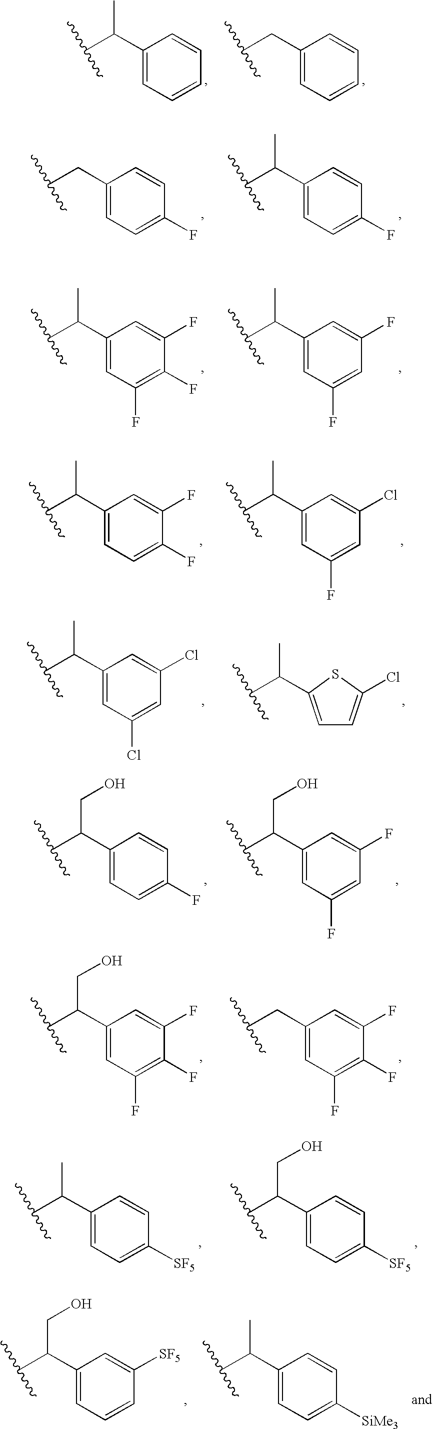

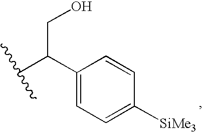

and the R⁹-R¹⁰-moiety is:

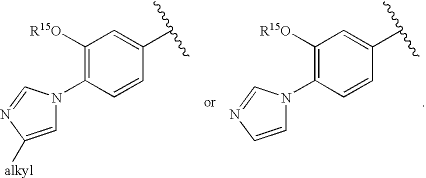

In another embodiment of this invention R¹ is selected from the group consisting of:

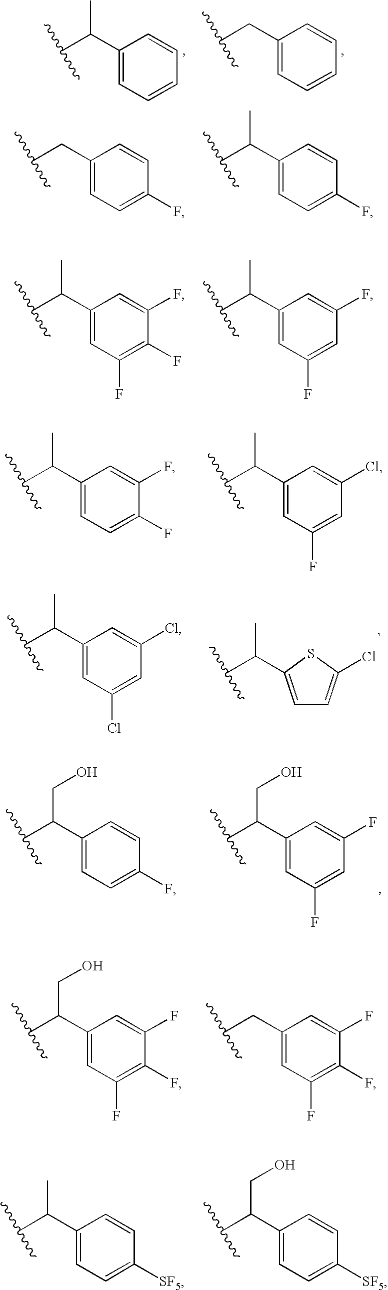

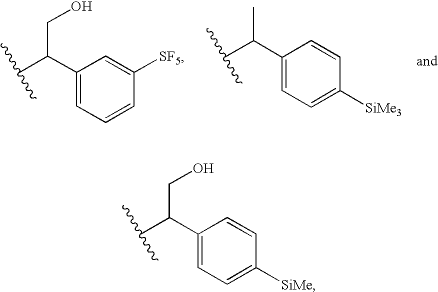

and
the R⁹-R¹⁰-moiety is:

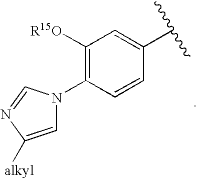

In another embodiment of this invention R¹ is selected from the group consisting of:

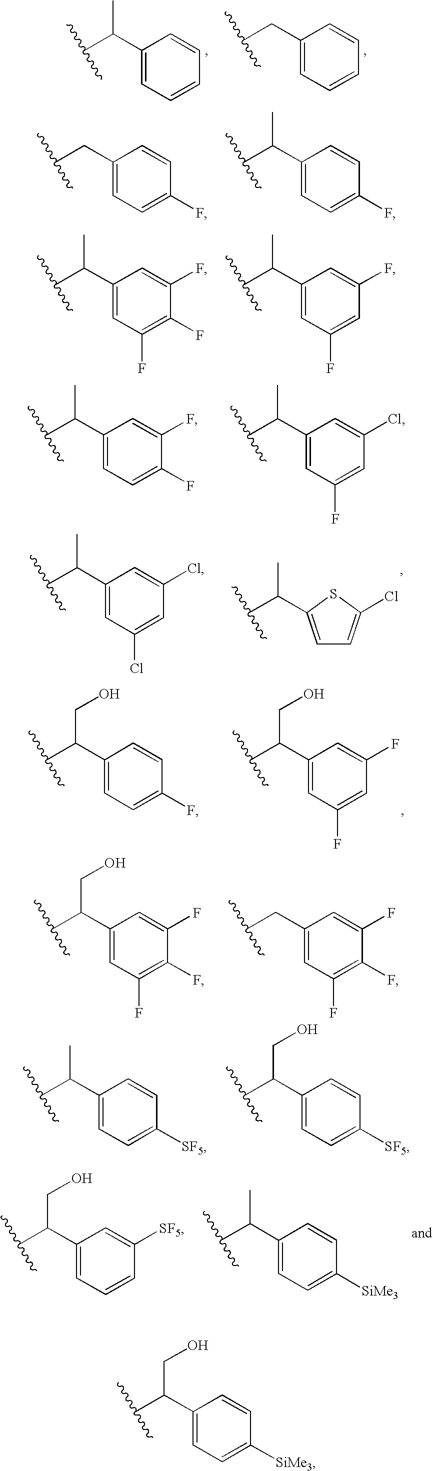

and
the R⁹-R¹⁰-moiety is:

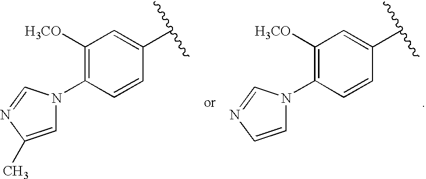

In another embodiment of this invention R¹ is selected from the group consisting of:

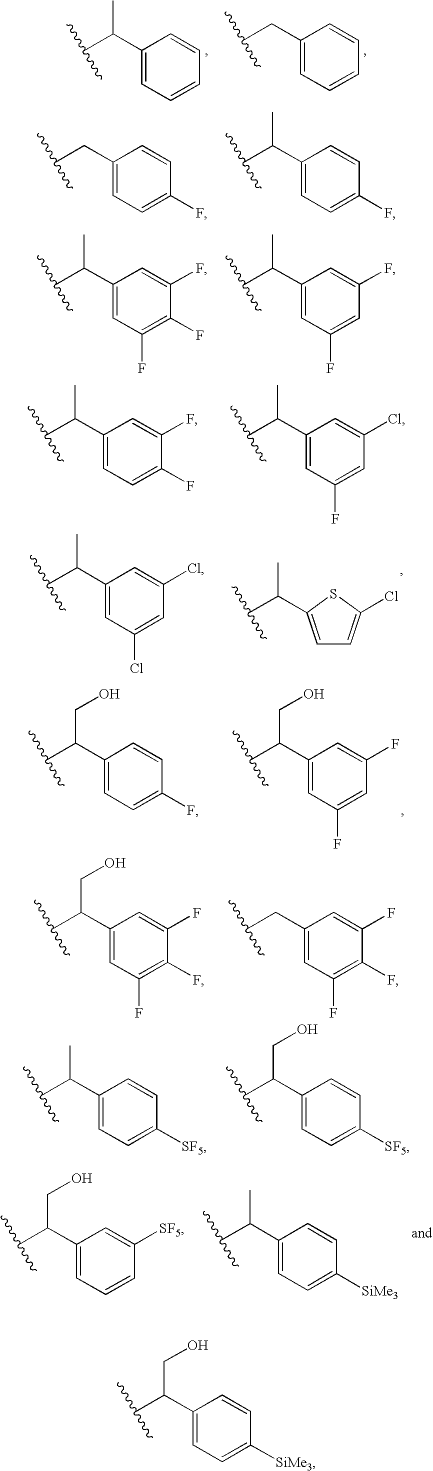

and
the R⁹-R¹⁰-moiety is:

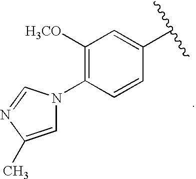

In another embodiment of this invention R¹ is selected from the group consisting of:

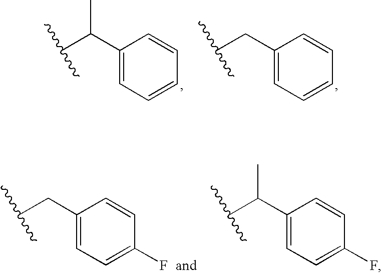

and wherein the R⁹-R¹⁰-moiety is:

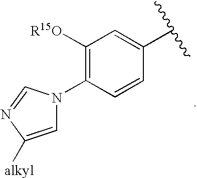

In another embodiment of this invention R¹ is selected from the group consisting of:

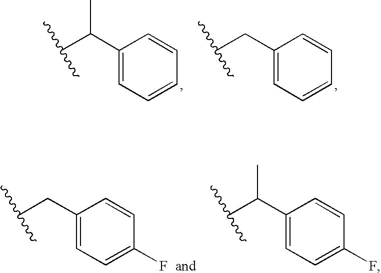

and wherein the R⁹-R¹⁰-moiety is:

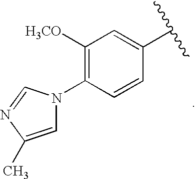

In another embodiment of this invention R¹ is selected from the group consisting of:

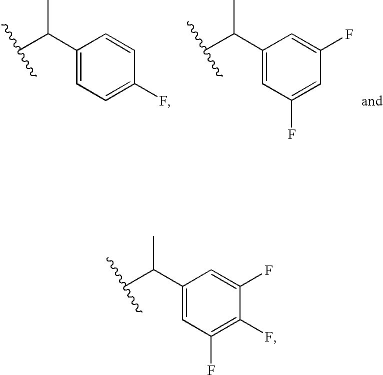

and wherein the R⁹-R¹⁰-moiety is:

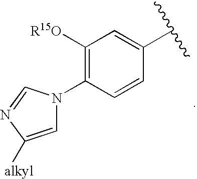

In another embodiment of this invention R¹ is selected from the group consisting of:

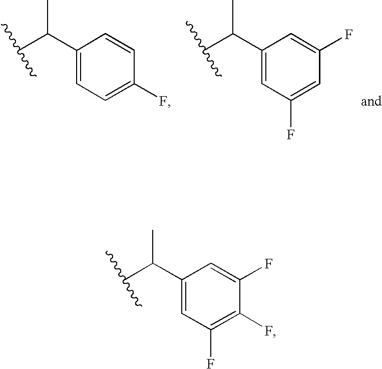

and wherein the R⁹-R¹⁰-moiety is:

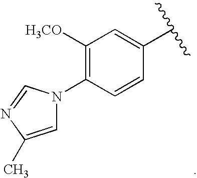

In another embodiment of this invention R¹ is selected from the group consisting of:

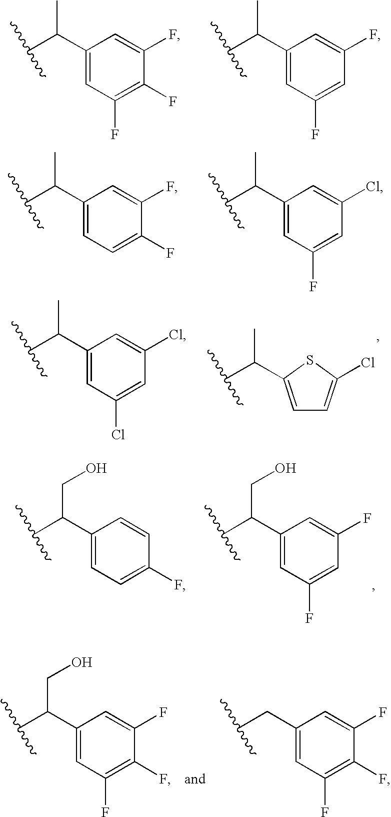

and
the R⁹-R¹⁰-moiety is:

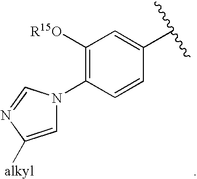

In another embodiment of this invention R¹ is selected from the group consisting of:

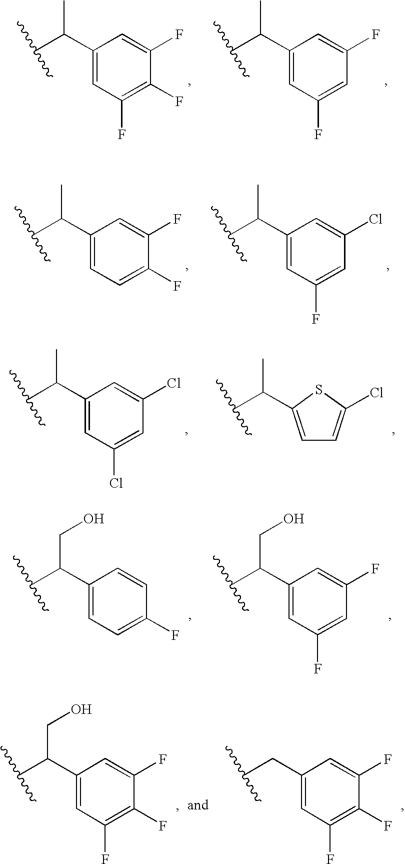

and
the R⁹-R¹⁰-moiety is:

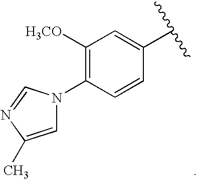

In another embodiment of this invention R¹ is selected from the group consisting of:

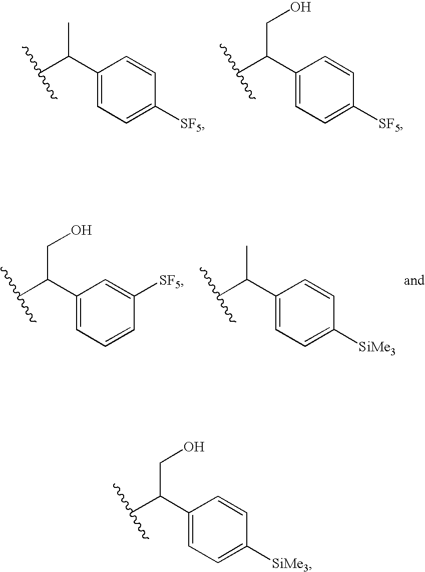

and
the R⁹-R¹⁰-moiety is:

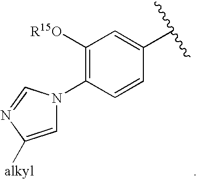

In another embodiment of this invention R¹ is selected from the group consisting of:

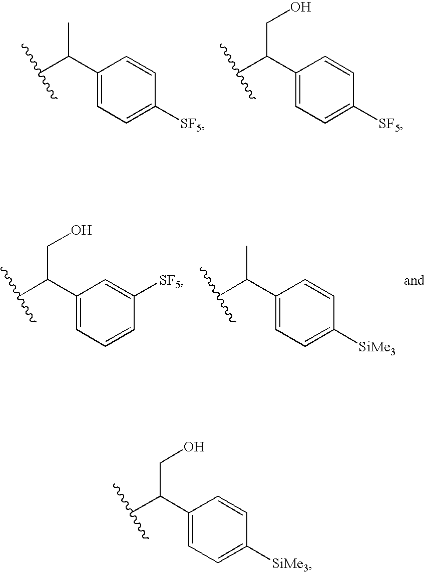

and
the R⁹-R¹⁰-moiety is:

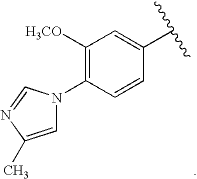

In another embodiment of this invention R¹ is selected from the group consisting of:

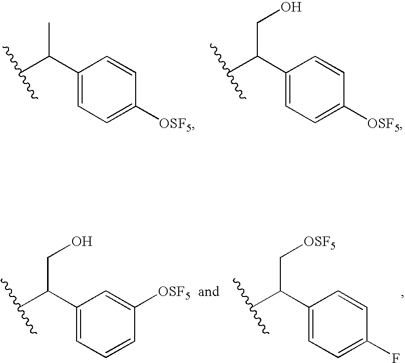

and
the R⁹-R¹⁰-moiety is:

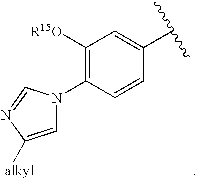

In another embodiment of this invention R¹ is selected from the group consisting of:

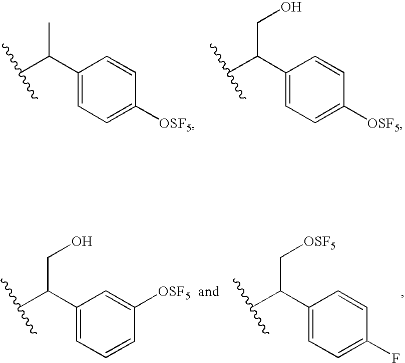

and
the R⁹-R¹⁰-moiety is:

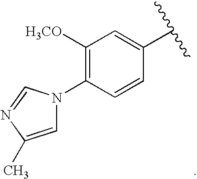

In another embodiment, W is —C(O)—.

In another embodiment W is —S(O)₂—.

In another embodiment, B is selected from the group consisting of B is

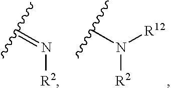

### —OR^{15a}, ═O or ═S.

In another embodiment, the present application discloses a compound, or pharmaceutically acceptable salts, solvates, esters or prodrugs of said compound, said compound having the general structures shown below:

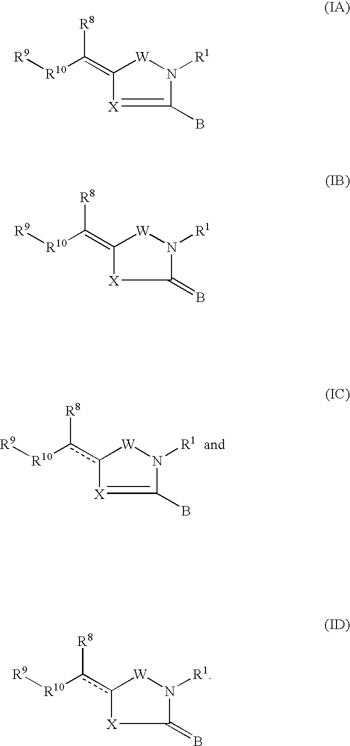

In another embodiment, the present application discloses a compound, or pharmaceutically acceptable salts, solvates, esters or prodrugs of said compound, said compound having the general structures shown below:

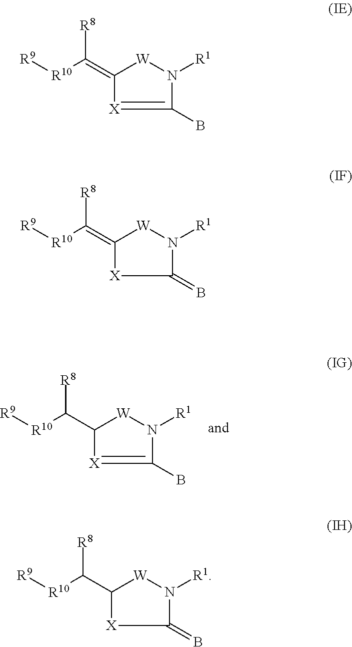

In another embodiment, the present application discloses a compound, or pharmaceutically acceptable salts, solvates, esters or prodrugs of said compound, said compound having the general structures shown below:

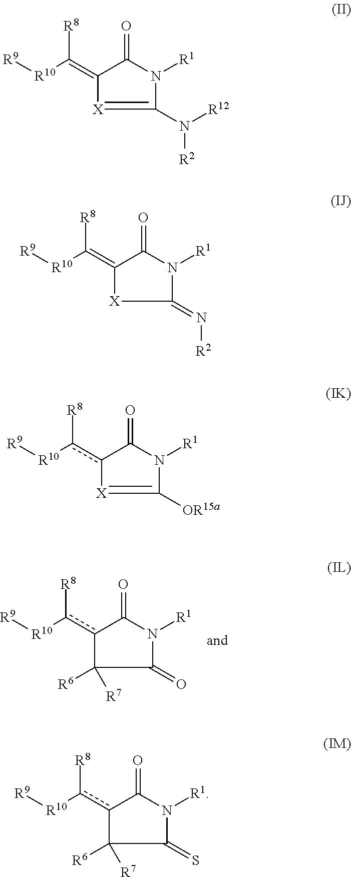

Another embodiment is directed to a compound of formula (IA), or a pharmaceutically acceptable salt, ester or solvate thereof.

Another embodiment is directed to a compound of formula (IB), or a pharmaceutically acceptable salt, ester or solvate thereof.

Another embodiment is directed to a compound of formula (IC), or a pharmaceutically acceptable salt, ester or solvate thereof.

Another embodiment is directed to a compound of formula (ID), or a pharmaceutically acceptable salt, ester or solvate thereof.

Another embodiment is directed to a compound of formula (IE), or a pharmaceutically acceptable salt, ester or solvate thereof.

Another embodiment is directed to a compound of formula (IF), or a pharmaceutically acceptable salt, ester or solvate thereof.

Another embodiment is directed to a compound of formula (IG), or a pharmaceutically acceptable salt, ester or solvate thereof.

Another embodiment is directed to a compound of formula (I)H, or a pharmaceutically acceptable salt, ester or solvate thereof.

Another embodiment is directed to a compound of formula (II), or a pharmaceutically acceptable salt, ester or solvate thereof.

Another embodiment is directed to a compound of formula (IJ), or a pharmaceutically acceptable salt, ester or solvate thereof.

Another embodiment is directed to a compound of formula (IK), or a pharmaceutically acceptable salt, ester or solvate thereof.

Another embodiment is directed to a compound of formula (IL), or a pharmaceutically acceptable salt, ester or solvate thereof.

Another embodiment is directed to a compound of formula (IM), or a pharmaceutically acceptable salt, ester or solvate thereof.

Another embodiment is directed to a compound of formula (IA).

Another embodiment is directed to a compound of formula (IB).

Another embodiment is directed to a compound of formula (IC).

Another embodiment is directed to a compound of formula (ID). Another embodiment is directed to a compound of formula (IE).

Another embodiment is directed to a compound of formula (IF).

Another embodiment is directed to a compound of formula (IG).

Another embodiment is directed to a compound of formula (IH).

Another embodiment is directed to a compound of formula (II). Another embodiment is directed to a compound of formula (IJ).

Another embodiment is directed to a compound of formula (IK).

Another embodiment is directed to a compound of formula (IL).

Another embodiment is directed to a compound of formula (IM).

In another embodiment, X is —N(R¹⁴)— and the compound of formula (I) is selected from the group consisting of: 16, ID, IF, IH, and IJ.

In another embodiment, X is —N═ and the compound of formula (I) is selected from the group consisting of: IA, IC, IE, IG, II, and IK.

In another embodiment, X is —N═ and the compound of formula (I) is (IA).

In another embodiment, X is —N(R¹⁴)— and the compound of formula (I) is (IB).

In another embodiment, X is —N═ and the compound of formula (I) is (IC. In another embodiment, X is —N(R¹⁴)— and the compound of formula (I) is (ID).

In another embodiment, X is —N═ and the compound of formula (I) is (IE).

In another embodiment, X is —N(R¹⁴)— and the compound of formula (I) is (IF).

In another embodiment, X is —N═ and the compound of formula (I) is (IG).

In another embodiment, X is —N(R¹⁴)— and the compound of formula (I) is (IH).

In another embodiment, X is —N═ and the compound of formula (I) is (II).

In another embodiment, X is —N(R¹⁴)— and the compound of formula (I) is (IJ).

In another embodiment, X is —N═ and the compound of formula (I) is (IK).

In another embodiment of this invention R¹ is selected from the group consisting of:

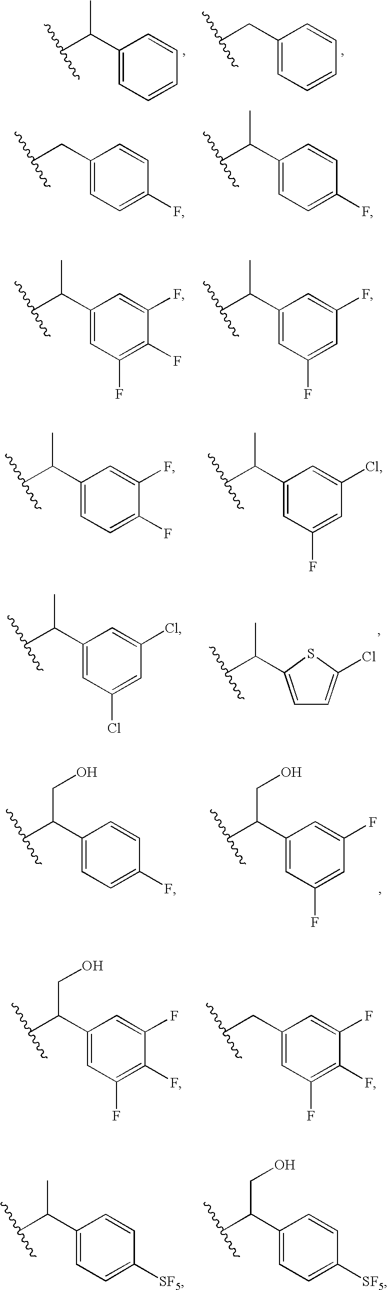

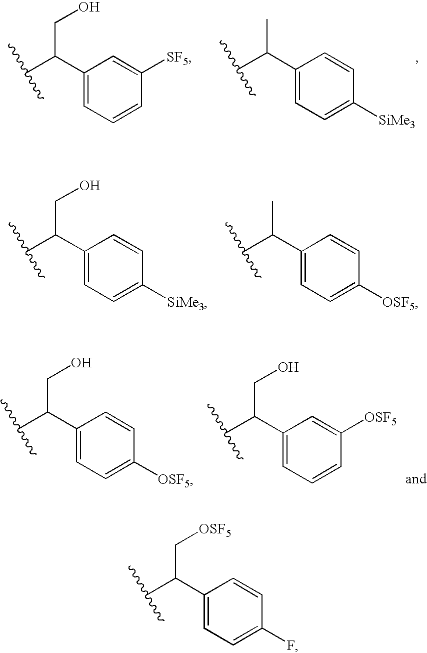

and
the R⁹-R¹⁰-moiety is:

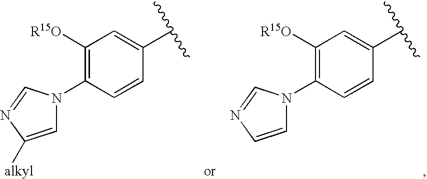

and
X is —N(R¹⁴)— and the compound of formula (I) is selected from the group consisting of: IB, ID, IF, IH, and IJ.

In another embodiment of this invention R¹ is selected from the group consisting of:

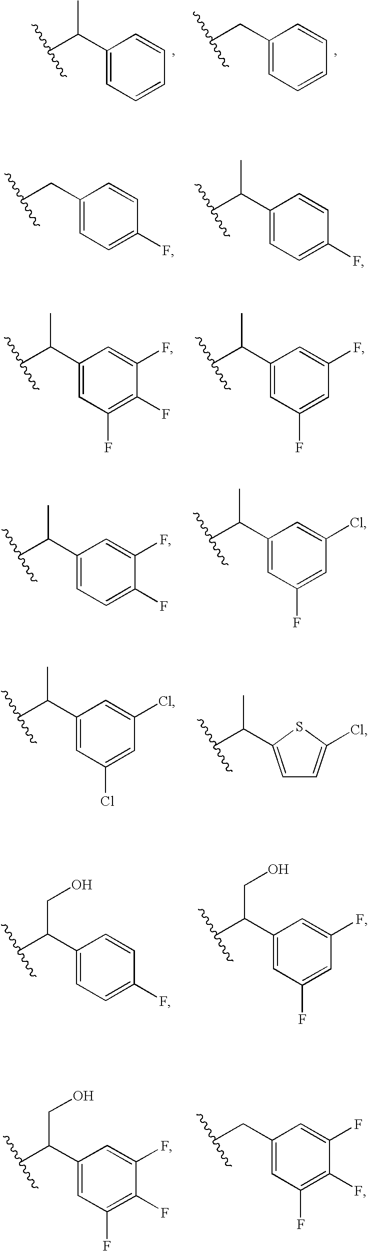

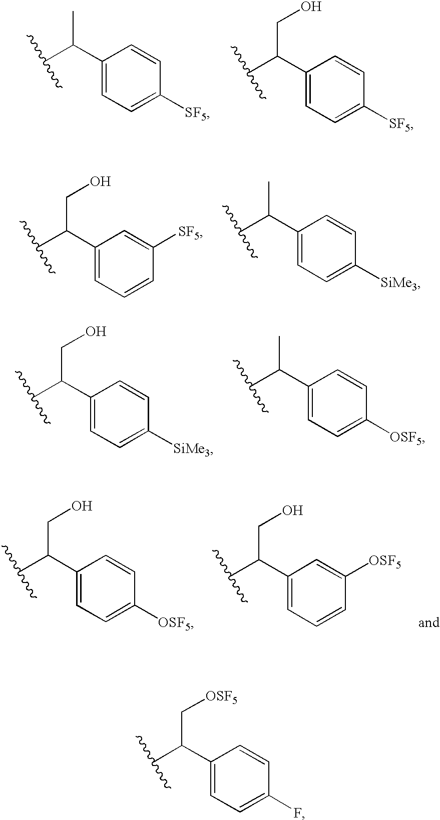

and
the R⁹-R¹⁰-moiety is:

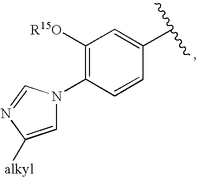

and
X is —N(R¹⁴)— and the compound of formula (I) is selected from the group consisting of: IB, ID, IF, IH, and IJ.

In another embodiment of this invention R¹ is selected from the group consisting of:

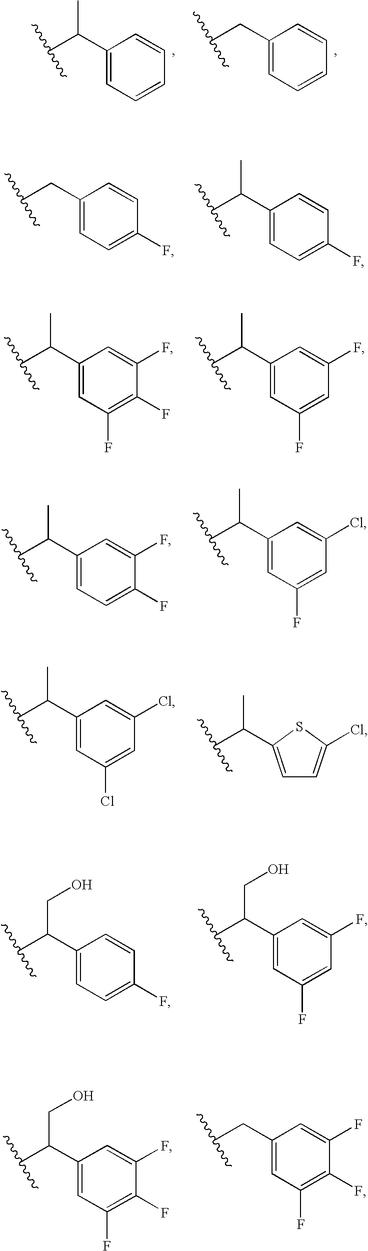

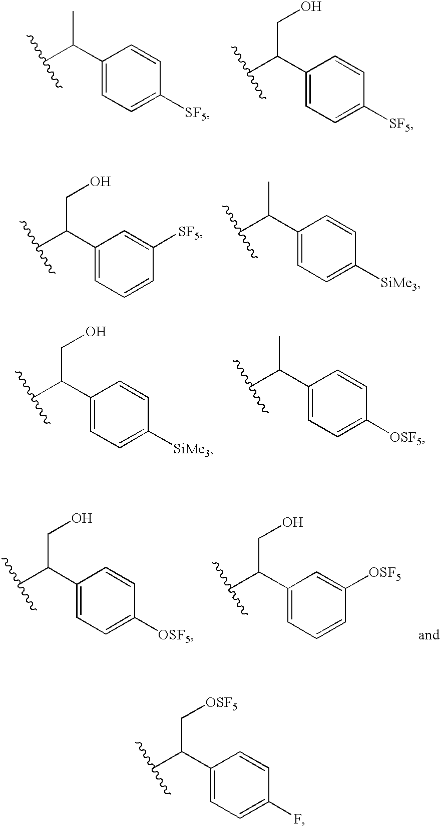

and
the R⁹-R¹⁰-moiety is:

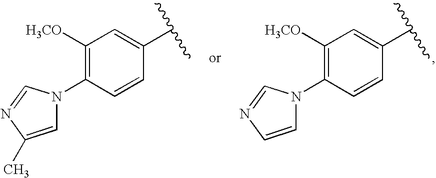

and
X is —N(R¹⁴)— and the compound of formula (I) is selected from the group consisting of: IB, ID, IF, IH, and IJ.

In another embodiment of this invention R¹ is selected from the group consisting of:

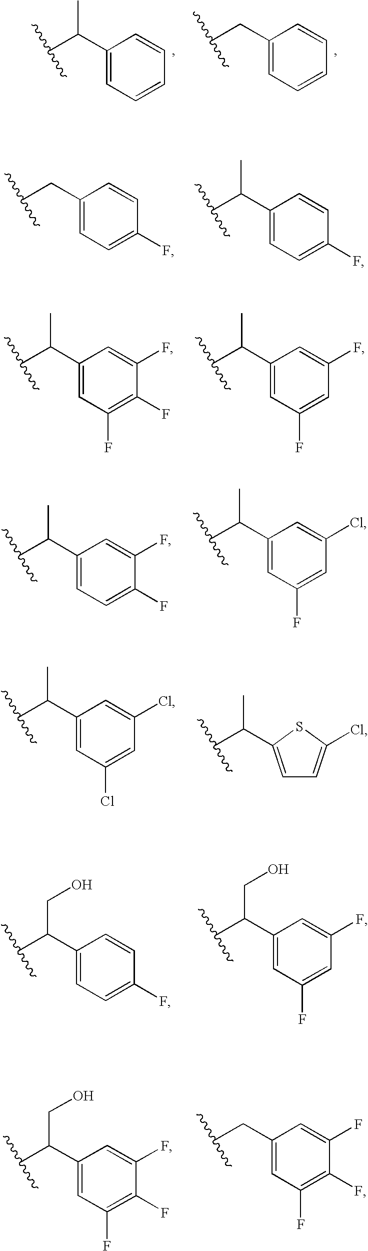

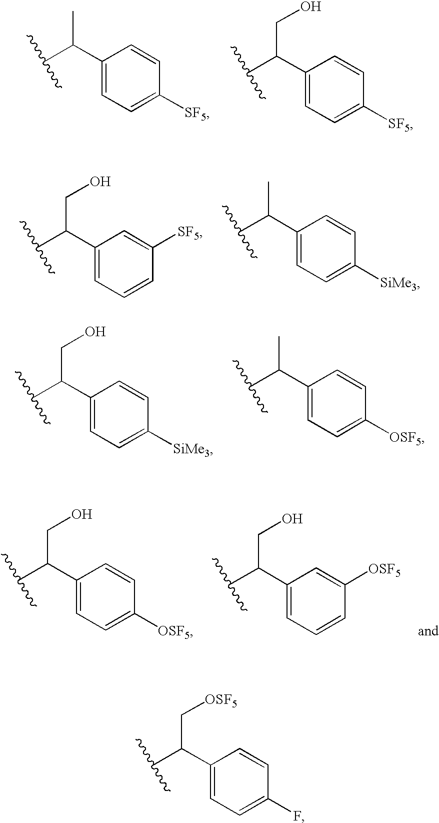

and
the R⁹-R¹⁰-moiety is:

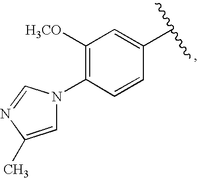

and
X is —N(R¹⁴)— and the compound of formula (I) is selected from the group consisting of: IB, ID, IF, IH, and IJ.

In another embodiment of this invention R¹ is selected from the group consisting of:

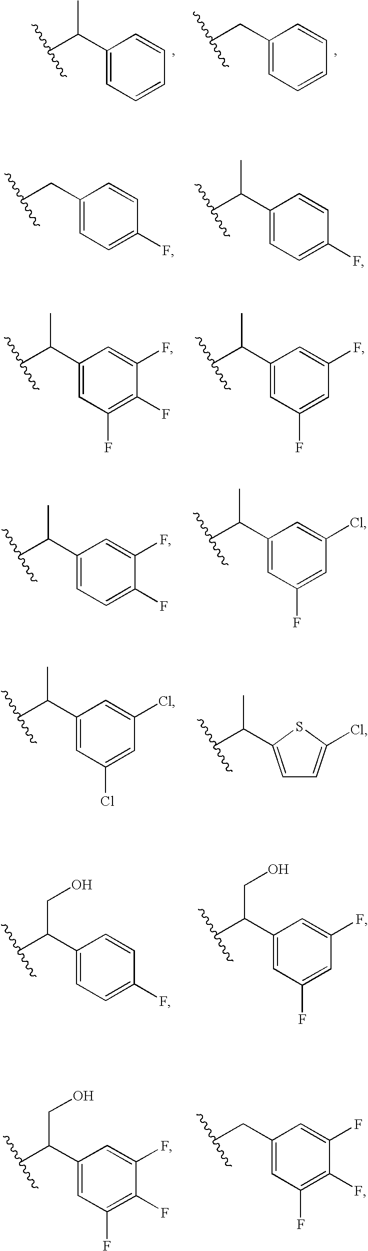

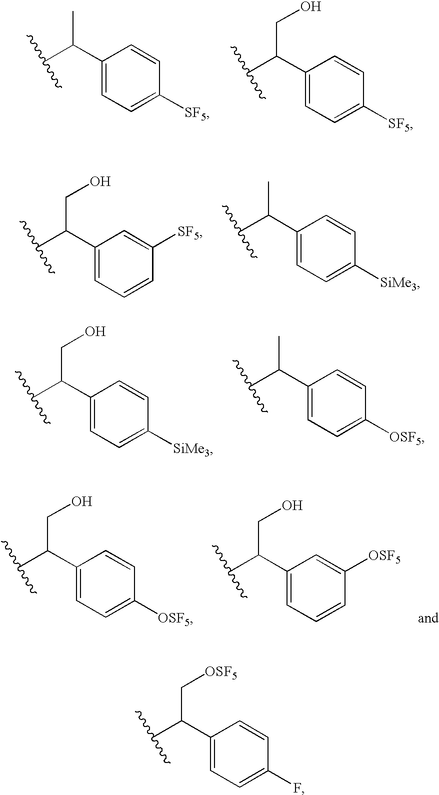

and
the R⁹-R¹⁰-moiety is:

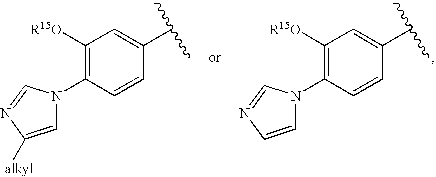

and
X is —N═ and the compound of formula (I) is selected from the group consisting of: IA, IC, IE, IG, II, and IK.

In another embodiment of this invention R¹ is selected from the group consisting of:

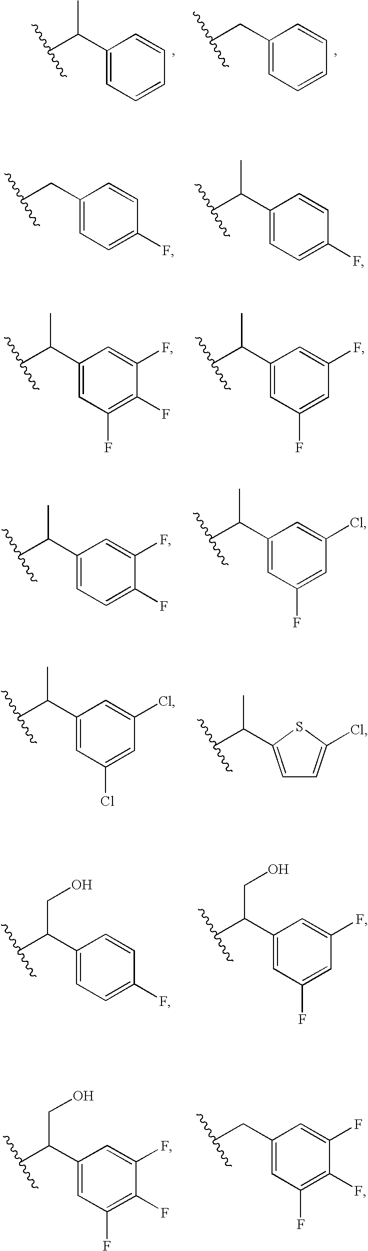

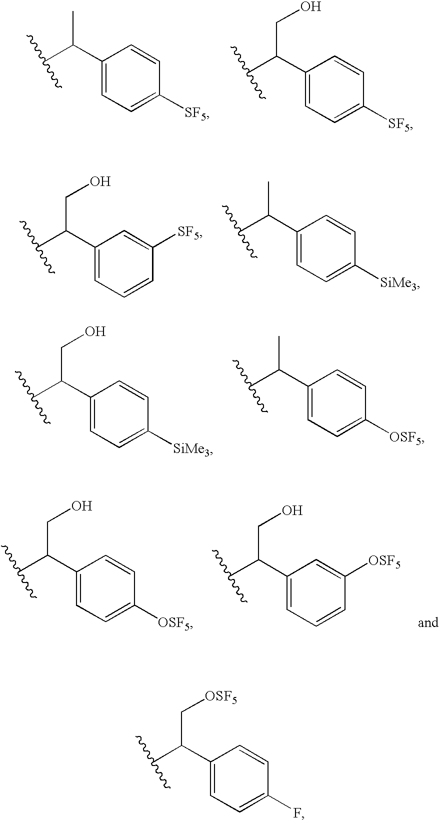

and
the R⁹-R¹⁰-moiety is:

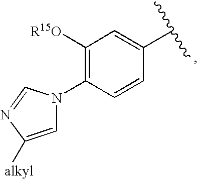

and
X is —N═ and the compound of formula (I) is selected from the group consisting of: IA, IC, IE, IG, II, and IK.

In another embodiment of this invention R¹ is selected from the group consisting of:

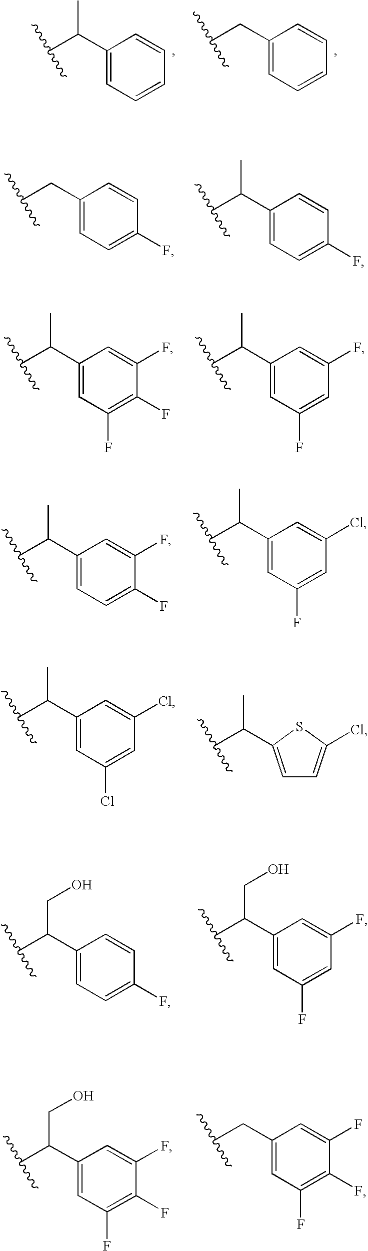

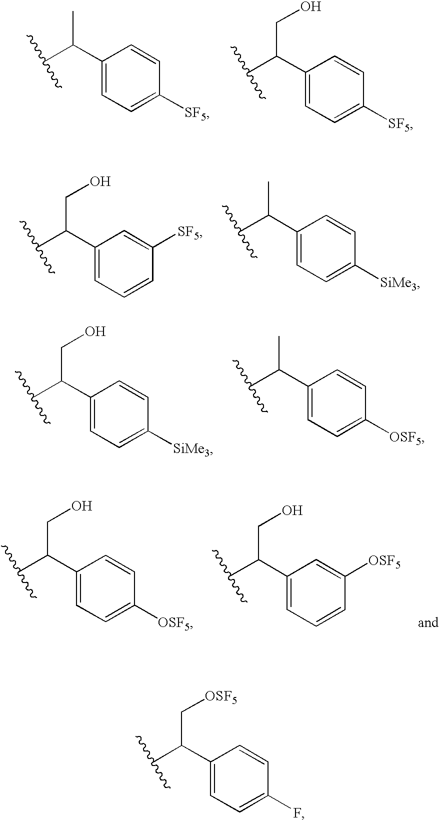

and
the R⁹-R¹⁰-moiety is:

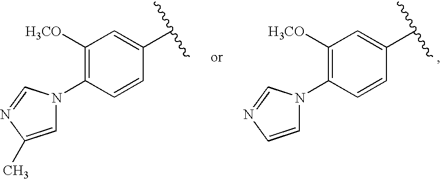

and
X is —N═ and the compound of formula (I) is selected from the group consisting of: IA, IC, IE, IG, II, and IK.

In another embodiment of this invention R¹ is selected from the group consisting of:

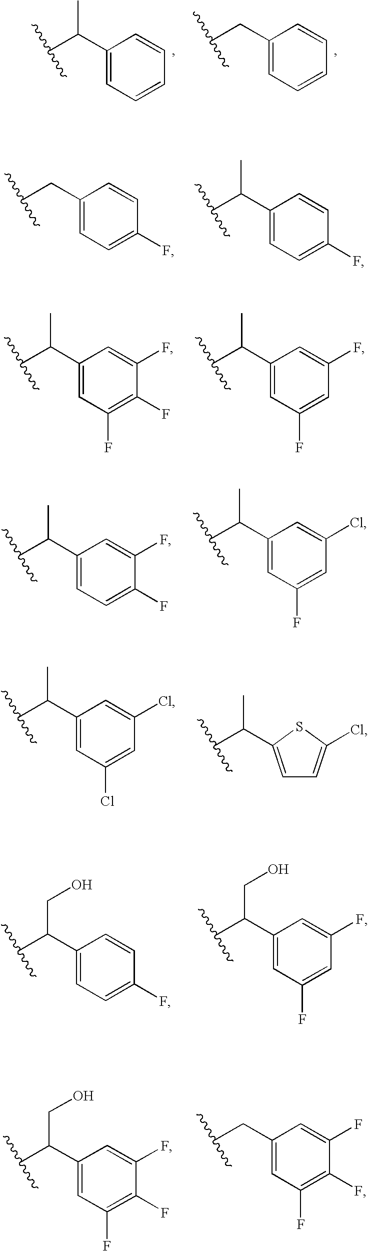

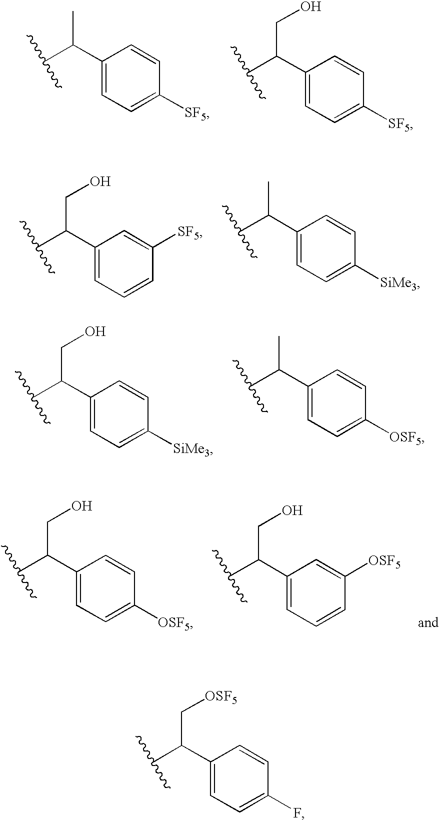

and
the R⁹-R¹⁰-moiety is:

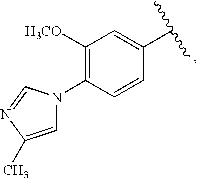

and
X is —N═ and the compound of formula (I) is selected from the group consisting of: IA, IC, IE, IG, II, and IK.

In another embodiment of this invention R¹ is selected from the group consisting of:

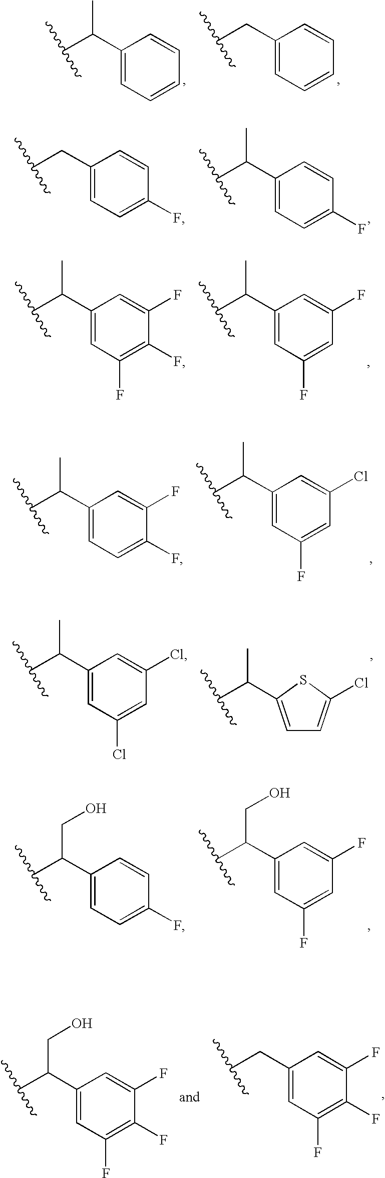

and
the R⁹-R¹⁰-moiety is:

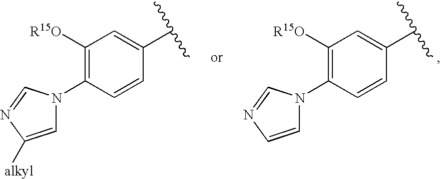

and
X is —N(R¹⁴)— and the compound of formula (I) is selected from the group consisting of: IB, ID, IF, IH, and IJ. In another embodiment of this invention R¹ is selected from the group consisting of:

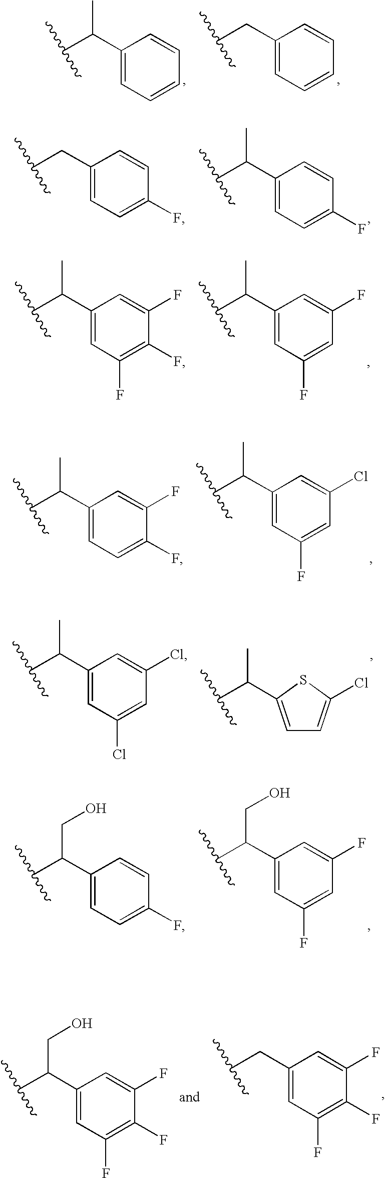

and
the R⁹-R¹⁰-moiety is:

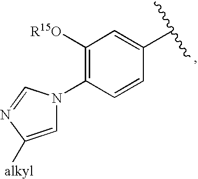

and
X is —N(R¹⁴)— and the compound of formula (I) is selected from the group consisting of: IB, ID, IF, IH, and IJ.

In another embodiment of this invention R¹ is selected from the group consisting of:

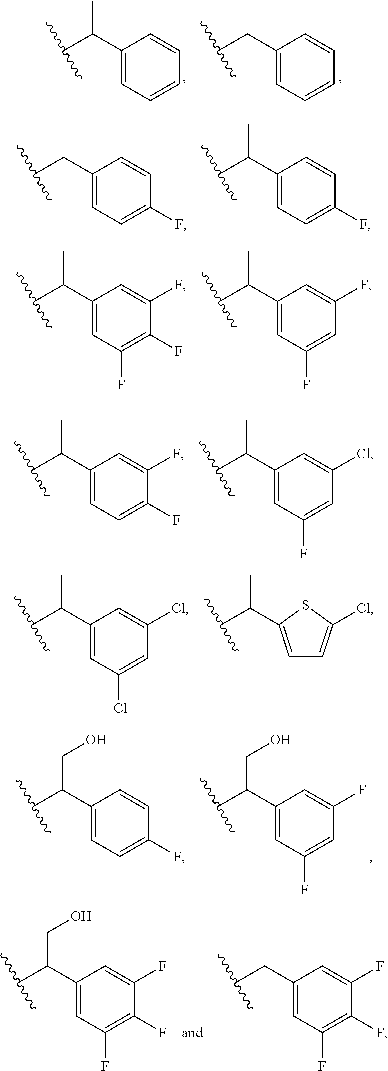

and
the R⁹-R¹⁰-moiety is:

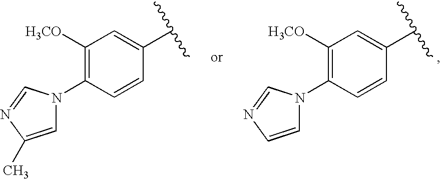

and
X is —N(R¹⁴)— and the compound of formula (I) is selected from the group consisting of: IB, ID, IF, IH, and IJ.

In another embodiment of this invention R¹ is selected from the group consisting of:

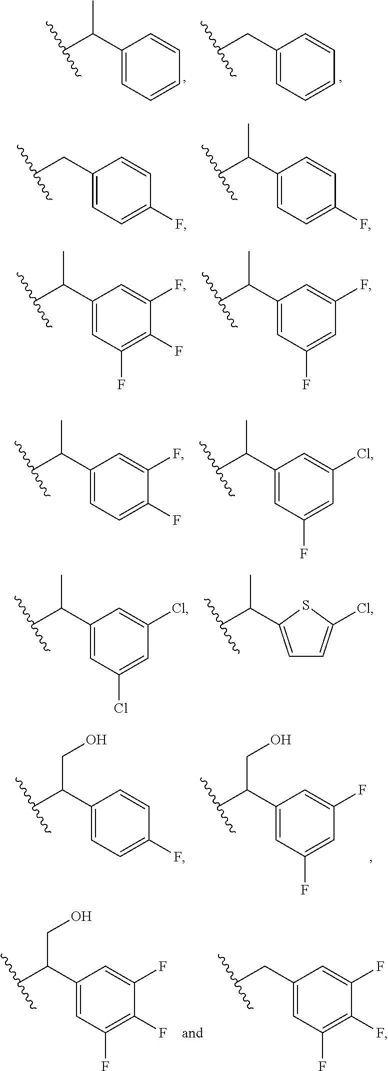

and
the R⁹-R¹⁰-moiety is:

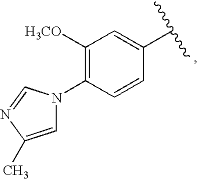

and
X is —N(R¹⁴)— and the compound of formula (I) is selected from the group consisting of: IB, ID, IF, IH, and IJ.

In another embodiment of this invention R¹ is selected from the group consisting of:

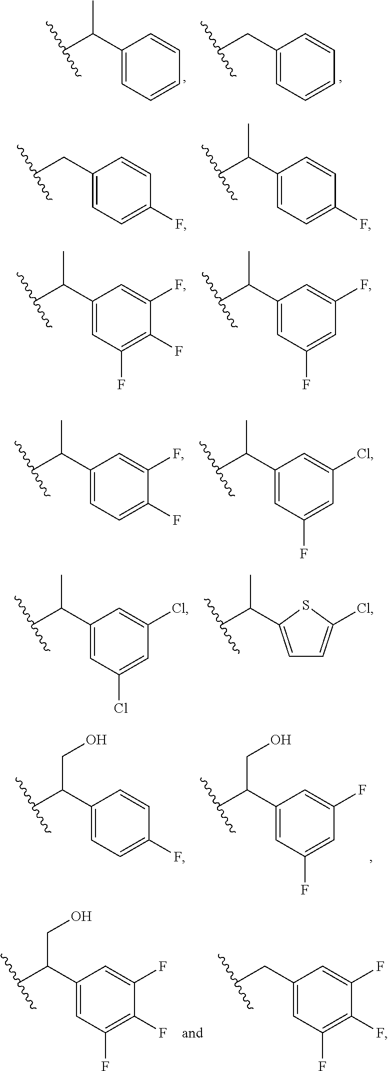

and
the R⁹-R¹⁰-moiety is:

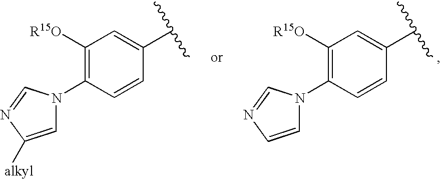

and
X is —N═ and the compound of formula (I) is selected from the group consisting of: IA, IC, IE, IG, II, and IK.

In another embodiment of this invention R¹ is selected from the group consisting of:

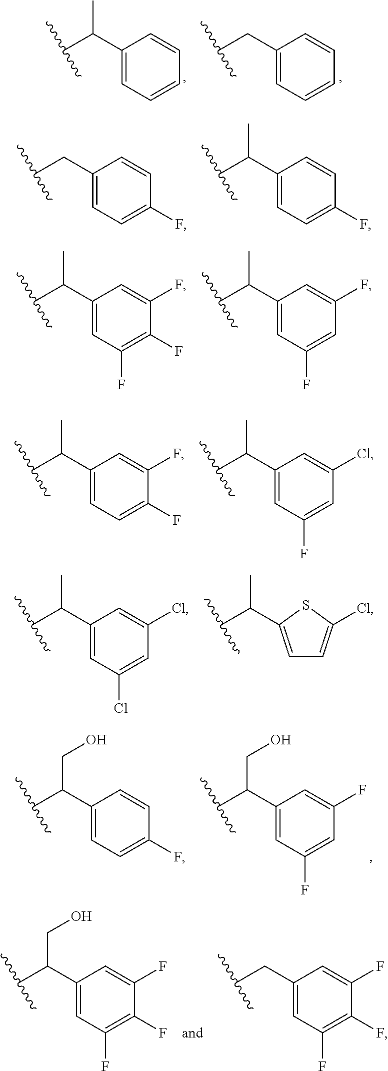

and
the R⁹-R¹⁰-moiety is:

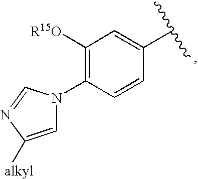

and
X is —N═ and the compound of formula (I) is selected from the group consisting of: IA, IC, IE, IG, II, and IK.

In another embodiment of this invention R¹ is selected from the group consisting of:

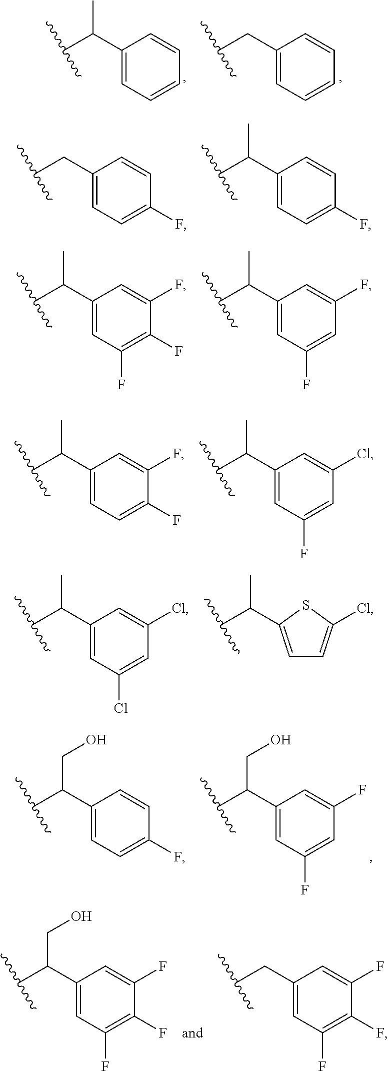

and
the R⁹-R¹⁰-moiety is:

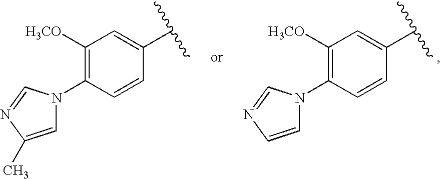

and
X is —N═ and the compound of formula (I) is selected from the group consisting of: IA, IC, IE, IG, II, and IK.

In another embodiment of this invention R¹ is selected from the group consisting of:

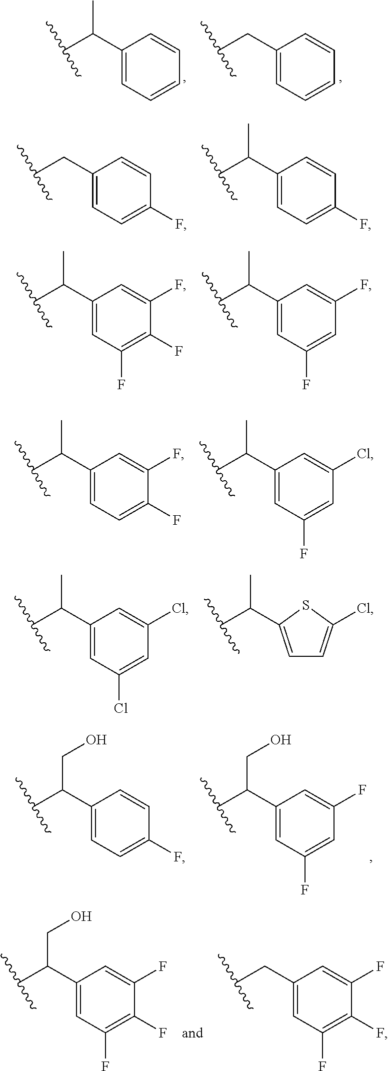

and
the R⁹-R¹⁰-moiety is:

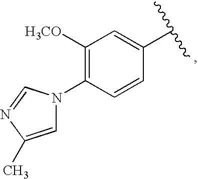

and
X is —N═ and the compound of formula (I) is selected from the group consisting of: IA, IC, IE, IG, II, and IK.

In another embodiment of this invention R¹ is selected from the group consisting of:

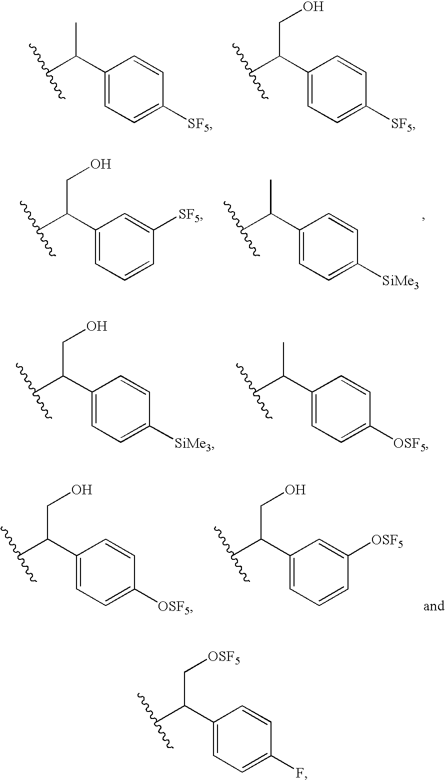

and

the R⁹-R¹⁰-moiety is:

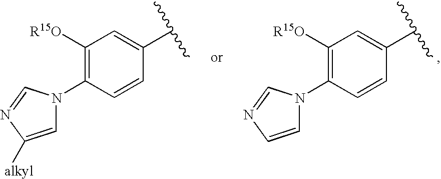

and
X is —N(R¹⁴)— and the compound of formula (I) is selected from the group consisting of: IB, ID, IF, IH, and IJ.

In another embodiment of this invention R¹ is selected from the group consisting of:

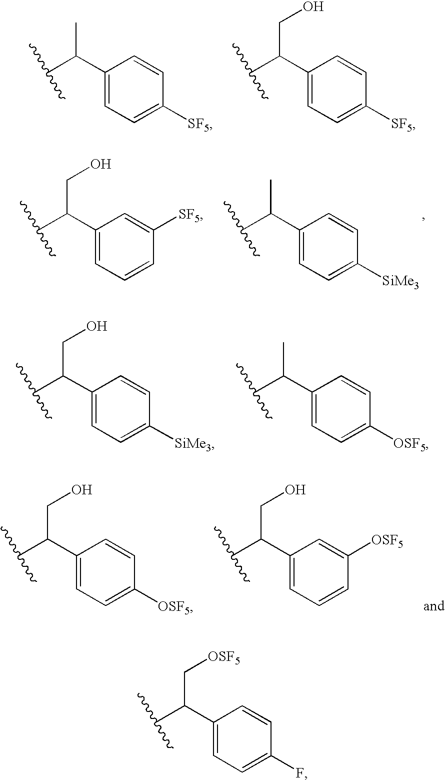

and
the R⁹-R¹⁰-moiety is:

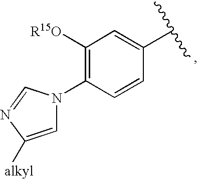

and
X is —N(R¹⁴)— and the compound of formula (I) is selected from the group consisting of: IB, ID, IF, IH, and IJ.

In another embodiment of this invention R¹ is selected from the group consisting of:

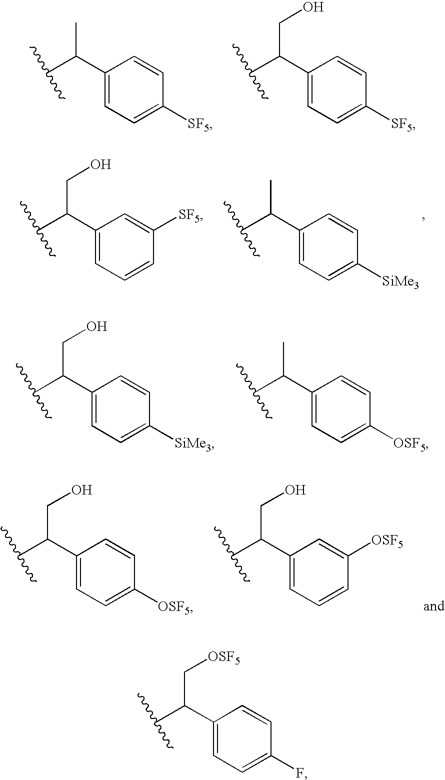

and
the R⁹-R¹⁰-moiety is:

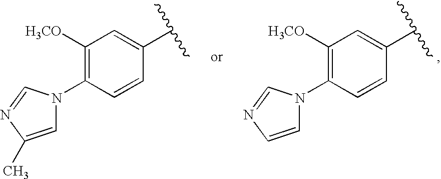

and
X is —N(R¹⁴)— and the compound of formula (I) is selected from the group consisting of: IB, ID, IF, IH, and IJ.

In another embodiment of this invention R¹ is selected from the group consisting of:

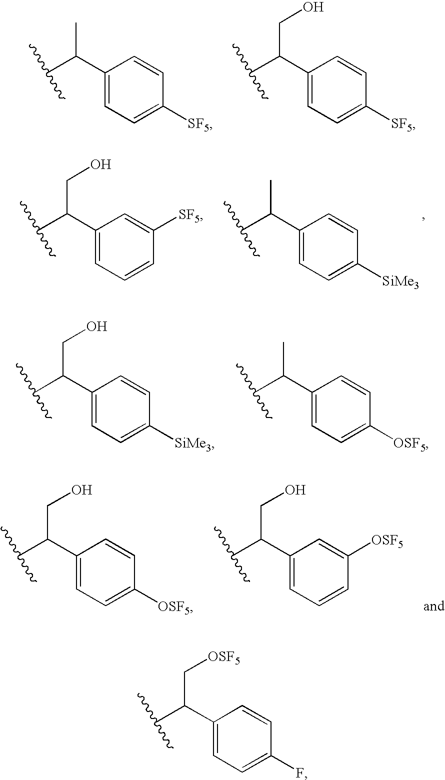

and
the R⁹-R¹⁰-moiety is:

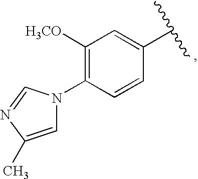

and
X is —N(R¹⁴)— and the compound of formula (I) is selected from the group consisting of: IB, ID, IF, IH, and IJ.

In another embodiment of this invention R¹ is selected from the group consisting of:

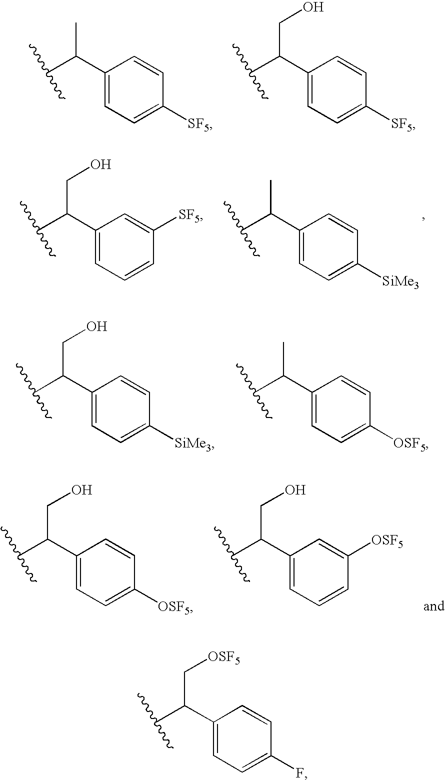

and
the R⁹-R¹⁰-moiety is:

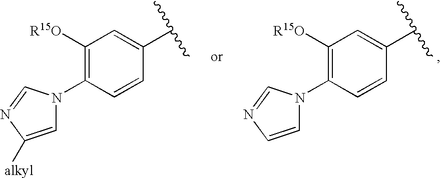

and
X is —N═ and the compound of formula (I) is selected from the group consisting of: IA, IC, IE, IG, II, and IK.

In another embodiment of this invention R¹ is selected from the group consisting of:

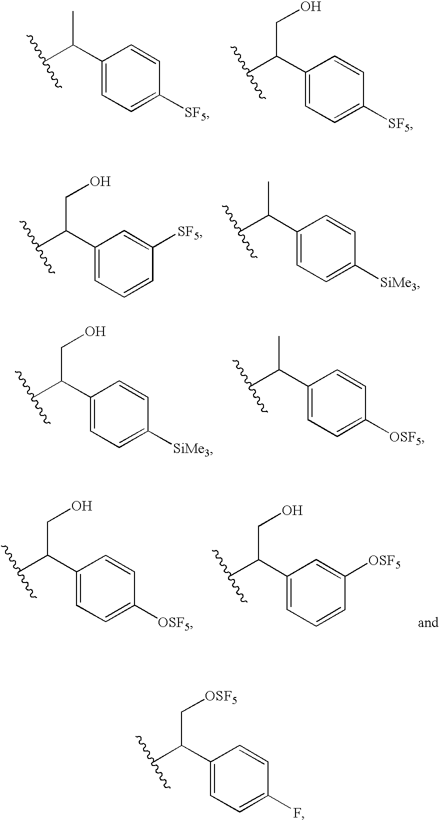

and
the R⁹-R¹⁰-moiety is:

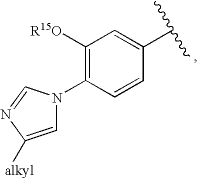

and
X is —N═ and the compound of formula (I) is selected from the group consisting of: IA, IC, IE, IG, II, and IK.

In another embodiment of this invention R¹ is selected from the group consisting of:

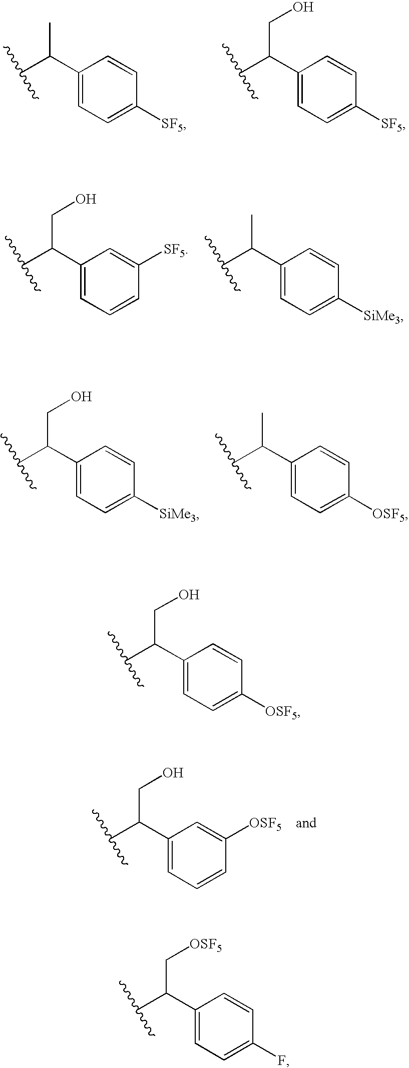

and
the R⁹-R¹⁰-moiety is:

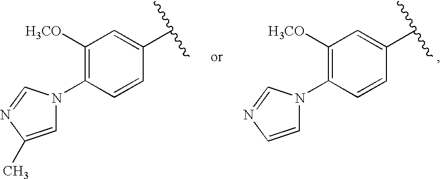

and
X is —N═ and the compound of formula (I) is selected from the group consisting of: IA, IC, IE, IG, II, and IK.

In another embodiment of this invention R¹ is selected from the group consisting of:

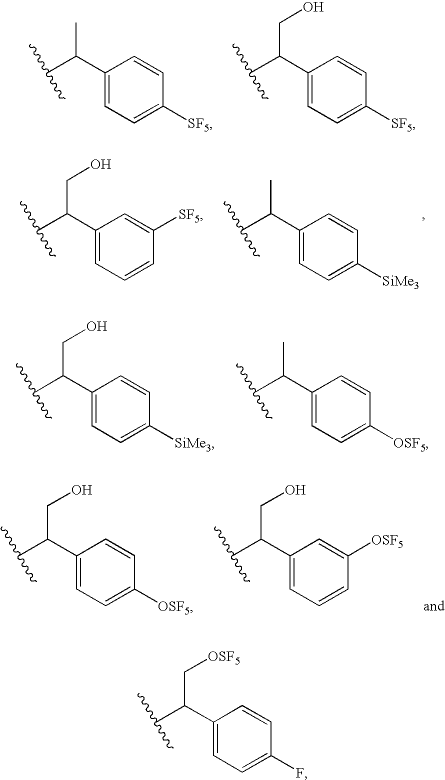

and

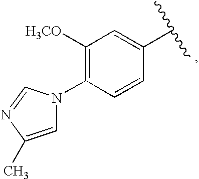

and
X is —N═ and the compound of formula (I) is selected from the group consisting of: IA, IC, IE, IG, II, and IK.

Representative compounds of the invention include, but are not limited to:

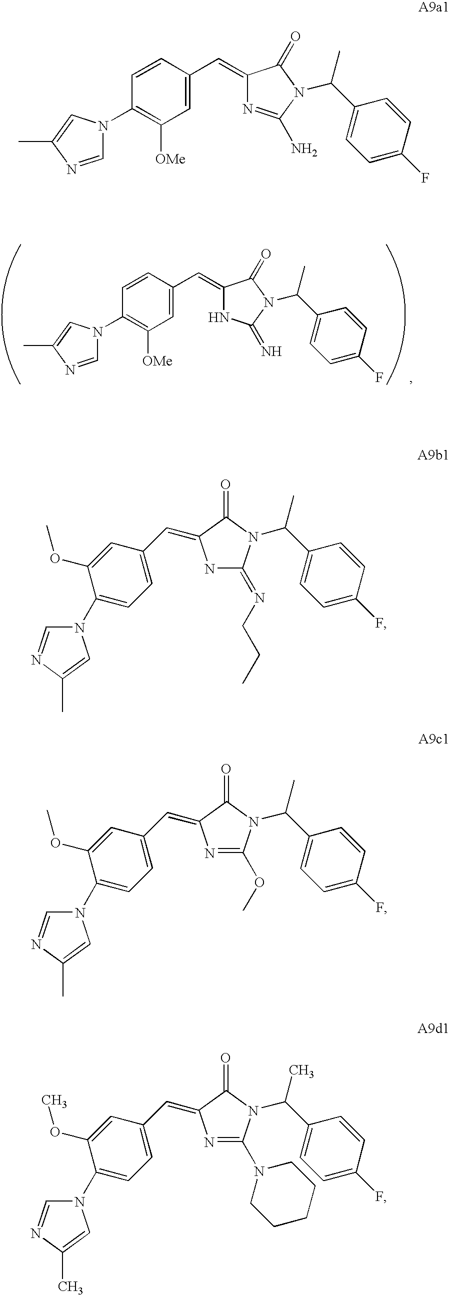

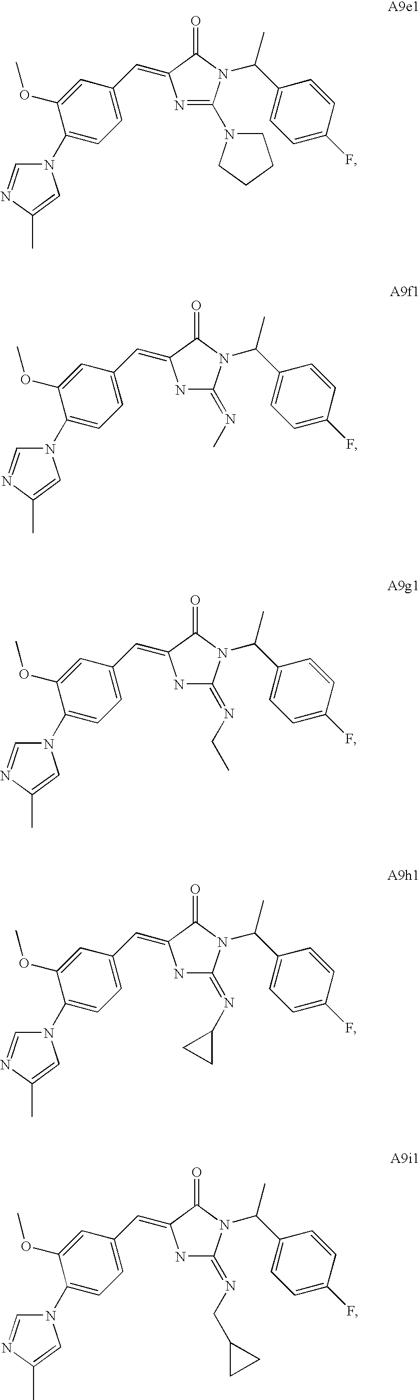

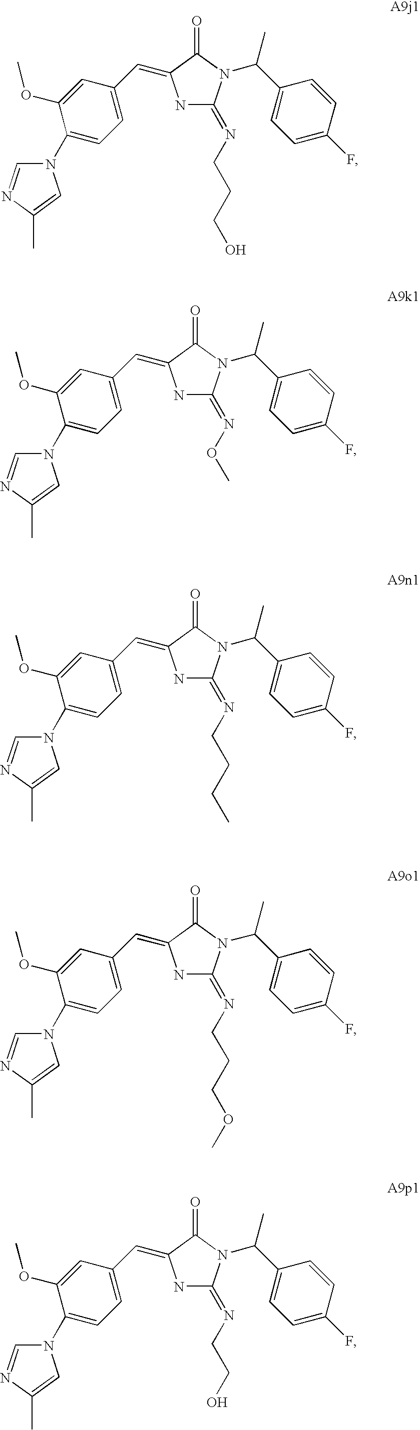

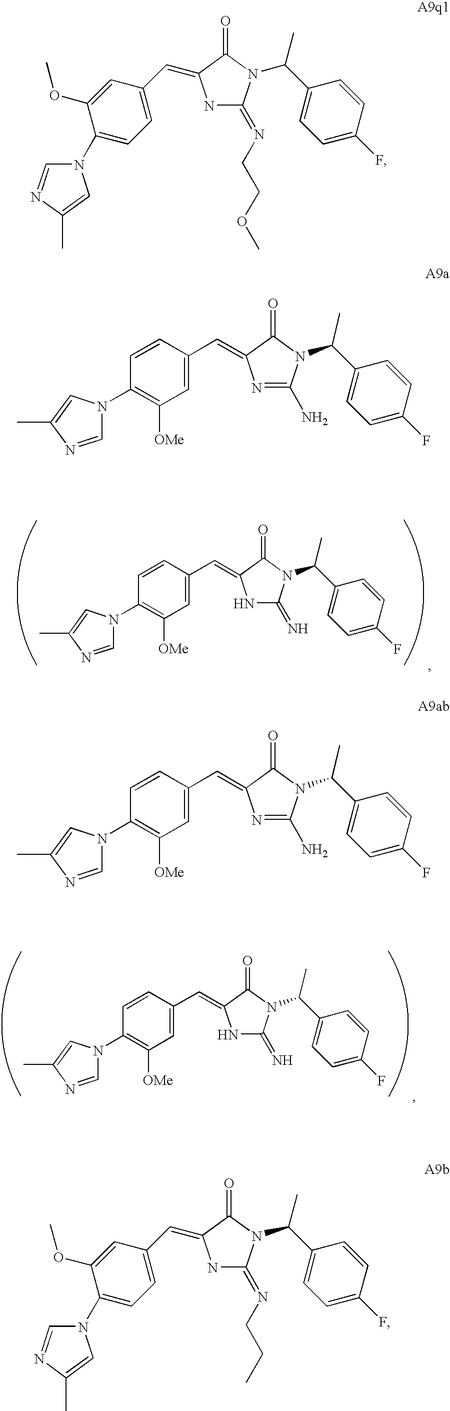

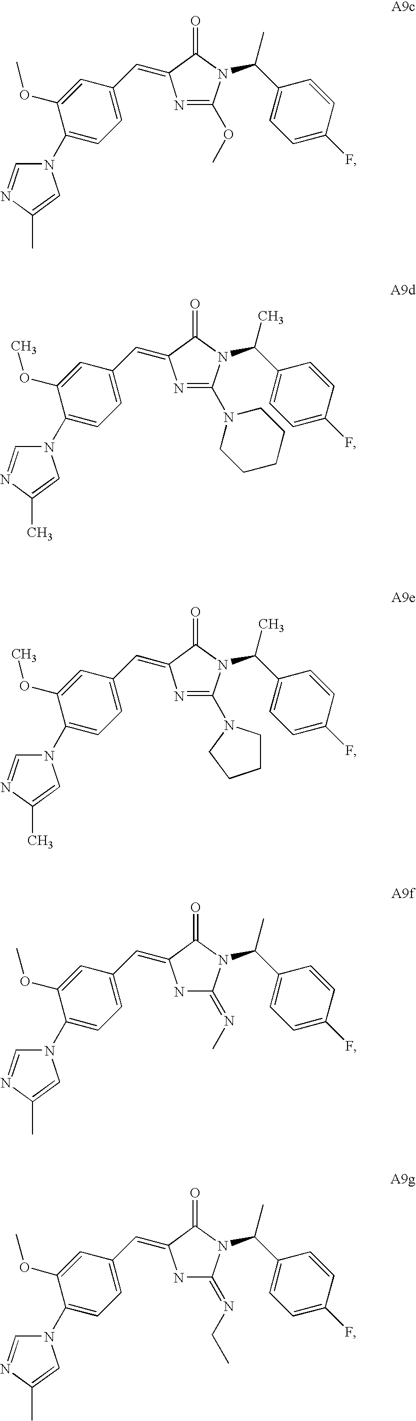

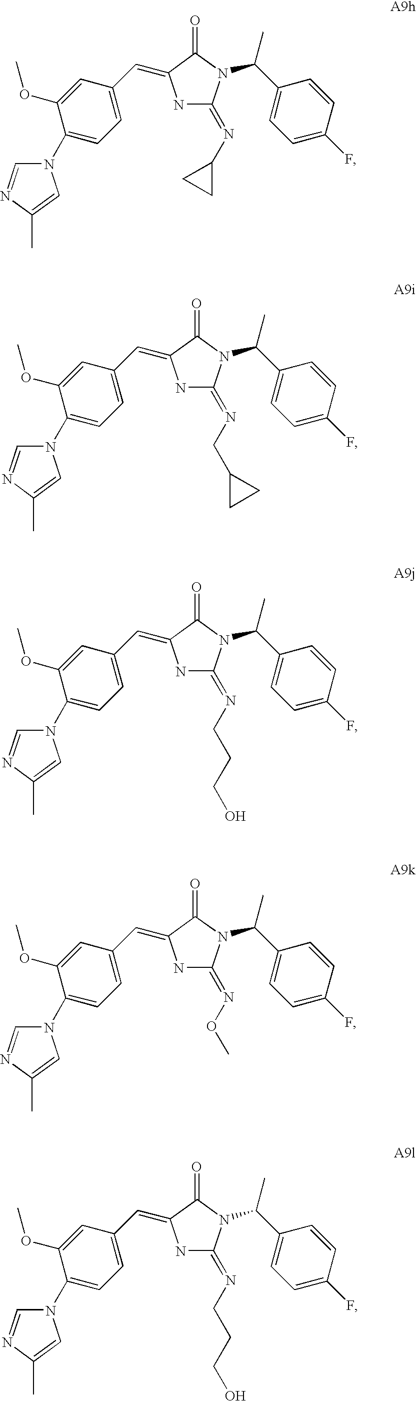

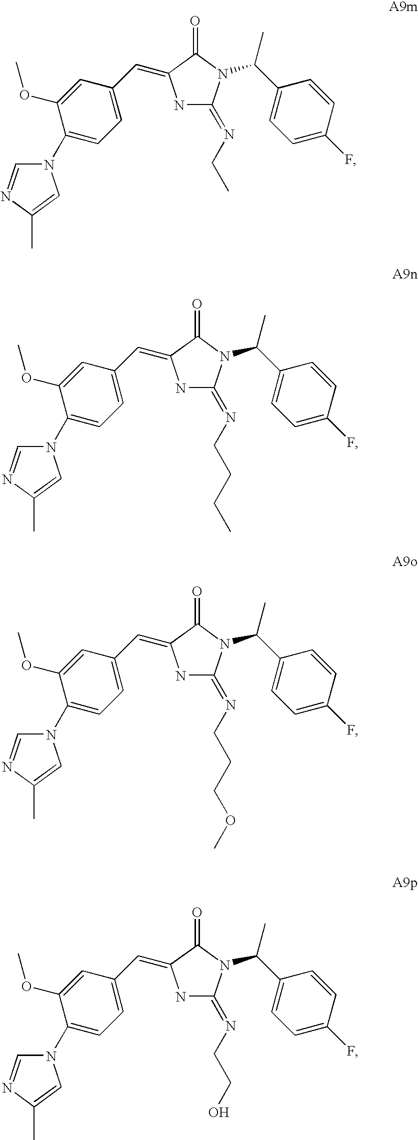

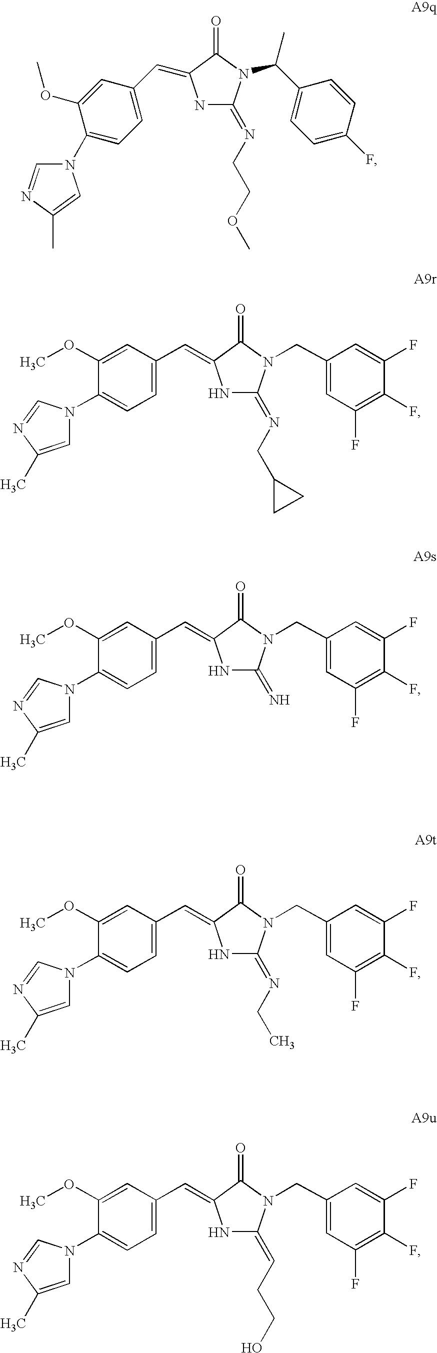

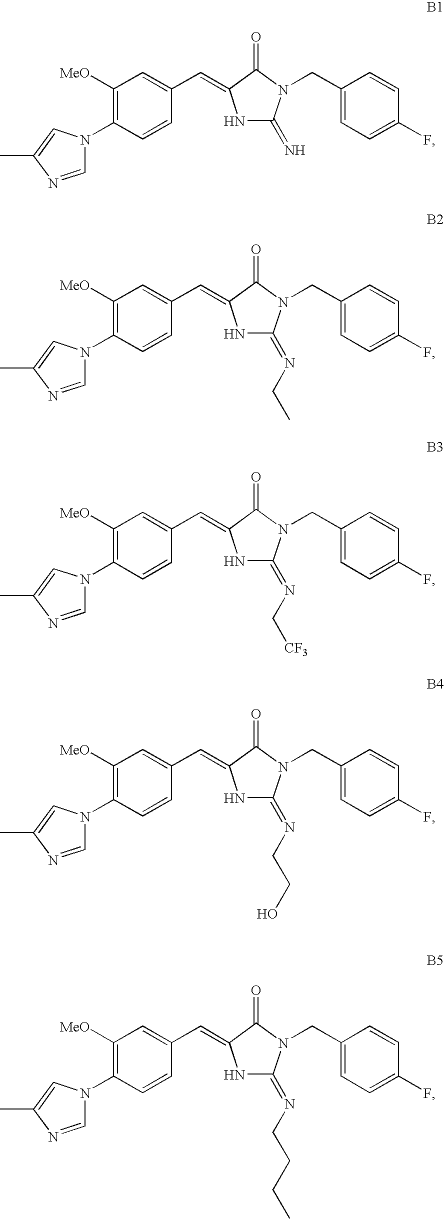

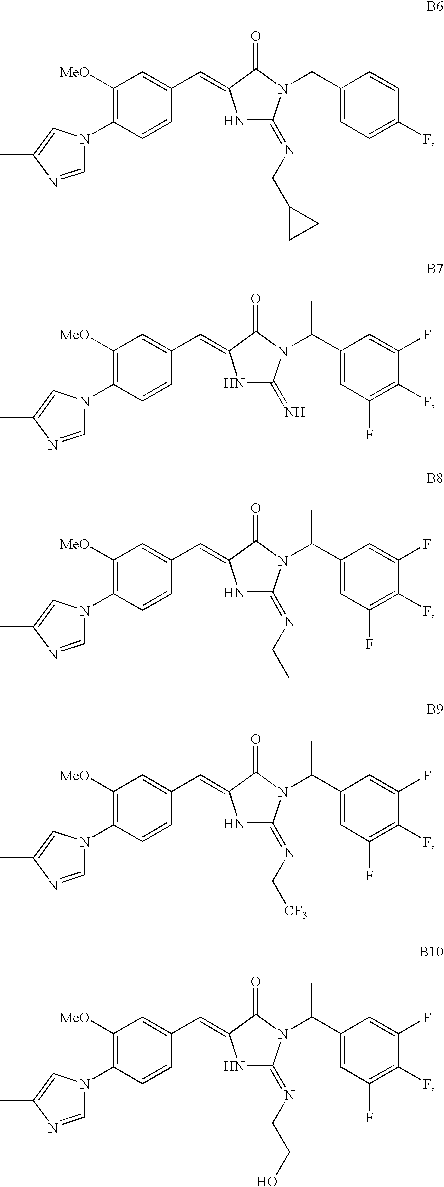

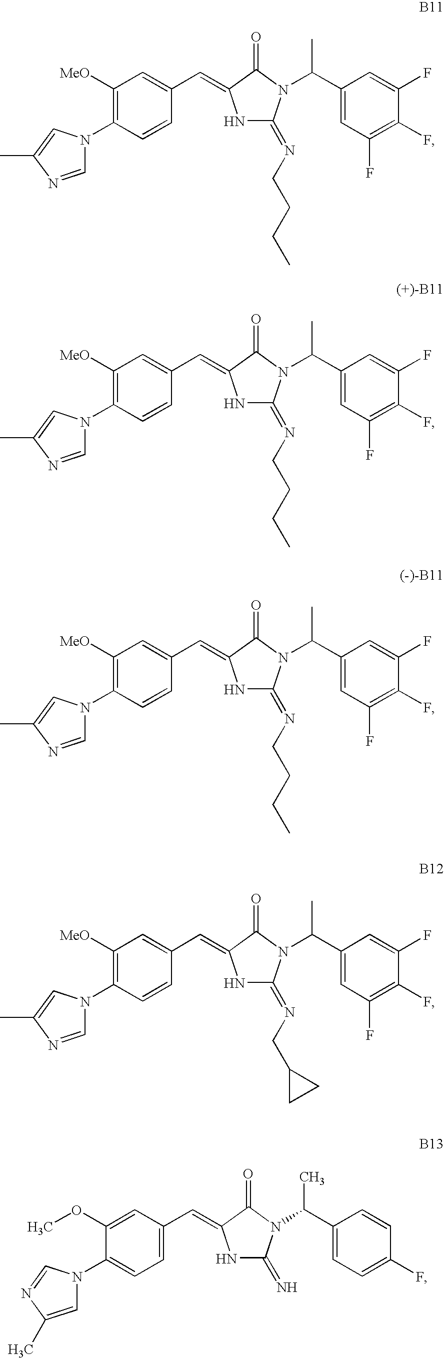

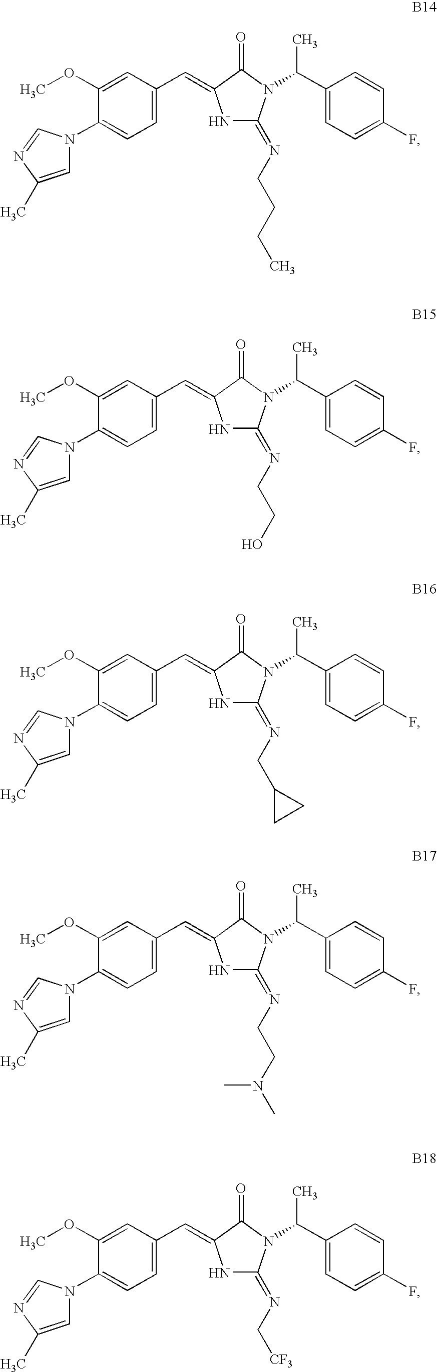

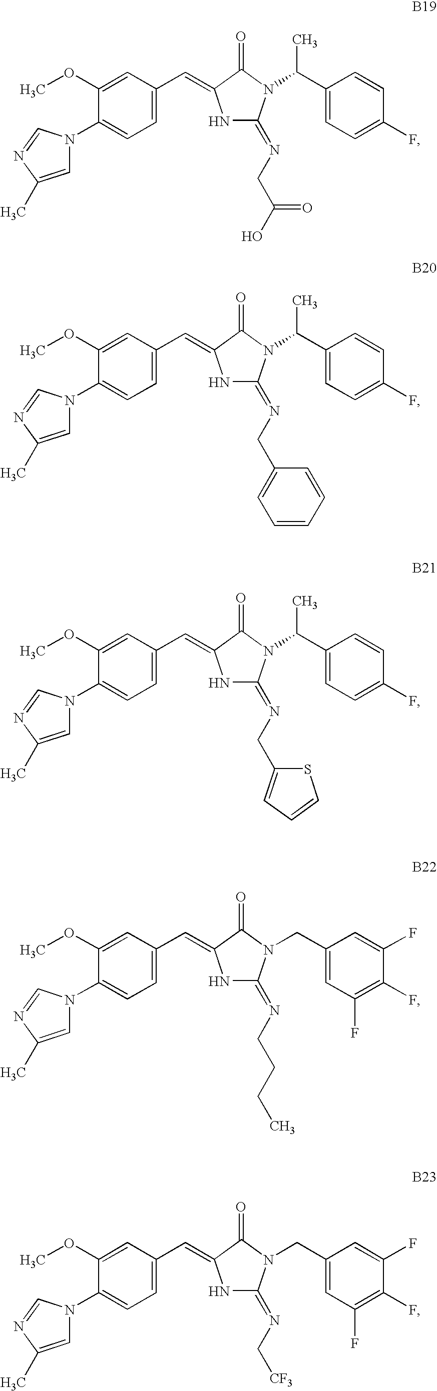

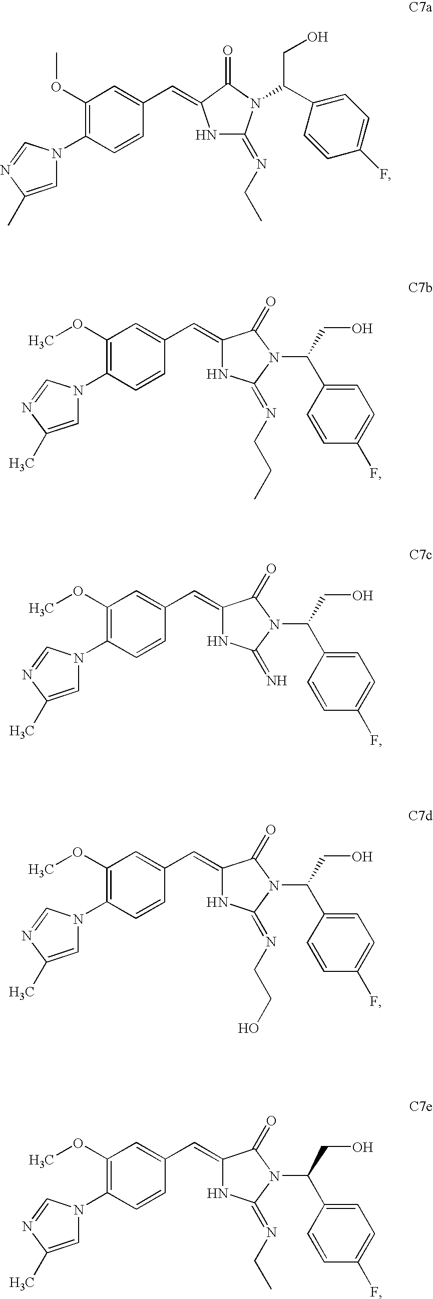

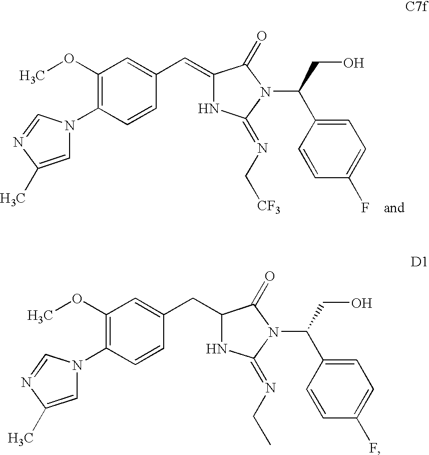

Representative compounds of the invention include, but are not limited to:

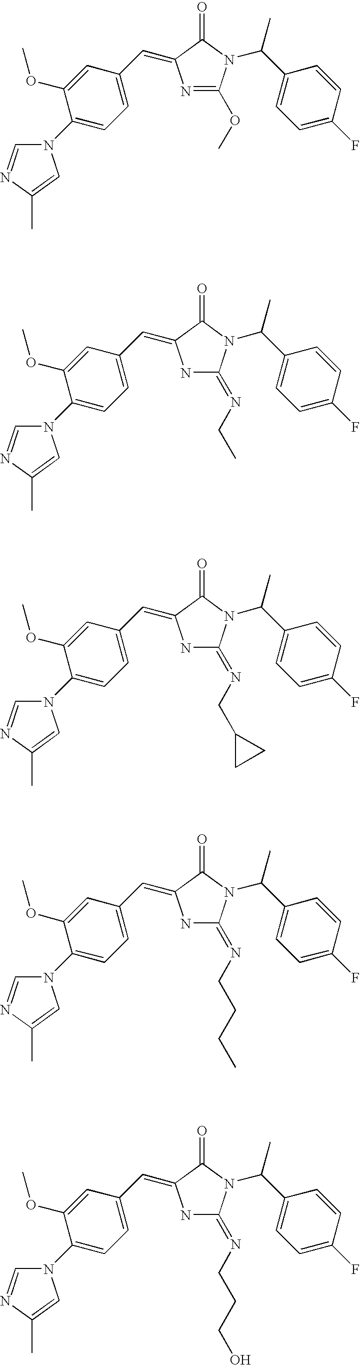

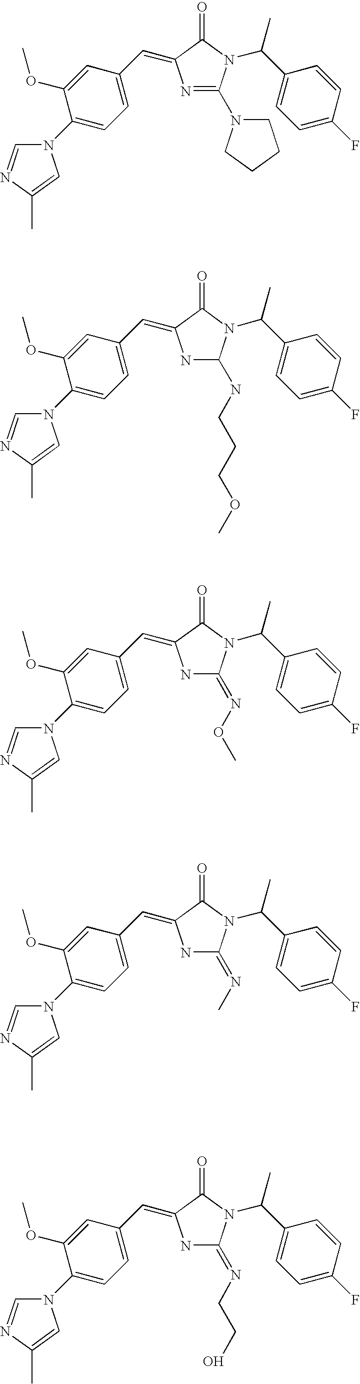

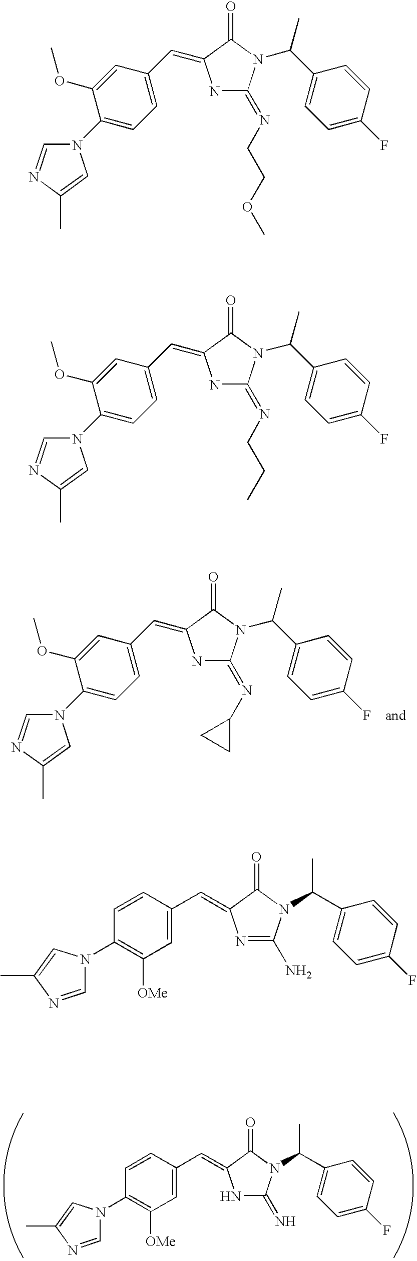

or a pharmaceutically acceptable salt, solvate, ester or prodrug thereof.

Representative compounds of the invention include, but are not limited to:

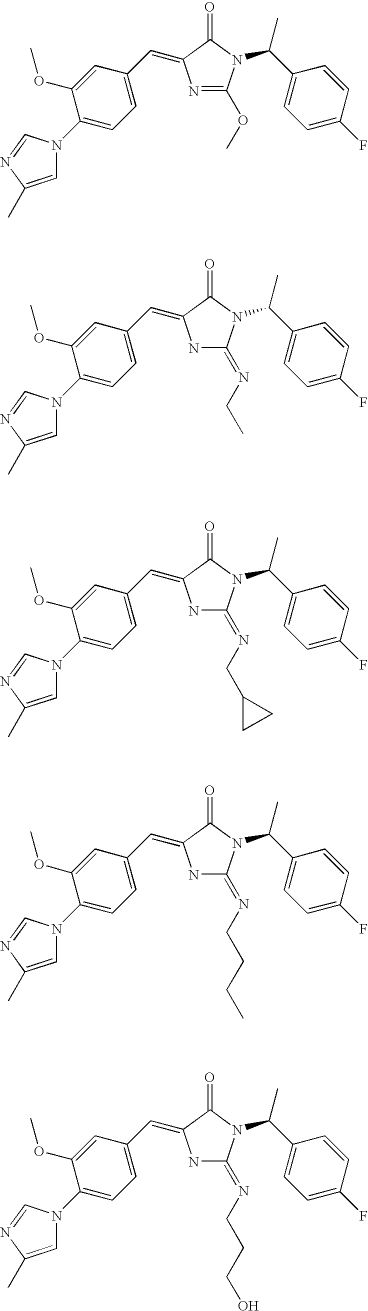

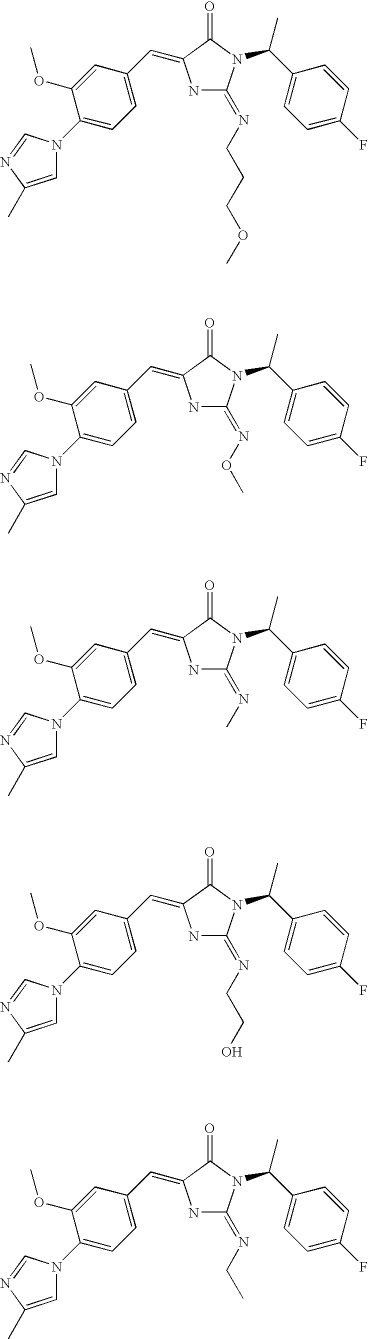

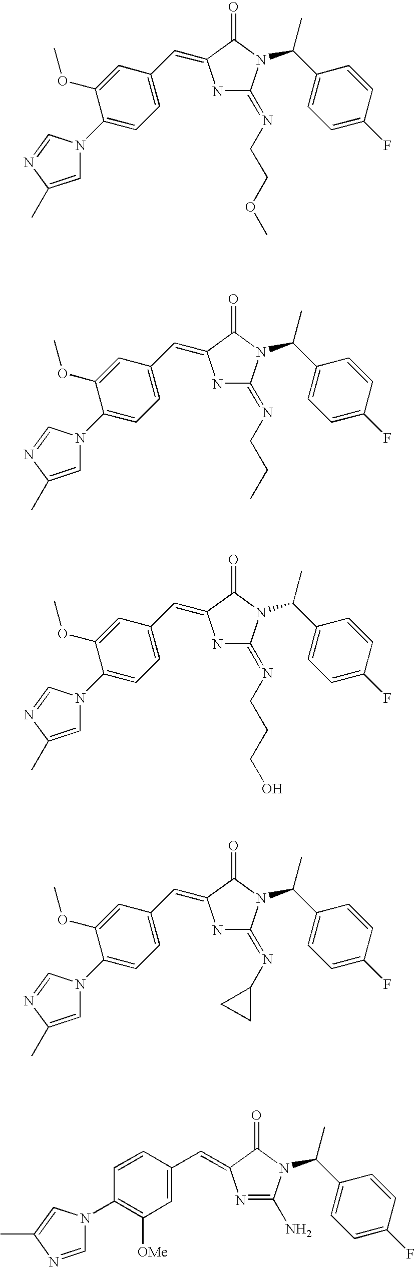

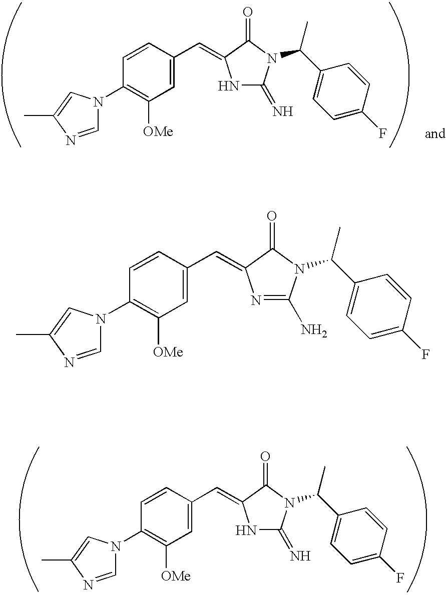

or a pharmaceutically acceptable salt, solvate, ester or prodrug thereof.

Another embodiment of this invention is directed to a compound of formula A9a1, or a pharmaceutically acceptable salt, ester or solvate thereof.

Another embodiment of this invention is directed to a compound of formula A9b1, or a pharmaceutically acceptable salt, ester or solvate thereof.

Another embodiment of this invention is directed to a compound of formula A9c1, or a pharmaceutically acceptable salt, ester or solvate thereof.

Another embodiment of this invention is directed to a compound of formula A9d1, or a pharmaceutically acceptable salt, ester or solvate thereof.

Another embodiment of this invention is directed to a compound of formula A9e1, or a pharmaceutically acceptable salt, ester or solvate thereof.

Another embodiment of this invention is directed to a compound of formula A9f1, or a pharmaceutically acceptable salt, ester or solvate thereof.

Another embodiment of this invention is directed to a compound of formula A9g1, or a pharmaceutically acceptable salt, ester or solvate thereof.

Another embodiment of this invention is directed to a compound of formula A9h1, or a pharmaceutically acceptable salt, ester or solvate thereof.

Another embodiment of this invention is directed to a compound of formula A9i1, or a pharmaceutically acceptable salt, ester or solvate thereof.

Another embodiment of this invention is directed to a compound of formula A9j1, or a pharmaceutically acceptable salt, ester or solvate thereof.

Another embodiment of this invention is directed to a compound of formula A9k1, or a pharmaceutically acceptable salt, ester or solvate thereof.

Another embodiment of this invention is directed to a compound of formula A9n1, or a pharmaceutically acceptable salt, ester or solvate thereof.

Another embodiment of this invention is directed to a compound of formula A9o1, or a pharmaceutically acceptable salt, ester or solvate thereof.

Another embodiment of this invention is directed to a compound of formula A9 μl, or a pharmaceutically acceptable salt, ester or solvate thereof.

Another embodiment of this invention is directed to a compound of formula A9q1, or a pharmaceutically acceptable salt, ester or solvate thereof.

Another embodiment of this invention is directed to a compound of formula A9a, or a pharmaceutically acceptable salt, ester or solvate thereof.

Another embodiment of this invention is directed to a compound of formula A9b, or a pharmaceutically acceptable salt, ester or solvate thereof.

Another embodiment of this invention is directed to a compound of formula A9c, or a pharmaceutically acceptable salt, ester or solvate thereof.

Another embodiment of this invention is directed to a compound of formula A9d, or a pharmaceutically acceptable salt, ester or solvate thereof.

Another embodiment of this invention is directed to a compound of formula A9e, or a pharmaceutically acceptable salt, ester or solvate thereof.

Another embodiment of this invention is directed to a compound of formula A9f, or a pharmaceutically acceptable salt, ester or solvate thereof.

Another embodiment of this invention is directed to a compound of formula A9g, or a pharmaceutically acceptable salt, ester or solvate thereof.

Another embodiment of this invention is directed to a compound of formula A9h, or a pharmaceutically acceptable salt, ester or solvate thereof.

Another embodiment of this invention is directed to a compound of formula A91, or a pharmaceutically acceptable salt, ester or solvate thereof.

Another embodiment of this invention is directed to a compound of formula A9j, or a pharmaceutically acceptable salt, ester or solvate thereof.

Another embodiment of this invention is directed to a compound of formula A9k, or a pharmaceutically acceptable salt, ester or solvate thereof.

Another embodiment of this invention is directed to a compound of formula A9I, or a pharmaceutically acceptable salt, ester or solvate thereof.

Another embodiment of this invention is directed to a compound of formula A9m, or a pharmaceutically acceptable salt, ester or solvate thereof.

Another embodiment of this invention is directed to a compound of formula A9n, or a pharmaceutically acceptable salt, ester or solvate thereof.

Another embodiment of this invention is directed to a compound of formula A9o, or a pharmaceutically acceptable salt, ester or solvate thereof.

Another embodiment of this invention is directed to a compound of formula A9p, or a pharmaceutically acceptable salt, ester or solvate thereof.

Another embodiment of this invention is directed to a compound of formula A9q, or a pharmaceutically acceptable salt, ester or solvate thereof.

Another embodiment of this invention is directed to a compound of formula A9r, or a pharmaceutically acceptable salt, ester or solvate thereof.

Another embodiment of this invention is directed to a compound of formula A9s, or a pharmaceutically acceptable salt, ester or solvate thereof.

Another embodiment of this invention is directed to a compound of formula A9t, or a pharmaceutically acceptable salt, ester or solvate thereof.

Another embodiment of this invention is directed to a compound of formula A9u, or a pharmaceutically acceptable salt, ester or solvate thereof.

Another embodiment of this invention is directed to a compound of formula A9ab, or a pharmaceutically acceptable salt, ester or solvate thereof.

Another embodiment of this invention is directed to a compound of formula B1, or a pharmaceutically acceptable salt, ester or solvate thereof.

Another embodiment of this invention is directed to a compound of formula B2, or a pharmaceutically acceptable salt, ester or solvate thereof.

Another embodiment of this invention is directed to a compound of formula B3, or a pharmaceutically acceptable salt, ester or solvate thereof.

Another embodiment of this invention is directed to a compound of formula B4, or a pharmaceutically acceptable salt, ester or solvate thereof.

Another embodiment of this invention is directed to a compound of formula B5, or a pharmaceutically acceptable salt, ester or solvate thereof.

Another embodiment of this invention is directed to a compound of formula B6, or a pharmaceutically acceptable salt, ester or solvate thereof.

Another embodiment of this invention is directed to a compound of formula B7, or a pharmaceutically acceptable salt, ester or solvate thereof.

Another embodiment of this invention is directed to a compound of formula B8, or a pharmaceutically acceptable salt, ester or solvate thereof.

Another embodiment of this invention is directed to a compound of formula B9, or a pharmaceutically acceptable salt, ester or solvate thereof.

Another embodiment of this invention is directed to a compound of formula B10, or a pharmaceutically acceptable salt, ester or solvate thereof.

Another embodiment of this invention is directed to a compound of formula B11, or a pharmaceutically acceptable salt, ester or solvate thereof.

Another embodiment of this invention is directed to a compound of formula (+)-B11, or a pharmaceutically acceptable salt, ester or solvate thereof. Another embodiment of this invention is directed to a compound of formula (−)-B11, or a pharmaceutically acceptable salt, ester or solvate thereof.

Another embodiment of this invention is directed to a compound of formula B12, or a pharmaceutically acceptable salt, ester or solvate thereof.

Another embodiment of this invention is directed to a compound of formula B13, or a pharmaceutically acceptable salt, ester or solvate thereof.

Another embodiment of this invention is directed to a compound of formula B14, or a pharmaceutically acceptable salt, ester or solvate thereof.

Another embodiment of this invention is directed to a compound of formula B15, or a pharmaceutically acceptable salt, ester or solvate thereof.

Another embodiment of this invention is directed to a compound of formula B16, or a pharmaceutically acceptable salt, ester or solvate thereof.

Another embodiment of this invention is directed to a compound of formula B17, or a pharmaceutically acceptable salt, ester or solvate thereof.

Another embodiment of this invention is directed to a compound of formula B18, or a pharmaceutically acceptable salt, ester or solvate thereof.

Another embodiment of this invention is directed to a compound of formula B19, or a pharmaceutically acceptable salt, ester or solvate thereof.

Another embodiment of this invention is directed to a compound of formula B20, or a pharmaceutically acceptable salt, ester or solvate thereof.

Another embodiment of this invention is directed to a compound of formula B21, or a pharmaceutically acceptable salt, ester or solvate thereof.

Another embodiment of this invention is directed to a compound of formula B22, or a pharmaceutically acceptable salt, ester or solvate thereof.

Another embodiment of this invention is directed to a compound of formula B23, or a pharmaceutically acceptable salt, ester or solvate thereof.

Another embodiment of this invention is directed to a compound of formula C7a, or a pharmaceutically acceptable salt, ester or solvate thereof.

Another embodiment of this invention is directed to a compound of formula C7b, or a pharmaceutically acceptable salt, ester or solvate thereof.

Another embodiment of this invention is directed to a compound of formula C7c, or a pharmaceutically acceptable salt, ester or solvate thereof.

Another embodiment of this invention is directed to a compound of formula C7d, or a pharmaceutically acceptable salt, ester or solvate thereof.

Another embodiment of this invention is directed to a compound of formula C7e, or a pharmaceutically acceptable salt, ester or solvate thereof.

Another embodiment of this invention is directed to a compound of formula C7f, or a pharmaceutically acceptable salt, ester or solvate thereof.

Another embodiment of this invention is directed to a compound of formula D1, or a pharmaceutically acceptable salt, ester or solvate thereof.

Another embodiment of this invention is directed to a compound of formula A9a1.

Another embodiment of this invention is directed to a compound of formula A9b1.

Another embodiment of this invention is directed to a compound of formula A9c1.

Another embodiment of this invention is directed to a compound of formula A9d1.

Another embodiment of this invention is directed to a compound of formula A9e1.

Another embodiment of this invention is directed to a compound of formula A9f 1.

Another embodiment of this invention is directed to a compound of formula A9g1.

Another embodiment of this invention is directed to a compound of formula A9h1.

Another embodiment of this invention is directed to a compound of formula A9i1.

Another embodiment of this invention is directed to a compound of formula A9j1.

Another embodiment of this invention is directed to a compound of formula A9k1.

Another embodiment of this invention is directed to a compound of formula A9n1.

Another embodiment of this invention is directed to a compound of formula A9o1.

Another embodiment of this invention is directed to a compound of formula A9p1.

Another embodiment of this invention is directed to a compound of formula A9q1.

Another embodiment of this invention is directed to a compound of formula A9a.

Another embodiment of this invention is directed to a compound of formula A9b.

Another embodiment of this invention is directed to a compound of formula A9c.

Another embodiment of this invention is directed to a compound of formula A9d.

Another embodiment of this invention is directed to a compound of formula A9e.

Another embodiment of this invention is directed to a compound of formula A9f.

Another embodiment of this invention is directed to a compound of formula A9g.

Another embodiment of this invention is directed to a compound of formula A9h.

Another embodiment of this invention is directed to a compound of formula A91.

Another embodiment of this invention is directed to a compound of formula A9j.

Another embodiment of this invention is directed to a compound of formula A9k.

Another embodiment of this invention is directed to a compound of formula A91.

Another embodiment of this invention is directed to a compound of formula A9m.

Another embodiment of this invention is directed to a compound of formula A9n.

Another embodiment of this invention is directed to a compound of formula A9o.

Another embodiment of this invention is directed to a compound of formula A9p.

Another embodiment of this invention is directed to a compound of formula A9q.

Another embodiment of this invention is directed to a compound of formula A9r.

Another embodiment of this invention is directed to a compound of formula A9s.

Another embodiment of this invention is directed to a compound of formula A9t.

Another embodiment of this invention is directed to a compound of formula A9u.

Another embodiment of this invention is directed to a compound of formula A9ab.

Another embodiment of this invention is directed to a compound of formula B1.

Another embodiment of this invention is directed to a compound of formula B2.

Another embodiment of this invention is directed to a compound of formula B3.

Another embodiment of this invention is directed to a compound of formula B4.

Another embodiment of this invention is directed to a compound of formula B5.

Another embodiment of this invention is directed to a compound of formula B6.

Another embodiment of this invention is directed to a compound of formula B7.

Another embodiment of this invention is directed to a compound of formula B8.

Another embodiment of this invention is directed to a compound of formula B9.

Another embodiment of this invention is directed to a compound of formula B10.

Another embodiment of this invention is directed to a compound of formula B11.

Another embodiment of this invention is directed to a compound of formula (+)-B11.

Another embodiment of this invention is directed to a compound of formula (−)-B11.

Another embodiment of this invention is directed to a compound of formula B12.

Another embodiment of this invention is directed to a compound of formula B13.

Another embodiment of this invention is directed to a compound of formula B14.

Another embodiment of this invention is directed to a compound of formula B15.

Another embodiment of this invention is directed to a compound of formula B16.

Another embodiment of this invention is directed to a compound of formula B17.

Another embodiment of this invention is directed to a compound of formula B18.

Another embodiment of this invention is directed to a compound of formula B19.

Another embodiment of this invention is directed to a compound of formula B20.

Another embodiment of this invention is directed to a compound of formula B21.

Another embodiment of this invention is directed to a compound of formula B22.

Another embodiment of this invention is directed to a compound of formula B23.

Another embodiment of this invention is directed to a compound of formula C7a.

Another embodiment of this invention is directed to a compound of formula C7b.

Another embodiment of this invention is directed to a compound of formula C7c.

Another embodiment of this invention is directed to a compound of formula C7d.

Another embodiment of this invention is directed to a compound of formula C7e.

Another embodiment of this invention is directed to a compound of formula C7f.

Another embodiment of this invention is directed to a compound of formula D1.

In another embodiment, this invention provides a pharmaceutical composition comprising:

(a) a therapeutically effective amount of at least one compound of Formula (I), or a pharmaceutically acceptable salt, solvate, ester or prodrug thereof, and at least one pharmaceutically acceptable carrier; or

(b) a therapeutically effective amount of at least one compound of Formula (I), or a pharmaceutically acceptable salt, solvate, ester or prodrug thereof, and at least one pharmaceutically acceptable carrier, and a therapeutically effective amount of one or more compounds selected from the group consisting of cholinesterase inhibitors, Aβ antibody inhibitors, gamma secretase inhibitors and beta secretase inhibitors.

In another embodiment, this invention provides a method of treating a central nervous system disorder comprising:

(a) administering a therapeutically effective amount of at least one compound of Formula (I) to a patient in need of such treatment; or

(a) administering a therapeutically effective amount of a pharmaceutical composition comprising a therapeutically effective amount of at least one compound of Formula (I), or a pharmaceutically acceptable salt, solvate, ester or prodrug thereof, and at least one pharmaceutically acceptable carrier; or

(b) administering a therapeutically effective amount of a pharmaceutical composition comprising a therapeutically effective amount of at least one compound of Formula (I), or a pharmaceutically acceptable salt, solvate, ester or prodrug thereof, and at least one pharmaceutically acceptable carrier, and a therapeutically effective amount of one or more compounds selected from the group consisting of cholinesterase inhibitors, Aβ antibody inhibitors, gamma secretase inhibitors and beta secretase inhibitors.

In another embodiment, this invention provides a method of treating Alzheimers disease comprising:

(a) administering a therapeutically effective amount of at least one compound of Formula (I) to a patient in need of such treatment; or

(b) administering a therapeutically effective amount of at least one compound of Formula (I), in combination with a therapeutically effective amount of a BACE inhibitor, to a patient in need of such treatment.

In another embodiment, this invention provides a method of treating Downs syndrome comprising administering a therapeutically effective amount of at least one compound of Formula I to a patient in need of such treatment.

In another embodiment, this invention provides a method of (a) modulating gamma secretase activity comprising administering an effective amount of at least one compound of Formula (I) to a patient in need of such treatment; or

(b) inhibiting the deposition of beta amyloid protein comprising administering an effective amount of at least one compound of Formula (I) to a patient in need of such treatment; or

(c) treating one or more neurodegenerative disease comprising administering an effective amount of at least one compound of Formula (I) to a patient in need of such treatment.

In another embodiment, this invention also provides a method for modulating (including inhibiting, antagonizing and the like) gamma-secretase, comprising administering an effective (i.e., therapeutically effective) amount of one or more compounds of formula (I) to a patient in need of treatment.

In another embodiment, this invention also provides a method for modulating (including inhibiting, antagonizing and the like) gamma-secretase, comprising administering an effective (i.e., therapeutically effective) amount of a compound of formula (I) to a patient in need of treatment.

In another embodiment, this invention also provides a method of treating one or more neurodegenerative diseases, comprising administering an effective (i.e., therapeutically effective) amount of one or more compounds of formula (I) to a patient in need of treatment.

In another embodiment, this invention also provides a method of treating one or more neurodegenerative diseases, comprising administering an effective (i.e., therapeutically effective) amount of a compound of formula (I) to a patient in need of treatment.

In another embodiment, this invention also provides a method of inhibiting the deposition of amyloid protein (e.g., amyloid beta protein) in, on or around neurological tissue (e.g., the brain), comprising administering an effective (i.e., therapeutically effective) amount of one or more compounds of formula (I) to a patient in need of treatment.

In another embodiment, this invention also provides a method of inhibiting the deposition of amyloid protein (e.g., amyloid beta protein) in, on or around neurological tissue (e.g., the brain), comprising administering an effective (i.e., therapeutically effective) amount of a compound of formula (I) to a patient in need of treatment.

In another embodiment, this invention also provides a method of treating Alzheimer's disease, comprising administering an effective (i.e., therapeutically effective) amount of one or more compounds of formula (I) to a patient in need of treatment.

In another embodiment, this invention also provides a method of treating Alzheimer's disease, comprising administering an effective (i.e., therapeutically effective) amount of a compound of formula (I) to a patient in need of treatment.

In another embodiment, this invention also provides a method of treating Alzheimer's disease, comprising administering an effective (i.e., therapeutically effective) amount of one or more compounds of formula (I), in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

In another embodiment, this invention also provides a method of treating Alzheimer's disease, comprising administering an effective (i.e., therapeutically effective) amount of one or more compounds of formula (I), in combination with an effective (i.e., therapeutically effective) amount of one or more compounds selected from the group consisting of Aβ antibody inhibitors, gamma secretase inhibitors and beta secretase inhibitors.

In another embodiment, this invention also provides combinations comprising an effective (i.e., therapeutically effective) amount of one or more compounds of formula (I), in combination with an effective (i.e., therapeutically effective) amount of one or more compounds selected from the group consisting of cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), Aβ antibody inhibitors, gamma secretase inhibitors and beta secretase inhibitors.

In another embodiment, this invention also provides a method of treating Alzheimer's disease, comprising administering an effective (i.e., therapeutically effective) amount of a compound of formula (I), in combination with an effective (i.e., therapeutically effective) amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

In another embodiment, this invention also provides a method of treating Downs syndrome, comprising administering an effective (i.e., therapeutically effective) amount of a compound of formula (I) to a patient in need of treatment.

In another embodiment, this invention also provides a method of treating Downs syndrome, comprising administering an effective (i.e., therapeutically effective) amount of one or more compounds of formula (I), in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H inden-1-one hydrochloride, i.e., donepezil hydrochloride, available as the Aricept® brand of donepezil hydrochloride), to a patient in need of treatment.

In another embodiment, this invention also provides a method of treating Downs syndrome, comprising administering an effective (i.e., therapeutically effective) amount of a compound of formula (I), in combination with an effective (i.e., therapeutically effective) amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a pharmaceutically acceptable salt of a compound of formula (I).

Another embodiment of this invention is directed to a pharmaceutically acceptable ester of a compound of formula (I).

Another embodiment of this invention is directed to a solvate of a compound of formula (I).

Another embodiment of this invention is directed to a compound of formula (I) in isolated form.

Another embodiment of this invention is directed to a compound of formula (I) in pure form.

Another embodiment of this invention is directed to a compound of formula (I) selected from the group consisting of the compounds of formulas IA to IM.

Another embodiment of this invention is directed to a compound of formula (I) selected from the group consisting of the compounds of formulas A9a1 to A9k1, A9n1 to Aq1, A9a to A9u, A9ab, B1-B11, (+)-B11, (−)-B11, B12-B23, C7a to C7f, and D1.

Another embodiment of this invention is directed to a pharmaceutically acceptable salt of a compound selected from the group consisting of the compounds of formulas IA to IM.

Another embodiment of this invention is directed to a pharmaceutically acceptable ester of a compound selected from the group consisting of the compounds of formulas IA to IM.

Another embodiment of this invention is directed to a solvate of a compound selected from the group consisting of the compounds of formulas IA to IM.

Another embodiment of this invention is directed to a pharmaceutically acceptable salt of a compound selected from the group consisting of the compounds of formulas A9a1 to A9k1, A9n1 to Aq1, A9a to A9u, A9ab, B1-B11, (+)-B11, (−)-B11, B12-B23, C7a to C7f, and D1.

Another embodiment of this invention is directed to a pharmaceutically acceptable ester of a compound selected from the group consisting of the compounds of formulas A9a1 to A9k1, A9n1 to Aq1, A9a to A9u, A9ab, B1-B11, (+)-B11, (−)-B11, B12-B23, C7a to C7f, and D1.

Another embodiment of this invention is directed to a solvate of a compound selected from the group consisting of the compounds of formulas A9a1 to A9k1, A9n1 to Aq1, A9a to A9u, A9ab, B1-B11, (+)-B11, (−)-B11, B12-B23, C7a to C7f, and D1.

Another embodiment of this invention is directed to a compound selected from the group consisting of the compounds of formulas A9a1 to A9k1, A9n1 to Aq1, A9a to A9u, A9ab, B1-B11, (+)-B11, (−)-B11, B12-B23, C7a to C7f, and D1 in pure and isolated form.

Another embodiment of this invention is directed to a compound selected from the group consisting of the compounds of formulas A9a1 to A9k1, A9n1 to Aq1, A9a to A9u, A9ab, B1-B11, (+)-B11, (−)-B11, B12-B23, C7a to C7f, and D1 in pure form.

Another embodiment of this invention is directed to a compound selected from the group consisting of the compounds of formulas A9a1 to A9k1, A9n1 to Aq1, A9a to A9u, A9ab, B1-B11, (+)-B11, (−)-B11, B12-B23, C7a to C7f, and D1 in isolated form.

Another embodiment is directed to a pharmaceutical composition comprising an effective amount of one or more (e.g., one) compounds of formula (I) and a pharmaceutically acceptable carrier. And in one example, the compounds of formula (I) are selected from the group consisting of the compounds of formulas A9a1 to A9c1, A9e1 to A9k1, A9n1 to Aq1, A9a to A9u, A9ab, B1-B11, (+)-B11, (−)-B11, B12-B23, C7a to C7f, and D1.

Another embodiment is directed to a pharmaceutical composition comprising an effective amount of a pharmaceutically acceptable salt of one or more (e.g., one) compounds of formula (I) and a pharmaceutically acceptable carrier. And in one example, the pharmaceutically acceptable salt is of a compound selected from the group consisting of the compounds of formulas A9a1 to A9k1, A9n1 to Aq1, A9a to A9u, A9ab, B1-B11, (+)-B11, (−)-B11, B12-B23, C7a to C7f, and D1.

Another embodiment is directed to a pharmaceutical composition comprising an effective amount of a pharmaceutically acceptable ester of one or more (e.g., one) compounds of formula (I) and a pharmaceutically acceptable carrier. And in one example, the pharmaceutically acceptable ester is of a compound selected from the group consisting of the compounds of formulas A9a1 to A9k1, A9n1 to Aq1, A9a to A9u, A9ab, B1-B11, (+)-B11, (−)-B11, B12-B23, C7a to C7f, and D1.

Another embodiment is directed to a pharmaceutical composition comprising an effective amount of a solvate of one or more (e.g., one) compounds of formula (I) and a pharmaceutically acceptable carrier. And in one example, the solvate is of a compound selected from the group consisting of the compounds of formulas A9a1 to A9k1, A9n1 to Aq1, A9a to A9u, A9ab, B1-B11, (+)-B11, (−)-B11, B12-B23, C7a to C7f, and D1.

Another embodiment of this invention is directed to a pharmaceutical composition comprising an effective amount of one or more (e.g., one) compounds of formula (I), and an effective amount of one or more (e.g., one) other pharmaceutically active ingredients (e.g., drugs) (as described below, for example), and a pharmaceutically acceptable carrier. Examples of the other pharmaceutically active ingredients include, but are not limited to drugs selected form the group consisting of: (a) drugs useful for the treatment of Alzheimer's disease, (b) drugs useful for inhibiting the deposition of amyloid protein (e.g., amyloid beta protein) in, on or around neurological tissue (e.g., the brain), (c) drugs useful for treating neurodegenerative diseases, and (d) drugs useful for inhibiting gamma-secretase. And in one example, the compounds of formula (I) are selected from the group consisting of the compounds of formulas A9a1 to A9k1, A9n1 to Aq1, A9a to A9u, A9ab, B1-B11, (+)-B11, (−)-B11, B12-B23, C7a to C7f, and D1.

Another embodiment of this invention is directed to a pharmaceutical composition comprising an effective amount of one or more (e.g., one) compounds of formula (I), and effective amount of one or more BACE inhibitors, and a pharmaceutically acceptable carrier. And in one example, the compounds of formula (I) are selected from the group consisting of the compounds of formulas A9a1 to A9k1, A9n1 to Aq1, A9a to A9u, A9ab, B1-B11, (+)-B11, (−)-B11, B12-B23, C7a to C7f, and D1.

Another embodiment of this invention is directed to a pharmaceutical composition comprising an effective amount of one or more (e.g., one) compounds of formula (I), and effective amount of one or more cholinesterase inhibitors (e.g., acetyl- and/or butyrylchlolinesterase inhibitors), and a pharmaceutically acceptable carrier. And in one example, the compounds of formula (I) are selected from the group consisting of the compounds of formulas A9a1 to A9k1, A9n1 to Aq1, A9a to A9u, A9ab, B1-B11, (+)-B11, (−)-B11, B12-B23, C7a to C7f, and D1.

Another embodiment of this invention is directed to a pharmaceutical composition comprising an effective amount of one or more (e.g., one) compounds of formula (I), and effective amount of one or more muscarinic antagonists (e.g., m₁ or m₂ antagonists), and a pharmaceutically acceptable carrier. And in one example, the compounds of formula (I) are selected from the group consisting of the compounds of formulas A9a1 to A9k1, A9n1 to Aq1, A9a to A9u, A9ab, B1-B11, (+)-B11, (−)-B11, B12-B23, C7a to C7f, and D1.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of one or more (e.g. one) compounds of formula (I) and the administration of one or more (e.g., one) other pharmaceutical active ingredients (e.g., drugs). The compounds of formula (I) and the other drugs can be administered separately (i.e., each is in its own separate dosage form), or the compounds of formula (I) can be combined with the other drugs in the same dosage form. And in one example, the compounds of formula (I) are selected from the group consisting of the compounds of formulas A9a1 to A9k1, A9n1 to Aq1, A9a to A9u, A9ab, B1-B11, (+)-B11, (−)-B11, B12-B23, C7a to C7f, and D1.

Thus, other embodiments of this invention are directed to any one of the methods of treatment, or methods of inhibiting, described herein, wherein the compound of formula (I) is used in combination with an effective amount of one or more other pharmaceutically active ingredients selected from the group consisting of: BACE inhibitors (beta secretase inhibitors), muscarinic antagonists (e.g., m₁ or m₂ antagonists), cholinesterase inhibitors (e.g., acetyl- and/or butyrylchlolinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors and cholesterol absorption inhibitors (e.g., ezetimibe). And in one example, the compounds of formula (I) are selected from the group consisting of the compounds of formulas A9a1 to A9k1, A9n1 to Aq1, A9a to A9u, A9ab, 61-B11, (+)-B11, (−)-B11, B12-B23, C7a to C7f, and D1.

Thus, another embodiment of this invention is directed to a method of treating Alzheimer's disease comprising administering one or more (e.g., one) compounds of formula (I) in combination with an effective amount of one or more other pharmaceutically active ingredients selected from the group consisting of: BACE inhibitors (beta secretase inhibitors), muscarinic antagonists (e.g., m₁ or m₂ antagonists), cholinesterase inhibitors (e.g., acetyl- and/or butyrylchiolinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors and cholesterol absorption inhibitors (e.g., ezetimibe), to a patient in need of such treatment. And in one example, the compounds of formula (I) are selected from the group consisting of the compounds of formulas A9a1 to A9k1, A9n1 to Aq1, A9a to A9u, A9ab, B1-B11, (+)-B11, (−)-B11, B12-B23, C7a to C7f, and D1.

In another embodiment, this invention provides a method of treating Alzheimer's disease, comprising administering an effective (i.e., therapeutically effective) amount of one or more (e.g., one) compounds of formula (I), in combination with an effective (i.e., therapeutically effective) amount of one or more BACE inhibitors. And in one example, the compounds of formula (I) are selected from the group consisting of the compounds of formulas A9a1 to A9k1, A9n1 to Aq1, A9a to A9u, A9ab, B1-B11, (+)-B11, (−)-B11, B12-B23, C7a to C7f, and D1.

In another embodiment, this invention provides a method of treating mild cognitive impairment, comprising administering an effective amount of one or more (e.g., one) compounds of formula (I) to a patient in need of treatment. And in one example, the compounds of formula (I) are selected from the group consisting of the compounds of formulas A9a1 to A9k1, A9n1 to Aq1, A9a to A9u, A9ab, B1-B11, (+)-B11, (−)-B11, B12-B23, C7a to C7f, and D1.

In another embodiment, this invention provides a method of treating glaucoma, comprising administering an effective amount of one or more (e.g., one) compounds of formula (I) to a patient in need of treatment. And in one example, the compounds of formula (I) are selected from the group consisting of the compounds of formulas A9a1 to A9k1, A9n1 to Aq1, A9a to A9u, A9ab, B1-B11, (+)-B11, (−)-B11, B12-B23, C7a to C7f, and D1.

In another embodiment, this invention provides a method of treating cerebral amyloid angiopathy, comprising administering an effective amount of one or more (e.g., one) compounds of formula (I) to a patient in need of treatment. And in one example, the compounds of formula (I) are selected from the group consisting of the compounds of formulas A9a1 to A9k1, A9n1 to Aq1, A9a to A9u, A9ab, B1-B11, (+)-B11, (−)-B11, B12-B23, C7a to C7f, and D1.

In another embodiment, this invention provides a method of treating stroke, comprising administering an effective amount of one or more (e.g., one) compounds of formula (I) to a patient in need of treatment. And in one example, the compounds of formula (I) are selected from the group consisting of the compounds of formulas A9a1 to A9k1, A9n1 to Aq1, A9a to A9u, A9ab, B1-B11, (+)-B11, (−)-B11, B12-B23, C7a to C7f, and D1.

In another embodiment, this invention provides a method of treating dementia, comprising administering an effective amount of one or more (e.g., one) compounds of formula (I) to a patient in need of treatment. And in one example, the compounds of formula (I) are selected from the group consisting of the compounds of formulas A9a1 to A9k1, A9n1 to Aq1, A9a to A9u, A9ab, B1-B11, (+)-B11, (−)-B11, B12-B23, C7a to C7f, and D1.

In another embodiment, this invention provides a method of treating microgliosis, comprising administering an effective amount of one or more (e.g., one) compounds of formula (I) to a patient in need of treatment. And in one example, the compounds of formula (I) are selected from the group consisting of the compounds of formulas A9a1 to A9k1, A9n1 to Aq1, A9a to A9u, A9ab, B1-B11, (+)-B11, (−)-B11, B12-B23, C7a to C7f, and D1.

In another embodiment, this invention provides a method of treating brain inflammation, comprising administering an effective amount of one or more (e.g., one) compounds of formula (I) to a patient in need of treatment. And in one example, the compounds of formula (I) are selected from the group consisting of the compounds of formulas A9a1 to A9k1, A9n1 to Aq1, A9a to A9u, A9ab, 61-B11, (+)-B11, (−)-B11, B12-B23, C7a to C7f, and D1.

In another embodiment, this invention provides a method of treating olfactory function loss, comprising administering an effective amount of one or more (e.g., one) compounds of formula (I) to a patient in need of treatment. And in one example, the compounds of formula (I) are selected from the group consisting of the compounds of formulas A9a1 to A9k1, A9n1 to Aq1, A9a to A9u, A9ab, B1-B11, (+)-B11, (−)-B11, B12-B23, C7a to C7f, and D1.

In another embodiment this invention also provides pharmaceutical compositions comprising a combination of an effective amount of one or more (e.g., one) compounds of formula (I), in combination with an effective amount of one or more compounds selected from the group consisting of cholinesterase inhibitors, Aβ antibody inhibitors, gamma secretase inhibitors and beta secretase inhibitors. The pharmaceutical compositions also comprise a pharmaceutically acceptable carrier. And in one example, the compounds of formula (I) are selected from the group consisting of the compounds of formulas A9a1 to A9k1, A9n1 to Aq1, A9a to A9u, A9ab, B1-B11, (+)-B11, (−)-B11, B12-B23, C7a to C7f, and D1.

In another embodiment this invention also provides combinations (i.e., pharmaceutical compositions) comprising an effective (i.e., therapeutically effective) amount of one or more (e.g., one) compounds of formula (I), in combination with an effective (i.e., therapeutically effective) amount of one or more compounds selected from the group consisting of cholinesterase inhibitors (such as, for example, (4-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), A13 antibody inhibitors, gamma secretase inhibitors and beta secretase inhibitors. The pharmaceutical compositions also comprise a pharmaceutically acceptable carrier. And in one example, the compounds of formula (I) are selected from the group consisting of the compounds of formulas A9a1 to A9k1, A9n1 to Aq1, A9a to A9u, A9ab, B1-B11, (+)-B11, (−)-B11, B12-B23, C7a to C7f, and D1.

This invention also provides a kit comprising, in separate containers, in a single package, pharmaceutical compositions for use in combination, wherein one container comprises an effective amount of a compound of formula (I) in a pharmaceutically acceptable carrier, and another container (i.e., a second container) comprises an effective amount of another pharmaceutically active ingredient (as described above), the combined quantities of the compound of formula (I) and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase. And in one example, the compounds of formula (I) are selected from the group consisting of the compounds of formulas A9a1 to A9k1, A9n1 to Aq1, A9a to A9u, A9ab, B1-B11, (+)-B11, (−)-B11, B12-B23, C7a to C7f, and D1.

Compounds of formula (I) include compounds of formulas: IA to IM, A9a1 to A9k1, A9n1 to Aq1, A9a to A9u, A9ab, B1-B11, (+)-B11, (−)-B11, B12-B23, C7a to C7f, and D1.

Thus, a compound of the formula IA to IM can be used instead of a compound of formula (I) in any one of the embodiments directed to the compounds of formula (I).

Also, a compound of the formula A9a1 to A9k1, A9n1 to Aq1, A9a to A9u, A9ab, B1-B11, (+)-B11, (−)-B11, B12-B23, C7a to C7f, or D1 can be used instead of a compound of formula (I) in any one of the embodiments directed to the compounds of formula (I).

Examples of cholinesterase inhibitors are tacrine, donepezil, rivastigmine, galantamine, pyridostigmine and neostigmine, with tacrine, donepezil, rivastigmine and galantamine being preferred.

Examples of m₁ antagonists are known in the art. Examples of m₂ antagonists are also known in the art; in particular, m₂ antagonists are disclosed in U.S. Pat. Nos. 5,883,096; 6,037,352; 5,889,006; 6,043,255; 5,952,349; 5,935,958; 6,066,636; 5,977,138; 6,294,554; 6,043,255; and 6,458,812; and in WO 03/031412, all of which are incorporated herein by reference.

Examples of BACE inhibitors include those described in: US2005/0119227 published Jun. 2, 2005 (see also WO2005/016876 published Feb. 24, 2005), US2005/0043290 published Feb. 24, 2005 (see also WO2005/014540 published Feb. 17, 2005), WO2005/058311 published Jun. 30, 2005 (see also US2007/0072852 published Mar. 29, 2007), US2006/0111370 published May 25, 2006 (see also WO2006/065277 published Jun. 22, 2006), U.S. application Ser. No. 11/710,582 filed Feb. 23, 2007, US2006/0040994 published Feb. 23, 2006 (see also WO2006/014762 published Feb. 9, 2006), WO2006/014944 published Feb. 9, 2006 (see also US2006/0040948 published Feb. 23, 2006), WO2006/138266 published Dec. 28, 2006 (see also US2007/0010667 published Jan. 11, 2007), WO2006/138265 published Dec. 28, 2006, WO2006/138230 published Dec. 28, 2006, WO2006/138195 published Dec. 28, 2006 (see also US2006/0281729 published Dec. 14, 2006), WO2006/138264 published Dec. 28, 2006 (see also US2007/0060575 published Mar. 15, 2007), WO2006/138192 published Dec. 28, 2006 (see also US2006/0281730 published Dec. 14, 2006), WO2006/138217 published Dec. 28, 2006 (see also US2006/0287294 published Dec. 21, 2006), US2007/0099898 published May 3, 200 (see also WO2007/050721 published May 3, 2007), WO2007/053506 published May 10, 2007 (see also US2007/099875 published May 3, 2007), U.S. application Ser. No. 11/759,336 filed Jun. 7, 2007, U.S. Application Ser. No. 60/874,362 filed Dec. 12, 2006, and U.S. Application Ser. No. 60/874,419 filed Dec. 12, 2006, the disclosures of each being incorporated herein by reference thereto.

As used above, and throughout this disclosure, the following terms, unless otherwise indicated, shall be understood to have the following meanings:

“Effective Amount” means a therapeutically effective amount.

“One or more” means there is one or more than one (e.g., 1-3, or 1-2, or 1).

“At least one” means there is at least one or more than one (e.g., 1-3, or 1-2, or 1).

“Patient” includes both human and animals.

“Mammal” means humans and other mammalian animals.

It is noted that the carbons of formula (I) and other formulas herein may be replaced with 1 to 3 silicon atoms so long as all valency requirements are satisfied.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. “Alkyl” may be unsubstituted or optionally substituted by one or more substituents which may be the same or different, each substituent being independently selected from the group consisting of halo, alkyl, aryl, cycloalkyl, cyano, hydroxy, alkoxy, alkylthio, amino, oxime (e.g., ═N—OH), —NH(alkyl), —NH(cycloalkyl), —N(alkyl)₂, —O—C(O)-alkyl, —O—C(O)-aryl, —O—C(O)-cycloalkyl, carboxy and —C(O)O-alkyl. Non-limiting examples of suitable alkyl groups include methyl, ethyl, n-propyl, isopropyl and t-butyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. “Alkenyl” may be unsubstituted or optionally substituted by one or more substituents which may be the same or different, each substituent being independently selected from the group consisting of halo, alkyl, aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkylene” means a difunctional group obtained by removal of a hydrogen atom from an alkyl group that is defined above. Non-limiting examples of alkylene include methylene, ethylene and propylene.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. “Alkynyl” may be unsubstituted or optionally substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. “Heteroaryl” may also include a heteroaryl as defined above fused to an aryl as defined above. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl- group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl- group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Cycloalkylalkyl” means a cycloalkyl moiety as defined above linked via an alkyl moiety (defined above) to a parent core. Non-limiting examples of suitable cycloalkylalkyls include cyclohexylmethyl, adamantylmethyl and the like.

“Cycloalkenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl.

“Cycloalkenylalkyl” means a cycloalkenyl moiety as defined above linked via an alkyl moiety (defined above) to a parent core. Non-limiting examples of suitable cycloalkenylalkyls include cyclopentenylmethyl, cyclohexenylmethyl and the like.

“Halogen” means fluorine, chlorine, bromine, or iodine. Preferred are fluorine, chlorine and bromine. “Halo” refers to fluoro, chloro, bromo or iodo.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, —O—C(O)-alkyl, —O—C(O)-aryl, —O—C(O)-cycloalkyl, —C(═N—CN)—NH₂, —C(═NH)—NH₂, —C(═NH)—NH(alkyl), oxime (e.g., ═N—OH), Y₁Y₂N—, Y₁Y₂N-alkyl-, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moiety are methylene dioxy, ethylenedioxy, —C(CH₃)₂— and the like which form moieties such as, for example:

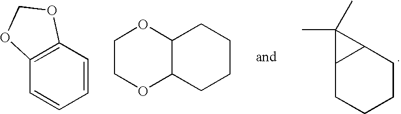

“Heteroarylalkyl” means a heteroaryl moiety as defined above linked via an alkyl moiety (defined above) to a parent core. Non-limiting examples of suitable heteroaryls include 2-pyridinylmethyl, quinolinylmethyl and the like.

“Heterocyclyl” or “heterocycloalkyl” means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like.

Heterocyclyl also includes rings wherein ═O replaces two available hydrogens on the same carbon atom (i.e., heterocyclyl includes rings having a carbonyl group in the ring). An example of such a heterocyclyl ring is pyrrolidone:

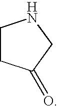

“Heterocyclylalkyl” or “heterocycloalkylalkyl” means a heterocyclyl moiety as defined above linked via an alkyl moiety (defined above) to a parent core. Non-limiting examples of suitable heterocyclylalkyls include piperidinylmethyl, piperazinylmethyl and the like.

“Heterocyclenyl” means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclenyl” also includes rings wherein ═O replaces two available hydrogens on the same carbon atom (i.e., heterocyclenyl includes rings having a carbonyl group in the ring). An example of such a heterocyclenyl ring is pyrrolidinone:

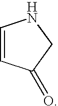

“Heterocyclenylalkyl” means a heterocyclenyl moiety as defined above linked via an alkyl moiety (defined above) to a parent core.

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring:

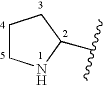

there is no —OH attached directly to carbons marked 2 and 5.

It should also be noted that tautomeric forms such as, for example, the moieties:

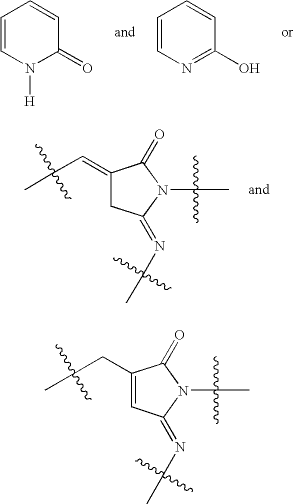

are considered equivalent in certain embodiments of this invention.

“Alkynylalkyl” means an alkynyl-alkyl- group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylmethyl.

“Heteroaralkyl” means a heteroaryl-alkyl- group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl- group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1- naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound' or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “purified”, “in purified form” or “in isolated and purified form” for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or in Formula (I), its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to yield a compound of Formula (I) or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of Formula (I) or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a compound of Formula (I) contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)_{alkyl})₂ or glycosyl (the radical resulting from the removal of a hydroxyl group of the hemiacetal form of a carbohydrate), and the like.

If a compound of Formula (I) incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N- or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

The compounds of Formula (I) can form salts which are also within the scope of this invention. Reference to a compound of Formula (I) herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula (I) contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula (I) may be formed, for example, by reacting a compound of Formula (I) with an amount of acid or base, such as an equivalent amount, in a medium such as one in which the salt precipitates or in an aqueous medium followed by lyophilization. Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

Compounds of Formula (I), and salts, solvates, esters and prodrugs thereof, may exist in their tautomeric form (for example, as an amide, enol, keto or imino ether). All such tautomeric forms are contemplated herein as part of the present invention.

The compounds of Formula (I) may contain asymmetric or chiral centers, and, therefore, exist in different stereoisomeric forms. It is intended that all stereoisomeric forms of the compounds of Formula (I) as well as mixtures thereof, including racemic mixtures, form part of the present invention. In addition, the present invention embraces all geometric and positional isomers. For example, if a compound of Formula (I) incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this invention. Enantiomers can also be separated by use of chiral HPLC column.

It is also possible that the compounds of Formula (I) may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of Formula (I) incorporates a double bond or a fused ring, both the cis- and trans-forms, as well as mixtures, are embraced within the scope of the invention. Also, for example, all keto-enol and imine-enamine forms of the compounds are included in the invention.) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵ N, ¹⁸O, ¹⁷O, ³¹P, ³²P, ³⁵S, ¹⁸F, and ³⁶Cl, respectly.

Certain isotopically-labelled compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of Formula (I) can generally be prepared by following procedures analogous to those disclosed in the Schemes and/or in the Examples hereinbelow, by substituting an appropriate isotopically labelled reagent for a non-isotopically labelled reagent.

Polymorphic forms of the compounds of Formula (I), and of the salts, solvates, esters and prodrugs of the compounds of Formula (I), are intended to be included in the present invention.

The compounds according to the invention can have pharmacological properties; in particular, the compounds of Formula (I) can be modulators of gamma secretase (including inhibitors, antagonists and the like).

More specifically, the compounds of Formula (I) can be useful in the treatment of a variety of disorders of the central nervous system including, for example, including, but not limited to, Alzheimer's disease, AIDS-related dementia, Parkinson's disease, amyotrophic lateral sclerosis, retinitis pigmentosa, spinal muscular atrophy and cerebellar degeneration and the like.

Another aspect of this invention is a method of treating a mammal (e.g., human) having a disease or condition of the central nervous system by administering a therapeutically effective amount of at least one compound of Formula (I), or a pharmaceutically acceptable salt, solvate, ester or prodrug of said compound to the mammal.

A preferred dosage is about 0.001 to 500 mg/kg of body weight/day of the compound of Formula (I). An especially preferred dosage is about 0.01 to 25 mg/kg of body weight/day of a compound of Formula (I), or a pharmaceutically acceptable salt or solvate of said compound.

The compounds of this invention may also be useful in combination (administered together or sequentially) with one or more additional agents listed above.

The compounds of this invention may also be useful in combination (administered together or sequentially) with one or more compounds selected from the group consisting of Aβ antibody inhibitors, gamma secretase inhibitors and beta secretase inhibitors.

If formulated as a fixed dose, such combination products employ the compounds of this invention within the dosage range described herein and the other pharmaceutically active agent or treatment within its dosage range.

Accordingly, in an aspect, this invention includes combinations comprising an amount of at least one compound of Formula (I), or a pharmaceutically acceptable salt, solvate, ester or prodrug thereof, and an amount of one or more additional agents listed above wherein the amounts of the compounds/treatments result in desired therapeutic effect.

The pharmacological properties of the compounds of this invention may be confirmed by a number of pharmacological assays. Certain assays are exemplified later in this document.

This invention is also directed to pharmaceutical compositions which comprise at least one compound of Formula (I), or a pharmaceutically acceptable salt, solvate, ester or prodrug of said compound and at least one pharmaceutically acceptable carrier.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

The compounds of this invention may also be delivered subcutaneously.

Preferably the compound is administered orally.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 1 mg to about 100 mg, preferably from about 1 mg to about 50 mg, more preferably from about 1 mg to about 25 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 200 mg/day, in two to four divided doses.

Another aspect of this invention is a kit comprising a therapeutically effective amount of at least one compound of Formula (I), or a pharmaceutically acceptable salt, solvate, ester or prodrug of said compound and a pharmaceutically acceptable carrier, vehicle or diluent.

Yet another aspect of this invention is a kit comprising an amount of at least one compound of Formula (I), or a pharmaceutically acceptable salt, solvate, ester or prodrug of said compound and an amount of at least one additional agent listed above, wherein the amounts of the two or more ingredients result in desired therapeutic effect.

The invention disclosed herein is exemplified by the following illustrative examples which should not be construed to limit the scope of the disclosure. Alternative mechanistic pathways and analogous structures will be apparent to those skilled in the art.

Where NMR data are presented, ¹H spectra were obtained on either a Varian VXR-200 (200 MHz, ¹H), Varian Gemini-300 (300 MHz) or XL-400 (400 MHz) and are reported as ppm down field from Me₄Si with number of protons, multiplicities, and coupling constants in Hertz indicated parenthetically. Where LC/MS data are presented, analyses was performed using an Applied Biosystems API-100 mass spectrometer and Shimadzu SCL-10A LC column: Altech platinum C18, 3 micron, 33 mm×7 mm ID; gradient flow: 0 min-10% CH₃CN, 5 min-95% CH₃CN, 7 min-95% CH₃CN, 7.5 min-10% CH₃CN, 9 min-stop. The retention time and observed parent ion are given.

### Method A, Step 1

### Preparation of Methyl-2-isothiocyanatoacetate

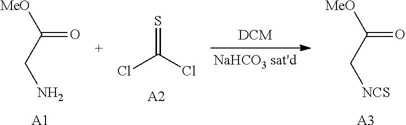

To a round bottom flask held at 0° C. was added the hydrochloride salt of glycine methyl ester A1 (2.00 g, 15.9 mmol) and thiophosgene A2 (2.67 mL, 35.0 mmol) into a solution of dichloromethane (10 mL) and sat'd NaHCO₃ aq solution (10 mL). The reaction was stirred vigorously while warming to room temperature over 16h. The mixture was extracted with dichloromethane and water. The organic portion was dried over sodium sulfate, filtered, and concentrated in vacuo to yield 0.42 g of methyl-2-isothiocyanatoacetate A3.

¹H NMR (CDCl₃) δ (ppm): 3.83 (s, 3H); 4.25 (s, 2H).

### Method A, Step 2

### Preparation of (R)-methyl-2-(3(1-(4-fluorophenyl)ethyl)thiouriedo)acetate A5a

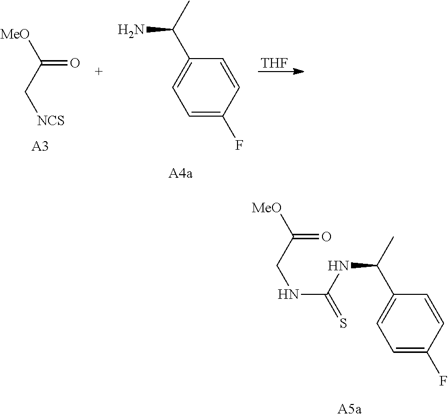

To a round bottom flask was added methyl-2-isothiocyanatoacetate A3 (0.42 g, 3.2 mmol) and (S)-1-(4-fluorophenyl)ethylamine A4a in (0.48 mL, 3.5 mmol) in tetrahydrofuran (5 mL) and stir at room temperature for 1 h. The mixture was extracted with ethyl acetate and water (2×), then 1M HCl aq (2×) then sat'd NaHCO₃ aq solution (2×). The organic portion was dried over sodium sulfate, filtered, and concentrated in vacuo to yield 0.80 g of (R)-methyl-2-(3(1-(4-fluorophenyl)ethyl)thiouriedo)acetate A5a.

¹H NMR (CDCl₃) δ (ppm): 1.49 (d, 3H); 3.71 (s, 3H); 4.24 (d, 1H); 4.36 (d, 1H); 4.91 (br s, 1H); 6.17 (br s, 1H); 6.70 (br s, 1H); 7.01 (t, 2H); 7.28 (m, 2H).

### Method A, Step 3

### Preparation of (R)-3(1-(4-fluorophenyl)ethyl)-2-thioxoimidazolidin-4-one A6a

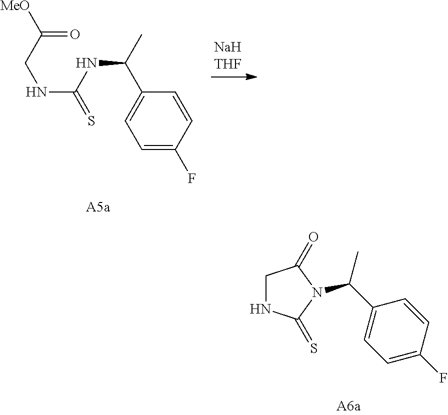

To a round bottom flask held at 0° C. containing a solution of sodium hydride (114 mg, 2.85 mmol) in anhydrous tetrahydrofuran (5 mL), was slowly added a solution of (R)-methyl-2-(3(1-(4-fluorophenyl)ethyl)thiouriedo)acetate A5a (700 mg, 2.0 mmol) in tetrahydrofuran (10 mL) via addition funnel over a period of 45 minutes. The reaction was allowed to warm to room temperature and stirred an additional 30 minutes. The reaction mixture was diluted with ethyl acetate and extracted with 1N HCl aq (2×30 mL), then brine (40 mL). The organic portion was dried over sodium sulfate, filtered, and concentrated in vacuo to yield 450 mg of (R)-3(1-(4-fluorophenyl)ethyl)-2-thioxoimidazolidin-4-one A6a.

¹H NMR (CDCl₃) δ (ppm): 1.85 (d, 3H); 4.95 (q, 2H); 5.99 (q, 1H); 7.00 (t, 2H); 7.51 (m, 2H). ESI MS (M+1)⁺m/z calcd for C₁₁H₁₂FN₂OS⁺=239.1, found m/z=239.1.

### Method A, Step 4

### Preparation of (R)-3(1-(4-fluorophenyl)ethyl)-5-(3-methoxy-4-(4-methyl-1H-imidazol-1-yl)benzylidine)-2-thioxoimidazolidin-4-one A7a

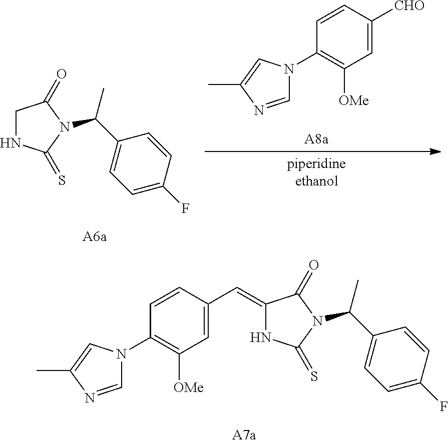

In a round bottom flask piperidine (0.41 mL, 4.2 mmol) was added to a solution of (3-methoxy-4-(4-methyl-1H-imidazol-1-yl)benzaldehyde A8a (429 mg, 2.0 mmol) and (R)-3(1-(4-fluorophenyl)ethyl)-2-thioxoimidazolidin-4-one A6a (450 mg, 1.9 mmol) in ethanol (20 mL). This mixture was stirred at reflux temperature for 16 h. The reaction mixture was cooled to room temperature, diluted with ethyl acetate, and extracted with water then brine. The organic portion was dried over sodium sulfate, filtered, and concentrated in vacuo to yield the crude product. The mixture was chromatographed (40 g silica gel, 0 to 10% MeOH/dichloromethane). This mixture was further purified by diluting the crude product in dichloromethane and allowing it shake with an excess of resin bound PS-TsNHNH₂ at room temperature overnight. The mixture was filtered and concentrated to produce 0.65 g (R)-3(1-(4-fluorophenyl)ethyl)-5-(3-methoxy-4-(4-methyl-1H-imidazol-1-yl)benzylidine)-2-thioxoimidazolidin-4-one A7a.

¹H NMR (CDCl₃) δ (ppm): 1.93 (d, 3H); 2.32 (s, 3H); 3.73 (s, 3H); 6.13 (q, 1H); 6.47 (s, 1H); 6.86 (s, 1H); 6.97-7.06 (m, 51-1); 7.18 (s, 1H); 7.56 (m, 2H). ESI MS (M+1)⁺ m/z calcd for C₂₃H₂₂FN₄O₂S⁺=437.1, found m/z=437.2.

The following compound was prepared in a similar fashion:

| | | | Observed | Retention |
|---|---|---|---|---|
| | | | MS (ESI) | Time |
| Example | Structure | MW | m/z | (min) |
| A7b | 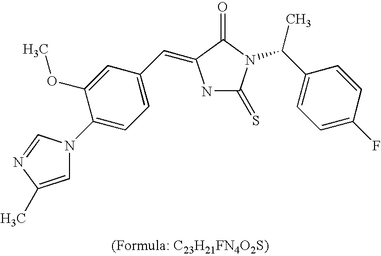 | 436.5 | 437.2 | 3.23 |
| A7c | 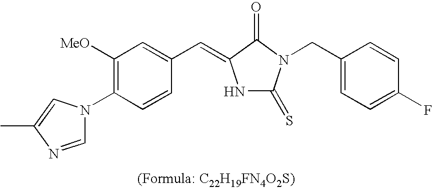 | 422.5 | 423.2 | 2.98 |
| A7d | 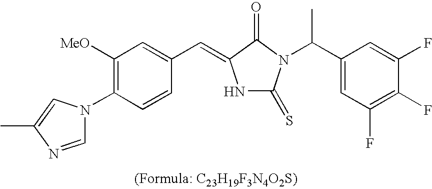 | 472.5 | 473.3 | 3.66 |
| A7e | 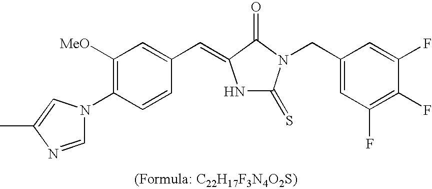 | 458.5 | 459.3 | 3.13 |

### Method A, Step 5

### Preparation of (R)-3(1-(4-fluorophenyl)ethyl)-3-imino-5-(3-methoxy-4-(4-methyl-1H-imidazol-1-yl)benzylidine)-2-imidazolidin-4-one A9a

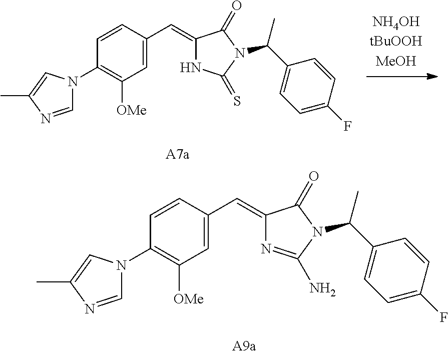

In a sealed vial was added (R)-3(1-(4-fluorophenyl)ethyl)-5-(3-methoxy-4-(4-methyl-1H-imidazol-1-yl)benzylidine)-2-thioxoimidazolidin-4-one Ala (13.5 mg, 0.03 mmol) in MeOH (1 mL), 15N ammonium hydroxide (0.5 mL) and a 70% solution of tert-butyl hydroperoxide in water (0.5 mL). The mixture was agitated at room temperature for 16 h. The mixture was concentrated in vacuo and purified by reverse phase chromatography to produce 3.5 mg of (R)-3(1-(4-fluorophenyl)ethyl)-3-imino-5-(3-methoxy-4-(4-methyl-1H-imidazol-1-yl)benzylidine)-2-imidazolidin-4-one A9a.

¹H NMR (CDCl₃) δ (ppm): 1.89 (d, 3H); 2.27 (s, 3H); 3.93 (s, 3H); 5.39 (q, 1H); 5.49 (s, 1H); 6.53 (s, 1H); 7.10 (t, 2H); 7.17 (br s, 1H); 7.33 (d, 1H); 7.40-7.46 (m, 2H); 7.51 (d, 1H); 8.05 (br s, 1H); 8.08 (s, 1H); 8.30 (br s, 1H). ESI MS (M+1)⁺ m/z calcd for C₂₃H₂₃FN₅O₂⁺=420.2, found m/z=420.2, retention time 2.27 min.

The following compounds were prepared in a similar fashion:

| | | | DATA | Retention |
|---|---|---|---|---|
| | | | MS(ESI) | Time |
| Example | Structure | MW | m/z | (min) |
| A9b | 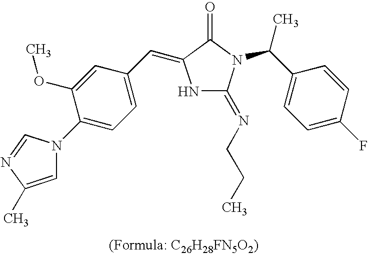 | 461.5 | 462.3 | 2.93 |
| A9c | 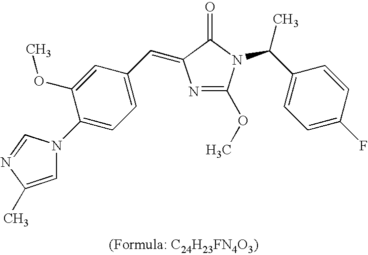 | 434.5 | 435.2 | 3.15 |
| A9d | 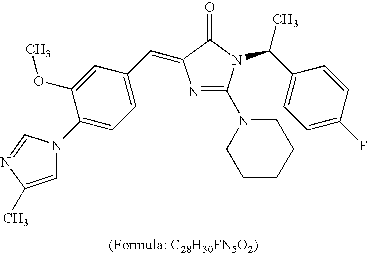 | 487.6 | 488.3 | 3.28 |
| A9e | 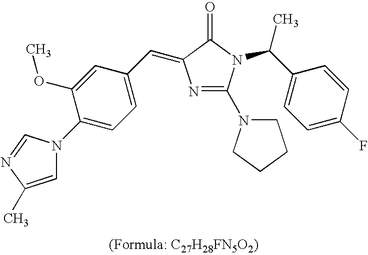 | 473.5 | 474.3 | 2.82 |
| A9f | 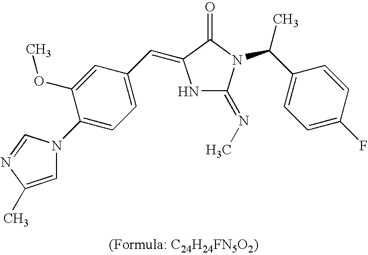 | 433.5 | 434.2 | 2.60 |
| A9g | 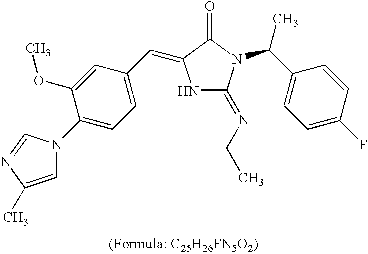 | 447.5 | 448.2 | 2.77 |
| A9h | 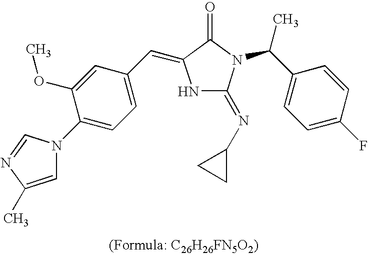 | 459.5 | 460.3 | 2.62 |
| A9i | 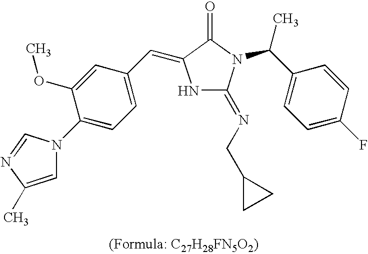 | 473.5 | 474.3 | 3.02 |
| A9j | 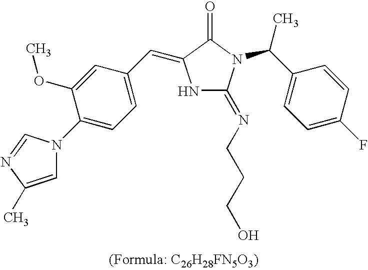 | 477.5 | 478.3 | 2.56 |
| A9k | 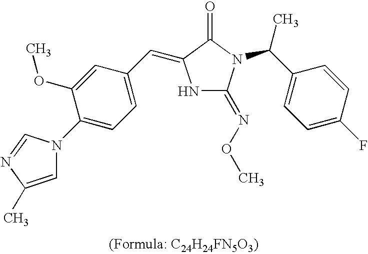 | 449.5 | 450.2 | 3.05 |
| A9l | 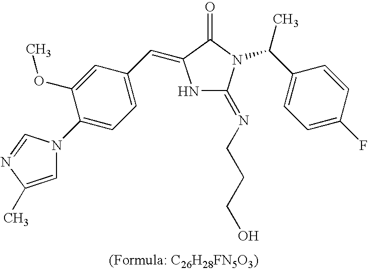 | 477.5 | 478.3 | 2.60 |
| A9m | 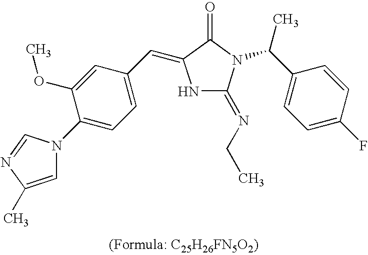 | 447.5 | 448.2 | 2.80 |
| A9n | 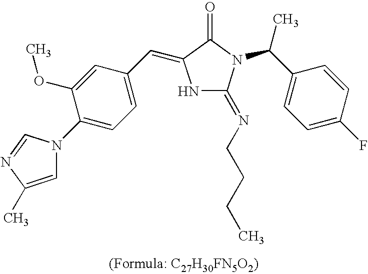 | 475.6 | 476.3 | 3.14 |
| A9o | 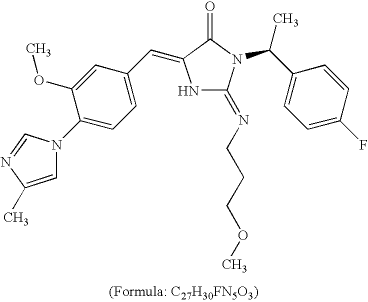 | 491.6 | 492.3 | 2.90 |
| A9p | 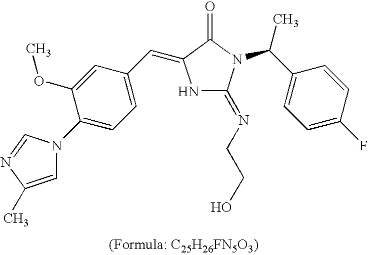 | 463.5 | 464.3 | 2.55 |
| A9q | 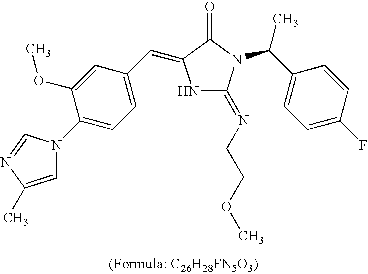 | 477.5 | 478.3 | 2.83 |
| A9r | 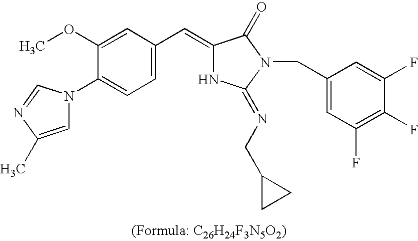 | 495.5 | 496.3 | 3.24 |
| A9s | 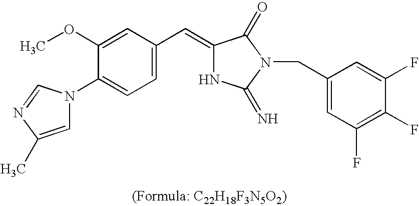 | 441.4 | 442.2 | 2.47 |
| A9t | 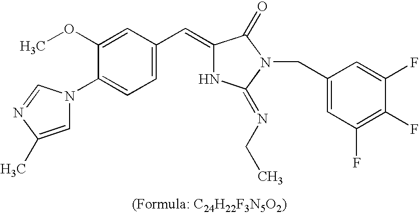 | 469.4 | 470.3 | 3.13 |
| A9u | 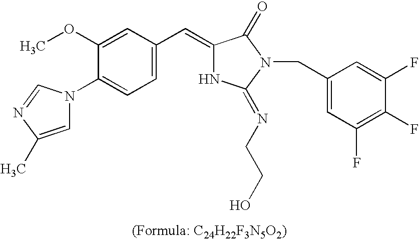 | 485.4 | 486.3 | 2.67 |

### Method B

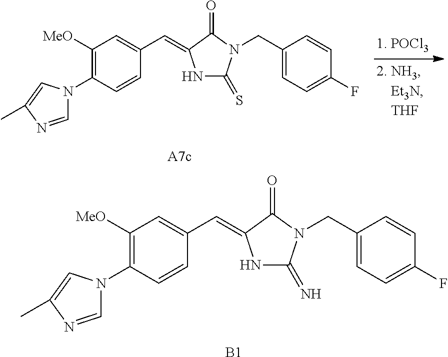

Compound A7c was synthesized from p-fluorobenzyl amine using a method similar to Method A, Steps 2-4.

A mixture of compound A7c (107 mg, 0.253 mmol) and POCl₃ (2.5 mL) was heated in a microwave reactor at 170° C. for 45 minutes. This resulting solution was diluted with CH₂Cl₂, transferred to a round bottom flask, and concentrated under reduced pressure. The residue was dissolved in tetrahydrofuran (2.5 mL), and then triethylamine (0.32 mL) and 7 M NH₃ in methanol (0.72 mL). This mixture was then sealed and stirred for 4 days. The resulting mixture was absorbed onto silica gel and chromatographed (MeOH/aq. NH₄OH/CH₂Cl₂) to afford compound B1 (53 mg, 52%) as a yellow solid. ¹HNMR (CDCl₃, 500 MHz) δ 7.79 (s, 1H), 7.64 (s, 1H), 7.52 (d, 1H), 7.24 (dd, 2H), 7.13 (d, 1H), 7.01 (t, 2H), 6.89 (s, 1H), 6.73 (s, 1H), 6.20 (br s, 1H), 4.82 (s, 2H), 3.82 (s, 3H), 3.72 (d, 1H), 2.23 (s, 3H); MS (M+1)⁺m/z calcd for C₂₂H₂₀FN₅O₂⁺=406.2, found m/z=406.2. MW 405.4, Retention Time (min) 2.11, 2.29 (some TFA salt observed).

The following compounds were produced using a method similar to Method B, with heating at 60° C. in a sealed reaction vessel or in a microwave reactor at 140° C. for 30 minutes used in some cases.

| | | | DATA | |
|---|---|---|---|---|
| | | | MS(ESI) | Retention |
| Example | Structure | MW | m/z | Time (min) |
| B2 | 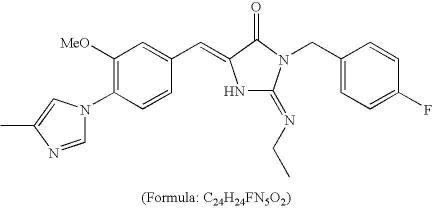 | 433.5 | 434.2 | 2.59 |
| B3 | 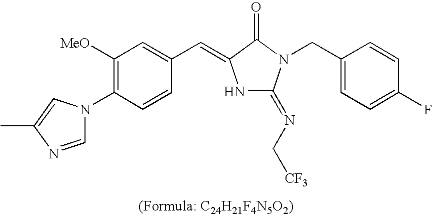 | 487.4 | 488.3 | 2.86 |
| B4 | 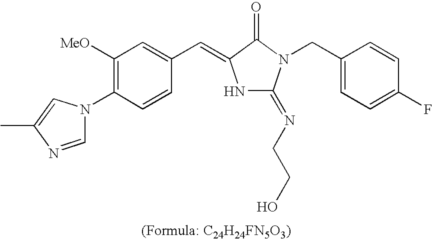 | 449.5 | 450.2 | 2.48 |
| B5 | 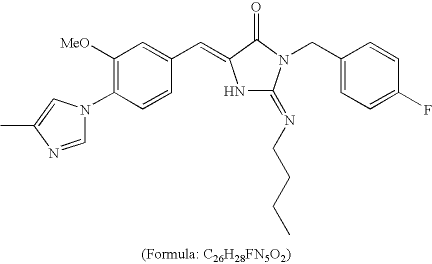 | 461.5 | 462.3 | 3.03 |
| B6 | 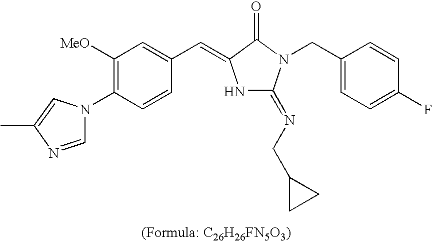 | 459.5 | 460.3 | 2.97 |
| B7 | 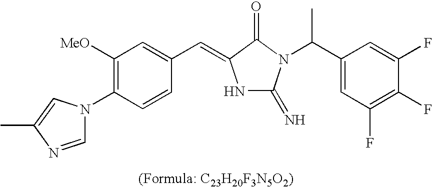 | 455.4 | 456.3 | 2.69 |
| B8 | 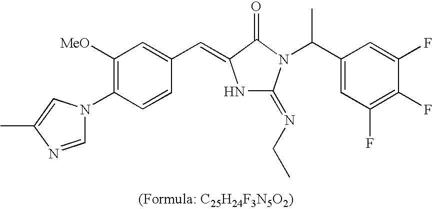 | 483.5 | 484.3 | 2.99 |
| B9 | 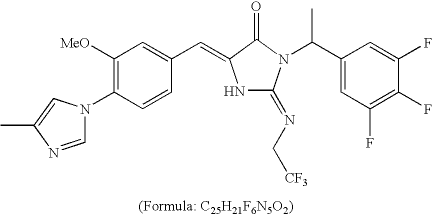 | 537.5 | 538.2 | 3.31 |
| B10 | 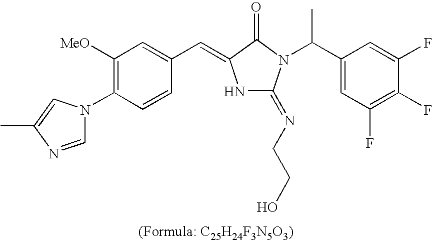 | 499.5 | 500.3 | 3.03 |
| B11 | 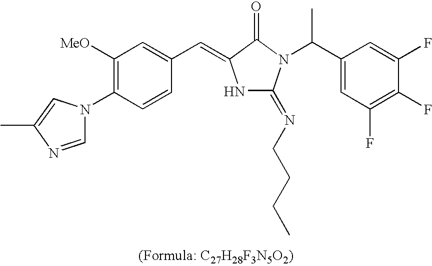 | 511.5 | 512.3 | 3.54 |
| (+)-B11 | 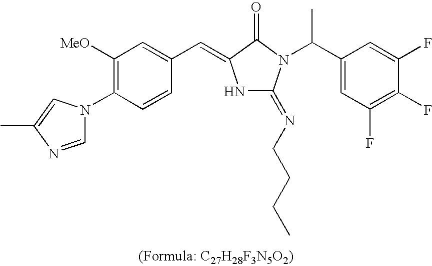 | 511.5 | 512.3 | 3.58 |
| (−)-B11 | 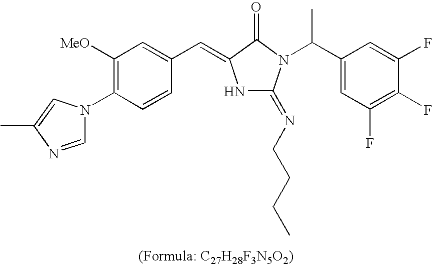 | 511.5 | 512.3 | 3.58 |
| B12 | 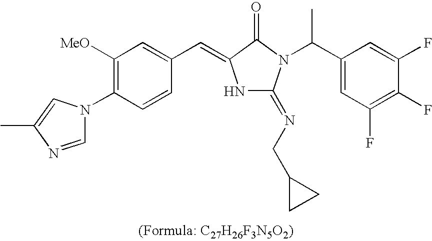 | 509.5 | 510.3 | 3.44 |
| B13 | 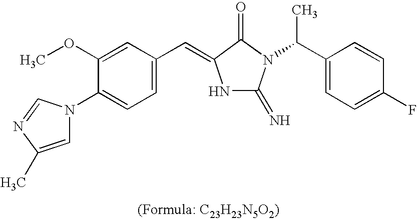 | 419.4 | 420.2 | 2.32 |
| B14 | 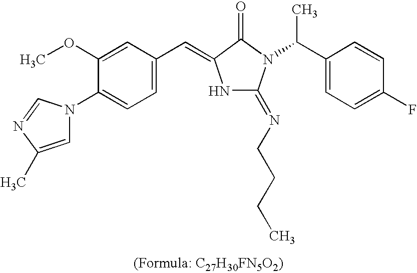 | 475.5 | 476.3 | 3.40 |
| B15 | 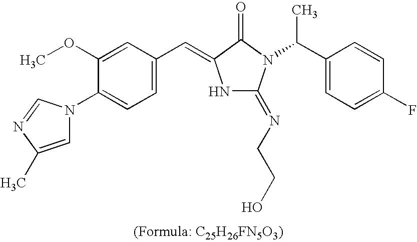 | 463.5 | 464.3 | 2.78 |
| B16 | 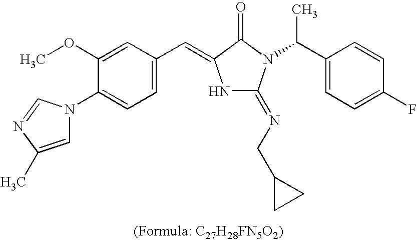 | 473.5 | 474.3 | 3.28 |
| B17 | 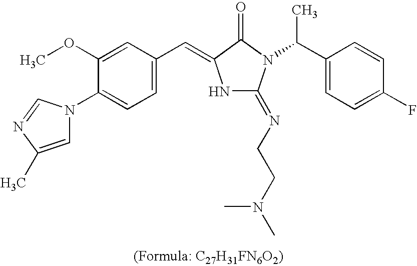 | 490.5 | 491.3 | 3.42 |
| B18 | 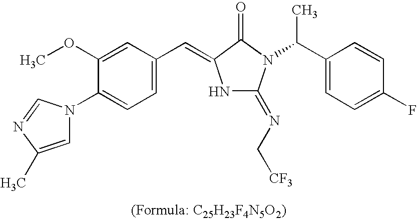 | 501.4 | 502.3 | 3.42 |
| B19 | 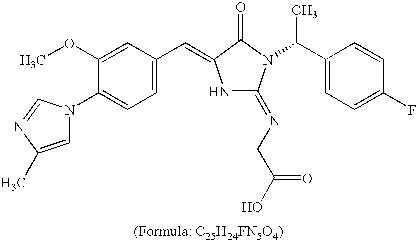 | 477.4 | 478.3 | 3.09 |
| B20 | 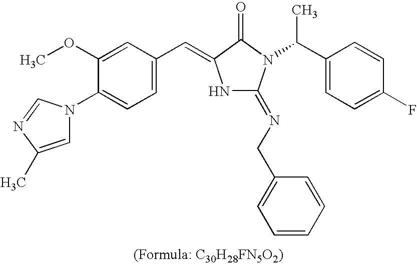 | 509.5 | 510.3 | 3.46 |
| B21 | 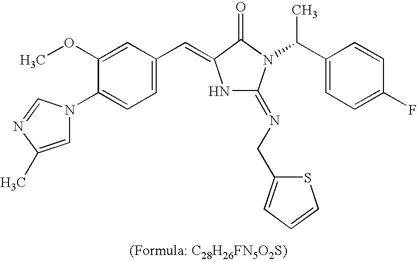 | 515.6 | 516.3 | 3.63 |
| B22 | 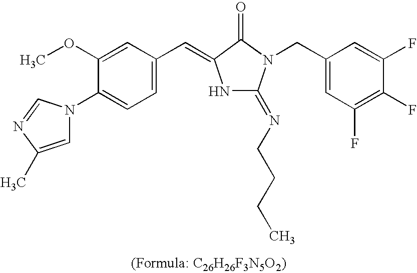 | 497.5 | 498.3 | 3.13 |
| B23 | 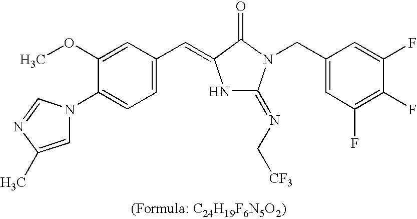 | 523.4 | 524.3 | 3.16 |

### Method C

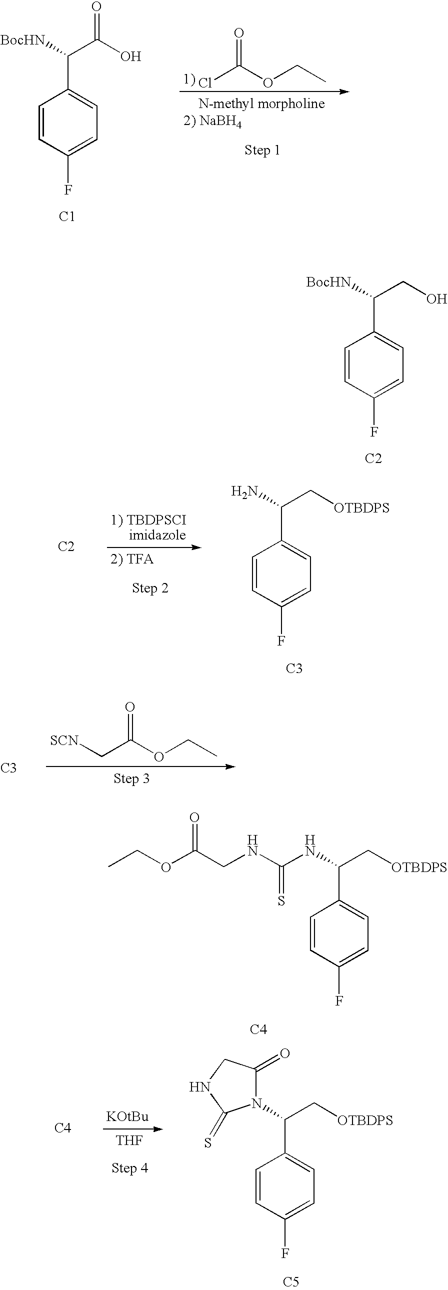

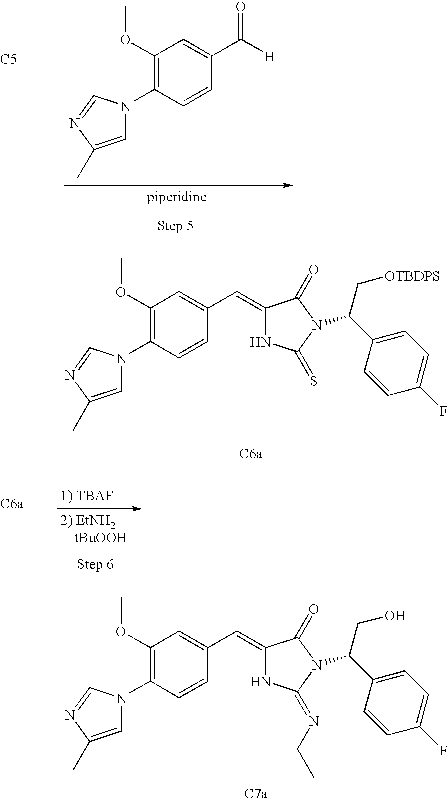

### Method C, Step 1

To a solution of (S)—N-Boc-4-fluorophenylglycine (2.0 g, 7.4 mmol) in tetrahydrofuran (20 mL) was added N-methylmorpholine (3.25 ml, 29.6 mmol) followed by ethyl chloroformate (1.4 mL, 14.8 mmol) and the reaction was stirred 30 min at RT then diluted with dichloromethane and water, extracted with dichloromethane, dried over sodium sulfate and concentrated. The residue was purified by chromatography over silica gel (eluted with hexanes/ethyl acetate 99:1 to ethyl acetate) to provide 1.76 g of an oil intermediate. To this intermediate (1.76 g, 5.2 mmol) in methanol (20 mL) at 0° C. was added sodium borohydride (0.4 g, 10.4 mmol) and the reaction was stirred 1 h at RT. The final mixture was worked-up with dichloromethane and brine to provide 1.23 g (72%) of (S)-tert-butyl 1-(4-fluorophenyl)-2-hydroxyethylcarbamate C2.

### Method C, Step 2

To a solution of C2 from Step 1 (1.23 g, 4.8 mmol) and imidazole (655 mg, 9.60 mmol) in DMF (10 mL) was added t-butyl chloro-diphenylsilane (1.95 mL, 7.60 mmol). The reaction was stirred overnight at RT then 12 h at 80° C. The final mixture was worked up with ether and half-concentrated aqueous brine to provide 1.70 g (90%) of oil intermediate. To this oil (1.70 g, 3.44 mmol) in dichloromethane (10 mL) was added TFA (1 mL) and the reaction was stirred 1 h at RT. The final mixture was diluted with 0.5N NaOH, extracted with dichloromethane and ethyl acetate, dried over sodium sulfate and concentrated to give 1.30 g of (S)-2-(tert-butyldiphenylsilyloxy)-1-(4-fluorophenyl)ethanamine C3.

### Method C, Step 3

To a solution of product C3 from Step 2 (1.30 g, 3.30 mmol) in dichloromethane (10 mL) was added ethyl isothiocyanatoacetate (0.5 mL, 3.6 mmol) followed by triethylamine (0.56 mL, 4.0 mmol). The reaction was stirred at RT overnight then worked-up in water and dichloromethane. The residue was purified by chromatography over silica gel (eluted with hexanes/ethyl acetate 99:1 to 50:50) to provide 1.21 g (70%) of (S)-ethyl 6-(4-fluorophenyl)-2,2-dimethyl-3,3-diphenyl-8-thioxo-4-oxa-7,9-diaza-3-silaundecan-11-oate C4.

### Method C, Step 4

To a solution of product C4 from Step 3 (1.21 g, 2.25 mmol) in tetrahydrofuran (10 mL) at 0 C was added tBuOK 1N in tetrahydrofuran (2.50 mL, 2.50 mmol). The reaction was worked up after 30 min by adding water and extracting with dichloromethane. The residue was purified by chromatography over silica gel (eluted with hexanes/ethyl acetate 99:1 to 50:50) to provide 720 mg (66%) of (S)-3-(2-(tert-butyldiphenylsilyloxy)-1-(4-fluorophenyl)ethyl)-2-thioxoimidazolidin-4-one C5.

### Method C, Step 5

To a solution of product C5 from Step 4 (720 mg, 1.46 mmol) and 3-methoxy-4-(4-methyl-1H-imidazol-1-yl)benzaldehyde (348 mg, 1.61 mmol) in ethanol (10 mL) was added piperidine (0.32 mmol, 3.20 mmol). The reaction was stirred at reflux overnight then concentrated. The residue was diluted with water and ethyl acetate, washed with water and brine, dried over sodium sulfate and concentrated. The residue was purified by chromatography over silica gel (eluted with hexanes/ethyl acetate 99:1 to ethyl acetate) to provide 850 mg (85%) of (S,Z)-3-(2-(tert-butyldiphenylsilyloxy)-1-(4-fluorophenyl)ethyl)-5-(3-methoxy-4-(4-methyl-1H-imidazol-1-yl)benzylidene)-2-thioxoimidazolidin-4-one C6a.

### Method C, Step 6

To a solution of product C6a from Step 5 (100 mg, 0.145 mmol) in tetrahydrofuran (2 mL) was added tetrabutyl ammonium fluoride 1N in tetrahydrofuran (0.36 mL, 0.36 mmol) and the reaction was stirred overnight at RT then 3 h at 45° C. The final mixture was worked up with water and ethyl acetate then purified by chromatography over silica gel (eluted with hexanes/ethyl acetate 70:30 to ethyl acetate) to provide 45 mg of intermediate alcohol C6b. LCMS (M+1⁺) m/z calcd for C₂₃H₂₂FN₄O₃S=453.1, found m/z=453.2; retention time=2.68 min.

To a solution of this intermediate alcohol C6b (40 mg, 0.088 mmol) in methanol (1 mL) was added tert-butylhydroperoxide (30 uL, 0.27 mmol) followed by 2N triethylamine in methanol (0.14 mL, 0.27 mmol) and the reaction was stirred at RT for 1 h. The final mixture was concentrated and purified over reverse-phase HPLC using 5% methanol/dichloromethane/NH₄OH system to provide 4.8 mg of (2E,5Z)-2-(ethylimino)-3-((S)-1-(4-fluorophenyl)-2-hydroxyethyl)-5-(3-methoxy-4-(4-methyl-1H-imidazol-1-yl)benzylidene)imidazolidin-4-one C7a. ¹H NMR (CDCl₃ 400 MHz) δ 8.35-8.5 (s, 1H), 8.23 (s, 1H), 7.85 (s, 1H), 7.41-7.43 (d, 1H), 7.30-7.33 (m, 2H), 7.17-7.19 (d, 1H), 7.03-7.07 (m, 2H), 6.93 (s, 1H), 6.62 (s, 1H), 5.6-6.0 (s, 1H), 5.52 (s, 1H), 4.50-4.60 (m, 1H), 4.18-4.23 (m, 1H), 3.85 (s, 3H), 3.42-3.44 (q, 2H), 2.30 (s, 3H), 1.10-1.13 (t, 3H); LCMS (M+1⁺) m/z calcd for C₂₅H₂₇FN₅O₃⁺=464.2, found m/z=464.3; retention time=2.55 min.

The following compounds were produced using a similar method:

| | | | DATA | Retention |
|---|---|---|---|---|
| | | | MS(ESI) | Time |
| Example | Structure | MW | m/z | (min) |
| C7b | 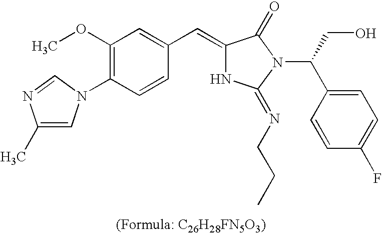 | 477.5 | 478.3 | 2.58 |
| C7c | 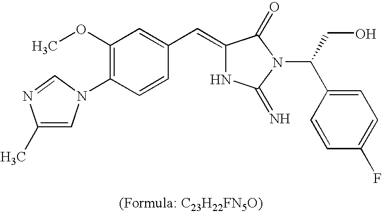 | 435.5 | 436.2 | 2.20 |
| C7d | 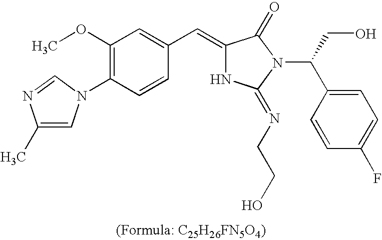 | 479.5 | 480.3 | 2.38 |
| C7e | 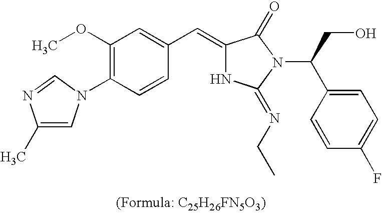 | 463.5 | 464.0 | 1.63 |
| C7f | 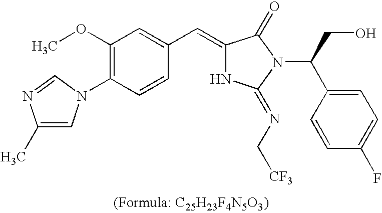 | 517.5 | 518.3 | 3.21 |

### Method D

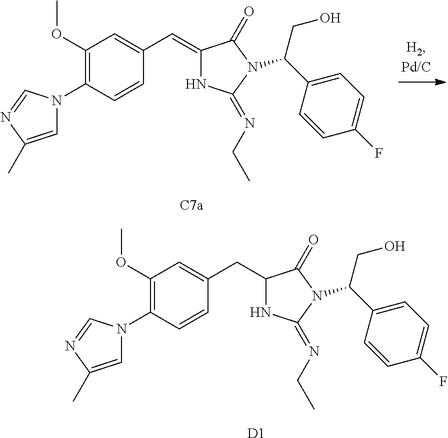

(E)-2-(ethylimino)-3-(S)-1-(4-fluorophenyl)-2-hydroxyethyl)-5-(3-methoxy-4-(4-methyl-1H-imidazol-1-yl)benzyl)imidazolidin-4-one, D1 To a solution of iminohydantoin C7a (6 mg, 0.013 mmol) in methanol (1 ml) was added palladium on carbon (1 mg) then the mixture was degassed several times with hydrogen gas via balloon. The reaction was stirred overnight at room temperature under hydrogen balloon pressure. The final mixture was filtered through a celite plug, washed with 20 ml methanol, then dried over sodium sulfate and concentrated. The residue was purified by preparatory TLC over silica gel (eluting with 90:10 dichloromethane/methanol) to provide 1.5 mg of D1. ¹H NMR (CDCl₃ 400 MHz) δ 7.7-7.6 (s, 1H), 7.38-7.2 (s, 1H), 7.1-6.9 (m, 31-1), 6.9-6.85 (m, 2H), 6.81-6.79 (m, 1H), 6.7-6.65 (s, 1H), 6.3-6.25 (d, 2H), 4.5-4.4 (m, 1H), 4.2-4.1 (m, 1H), 3.71 (s, 3H), 3.65-3.6 (m, 2H), 3.4-3.2 (m, 3H), 3.1-3.2 (m, 1H), 2.30 (s, 3H), 1.10-1.13 (t, 3H); LCMS (M+1⁺) m/z calcd for C₂₅H₂₉FN₅O₃⁺=466.2, found m/z=466.3; retention time=1.84 min.

| | | | DATA | Retention |
|---|---|---|---|---|
| | | | MS(ESI) | Time |
| Example | Structure | MW | m/z | (min) |
| D1 | 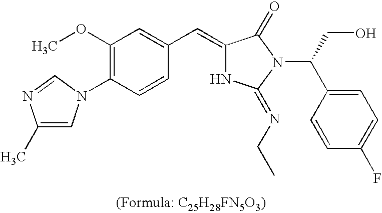 | 465.5 | 466.3 | 1.84 |

### Method E

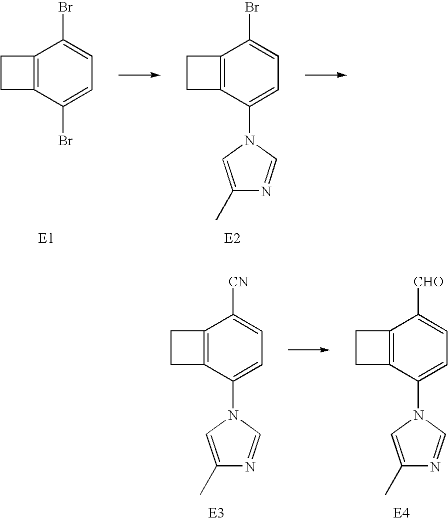

Compound E1 is obtained using a literature method by K. Walker, L., Markoski and J. Moore Synthesis, 1992, 1265.

### Method B, Step 1

To a solution of E1 (0.11 mmol) in dry 0.5 mL will be added 4-methyl imidazole (5 eq, 0.546 mmol, 44 mg), Cu₂O (0.4 equiv, 0.044 mmol, 6 mg), 4,7-dimethoxyl-1,8-phenanthracene (0.4 equiv, 0.044 mmol, 10 mg), Cs₂CO₃ (1.4 equiv, 0.154 mmol, 50 mg) and PEG (40 mg). The resulting solution will be degassed and heated at 110° C. for 40 h to give compound E1 after purification.

### Method B, Step 2

A procedure from P. Schirch and V. Bockclheide is adapted (J. Amer. Chem. Soc. 1981, 103, 6873). To a solution of E2 (1.5 g) will be added 5.0 eq of cuprous cyanide in 100 ml of N-methyl-2-pyrrolidinone. The mixture will be heated at 115° C. with stirring under nitrogen to give E3 after workup and purification.

### Method B, Step 3

To a 140 mg of E3 in ether will be added 1 eq of DiBAL in hexane. After 1 h, 5 mL of MeOH will be added and the mixture will be poured into ice water followed by acidification with 10% HCl and extraction with ether. The organic layers will be combined and solvent evaporated to give a residue which will be chromatographed to give compound E4.

The following intermediates will be synthesized using method similar to Method E:

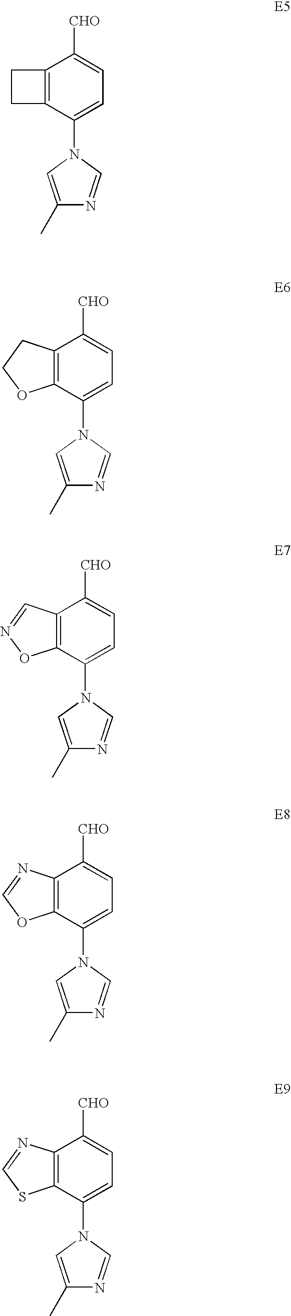

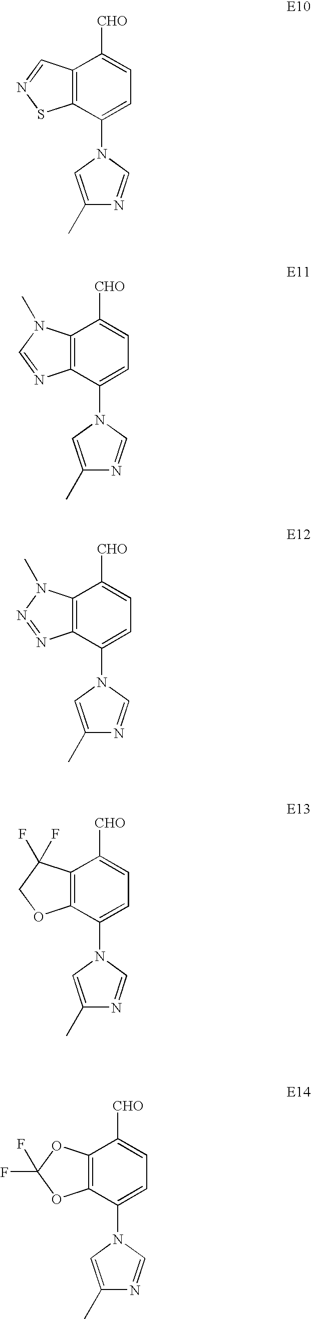

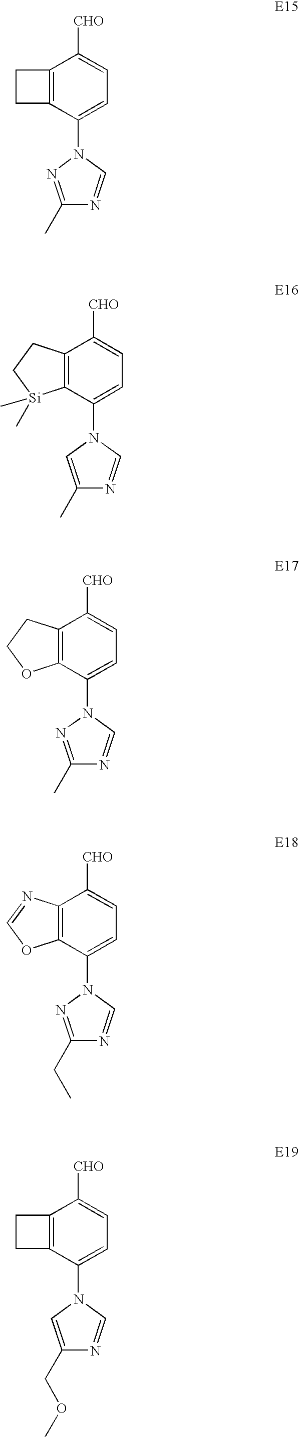

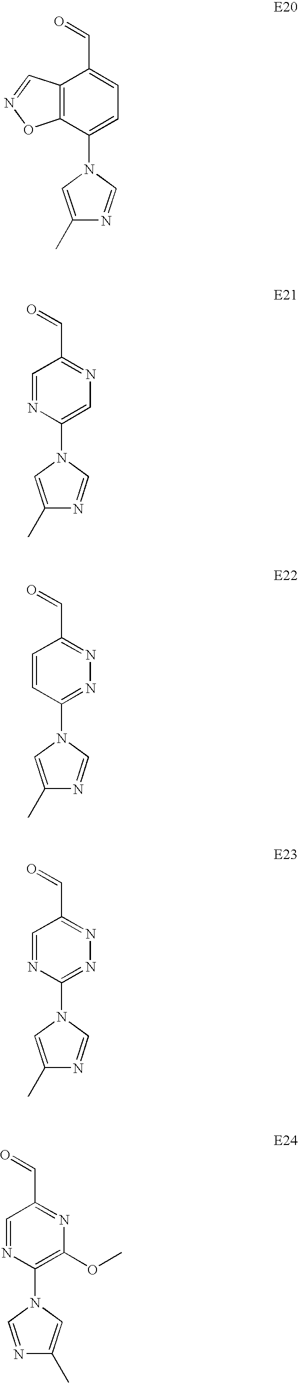

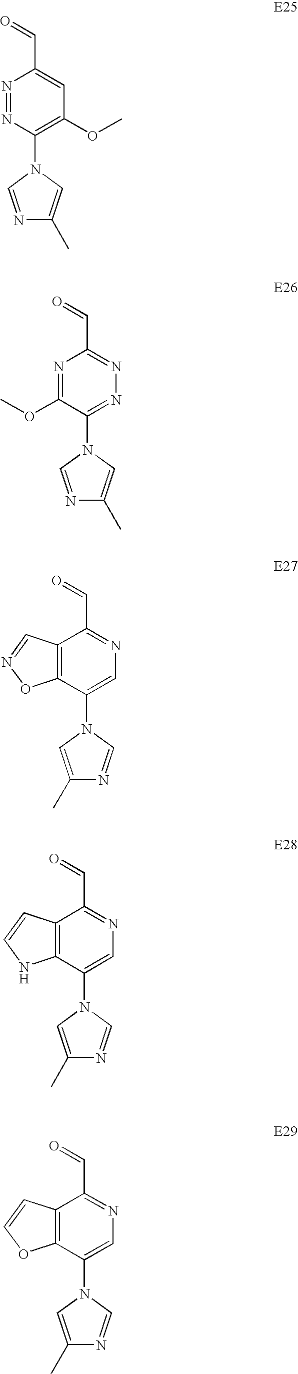

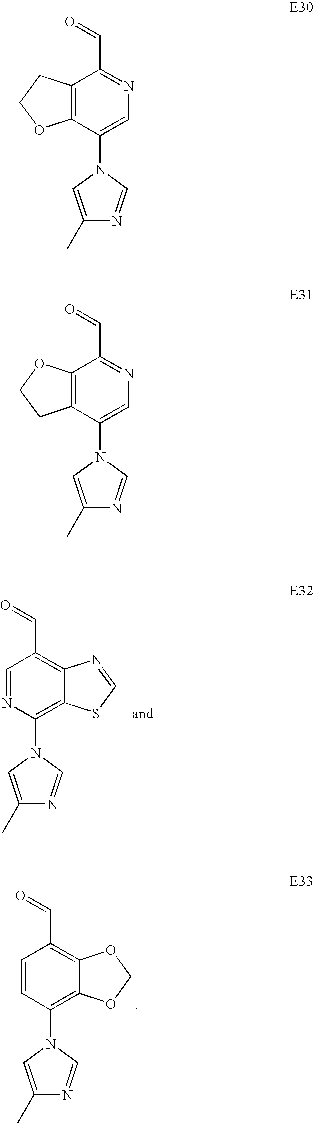

Intermediates useful for the preparation of compounds having —SF₅ and —OSF₅ groups are prepared from the reactions below as well as techniques well known in the art.

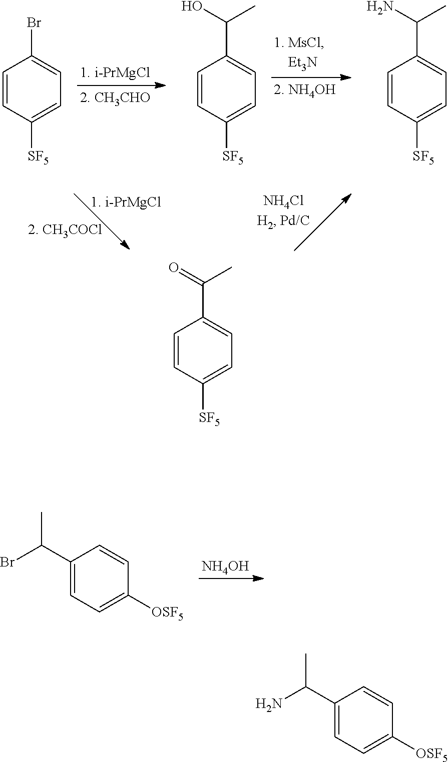

Compounds having —Si(R¹⁵)₃ (such as, for example, —Si(CH₃)₃) groups, or other —SF₅ substituted groups, or other —OSF₅ substituted groups are prepared following procedures similar to those above, as well as techniques well known in the art.

### Assay:

Secretase Reaction and Aβ Analysis in Whole Cells: HEK293 cells overexpressing APP with Swedish and London mutations were treated with the specified compounds for 5 hour at 37° C. in 100 ml of DMEM medium containing 10% fetal bovine serum. At the end of the incubation, total Aβ, Aβ40 and Aβ42 were measured using electrochemiluminescence (ECL) based sandwich immunoassays. Total Aβ was determined using a pair of antibodies TAG-WO2 and biotin-4G8, Aβ40 was identified with antibody pairs TAG-G2-10 and biotin-4G8, while Aβ42 was identified with TAG-G2-11 and biotin-4G8. The ECL signal was measured using Sector Imager 2400 (Meso Scale Discovery).

MS Analysis of Aβ Profile: Aβ profile in conditioned media was determined using surface enhanced laser desorption/ionization (SELDI) mass spectrometry. Conditioned media was incubated with antibody WO2 coated PS20 ProteinChip array. Mass spectra of Aβ captured on the array were read on SELDI ProteinChip Reader (Bio-Rad) according to manufacture's instructions.

CSF Aβ Analysis: Aβ in rat CSF was determined using MSD technology as described above. Aβ40 was measured using antibody pair Tag-G2-10 and biotin-4G8, while Aβ42 was measured using Tag-anti Aβ42 (Meso Scale Discovery) and biotin-4G8. The ECL signal was measured using Sector Imager 2400 (Meso Scale Discovery).

Matrix-assisted laser desorption/ionization mass spectrometric (MALDI MS) analysis of Aβ is performed on a Voyager-DE STR mass spectrometer (ABI, Framingham, Mass.). The instrument is equipped with a pulsed nitrogen laser (337 nm). Mass spectra are acquired in the linear mode with an acceleration voltage of 20 kV. Each spectrum presented in this work represents an average of 256 laser shots. To prepare the sample-matrix solution, 1 μL of immunoprecipitated Aβ sample is mixed with 3 μL of saturated a-cyano-4-hydroxycinnamic acid solution in 0.1% TFA/acetonitrile. The sample-matrix solution is then applied to the sample plate and dried at ambient temperature prior to mass spectrometric analysis. All the spectra are externally calibrated with a mixture of bovine insulin and ACTH (18-39 clip).

Compounds A9a to A9u, B1 to B11, (+)-B11, (−)-B11, B 12 to B23, C7a to C7f, and D1 had an Aβ42IC₅₀ in the range of about 46 to about 15781 nM.

Compounds A9a to A9u, B1 to B11, (+)-B11, (−)-B11, B 12 to B23, C7a to C7f, and D1 had an Aβ Total IC₅₀ in the range of about 778 to about 89,953 nM.

Compounds A9m, A9r, A9t, A9u, B2, B4, B5, B6, B7, B8, B10, B11, (+)-B11, (−)-B11, B12, B13, B14, B15, B18, B22, and B23 had an Aβ42IC₅₀ in the range of about 46 to about 94 nM, and an Aβ Total IC₅₀ in the range of about 778 to about 20,000 nM.

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

1. A compound of the formula: 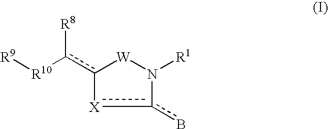
or a pharmaceutically acceptable salt thereof, wherein:
R¹, R⁸, R⁹, R¹⁰, B, W and X are independently selected;
the dashed lines (- - -) represent optional bonds, provided that either the optional bond to X is present, or the optional bond to B is present, but not both;
B is selected from the group consisting of: H, alkoxy, alkyl, cycloalkyl, heterocycloalkyl, alkoxyalkyl-, hydroxyalkyl-, —OR^{15a}, ═O, ═S, ═N—O-alkyl, and 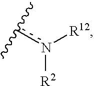
provided that:
(a) when the optional bond to N is present (i.e., the optional bond to B is present) then the R¹² substituent is absent, and
(b) provided that when X is —N(R¹⁴)—, or ═N—, and W is —C(O)—, then B is not ═O or ═S;
W is selected from the group consisting of: —C(O)— and —S(O)₂—;
X is selected from the group consisting of:
(a) —N(R¹⁴)— and —C(R⁶)(R⁷)— when the optional bond to X is present, and
(b) —N═, —C(R⁶)═, and —C(R⁷)=when the optional bond to X is absent;
when the optional bond in the moiety. 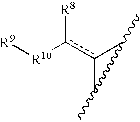
is present then said moiety is: 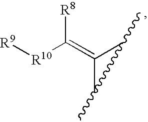
and when the optional bond in the moiety: 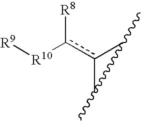
is absent then said moiety is selected from the group consisting of: 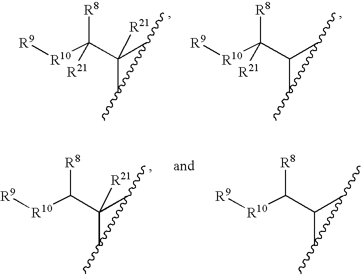
wherein each R²¹ is independently selected;
R¹ is selected from the group consisting of H, alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl-, and wherein each of said alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl- R¹ groups are optionally substituted with 1-5 independently selected R²¹ substituents;
R² is selected from the group consisting of H, alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, —CN, —C(O)R¹⁵, —C(O)N(R¹⁶)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶ and —P(O)(OR¹⁵)(OR¹⁶), and wherein each of said alkyl, alkenyl, alkynyl, cycloalkyl, cycloalkylalkyl-, cycloalkenyl, heterocycyl, heterocyclylalkyl-, aryl, arylalkyl-, heteroaryl, and heteroarylalkyl- R² groups are optionally substituted with 1-5 independently selected R²¹ substituents;
R⁶ is selected from the group consisting of H, halo, alkyl, alkenyl, alkynyl, aryl, arylalkyl, alkylaryl-, cycloalkyl, cycloalkylalkyl-, heteroaryl, heteroarylalkyl-, heterocyclyl and heterocyclyalkyl-, wherein each of said alkyl, alkenyl, alkynyl, aryl, arylalkyl-, alkylaryl-, cycloalkyl cycloalkylalkyl-, heteroaryl, heteroarylalkyl-, heterocyclyl and heterocyclyalkyl- R⁶ groups are optionally substituted with 1-5 independently selected R²¹ substituents;
R⁷ is selected from the group consisting of H, halo, alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl-, wherein each of said alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl- R⁷ groups are optionally substituted with 1-5 independently selected R²¹ substituents;
R⁸ is selected from the group consisting of H, halo, alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl-, with each of said alkyl, alkenyl, alkynyl, aryl, arylalkyl-, alkylaryl-, cycloalkyl, cycloalkylalkyl-, heteroaryl- heteroarylalkyl-, heterocyclyl and heterocyclyalkyl- R⁸ groups are optionally substituted with 1-3 independently selected R²¹ substituents;
R⁹ is selected from the group consisting of alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl-, wherein each of said alkyl, alkenyl, alkynyl, aryl, arylalkyl-, alkylaryl-, cycloalkyl, cycloalkylalkyl-, heteroaryl, heteroarylalkyl-, heterocyclyl and heterocyclyalkyl- R⁸ groups are optionally substituted with 1-3 independently selected R²¹ substituents;
R¹⁰ is selected from the group consisting of a bond, alkyl, alkenyl, alkynyl, aryl, arylalkyl-, alkylaryl-, cycloalkyl, cycloalkylalkyl-, heteroaryl, heteroarylalkyl-, heterocyclyl, heterocyclyalkyl-, 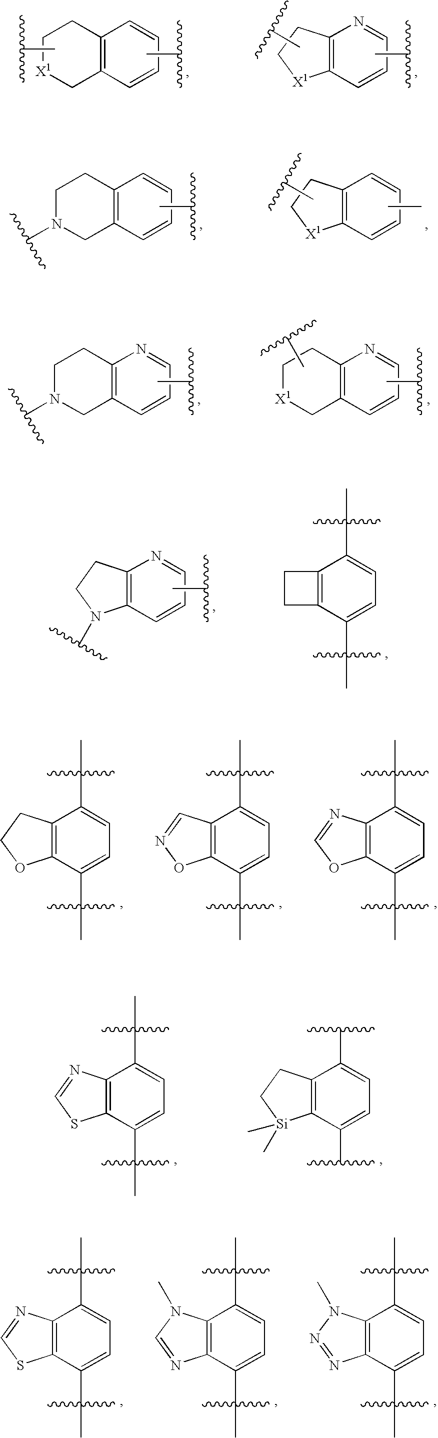 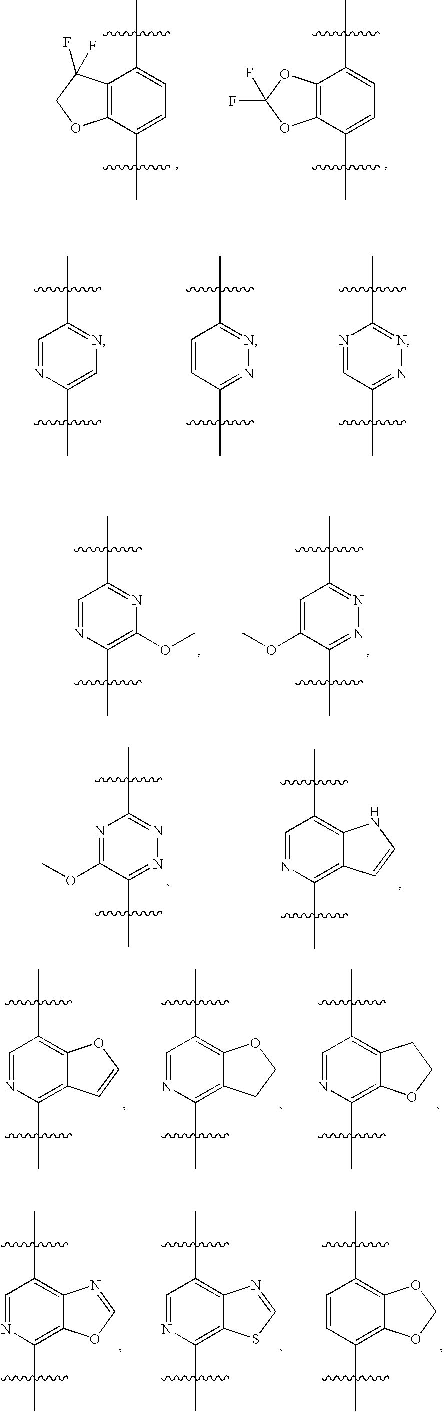 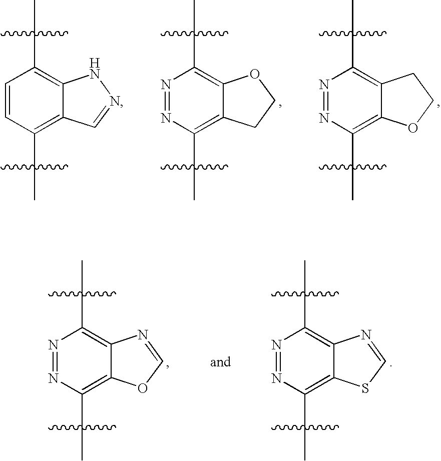
wherein X¹ is O, N(R¹⁴) or S;
wherein each of said R¹⁰ substituents (excluding the R¹⁰ bond) is optionally substituted with 1-3 independently selected R²¹ substituents;
R¹² is independently selected from the group consisting of H, alkyl, alkenyl, alkynyl, cycloalkyl, cycloalkenyl, heterocyclyl, heterocyclylalkyl-, aryl, arylalkyl-, heteroaryl, heteroarylalkyl-, —CN, —C(O)R¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶ and —P(O)(OR¹⁵)(OR¹⁶), and wherein each of said alkyl, alkenyl, alkynyl, cycloalkyl, cycloalkylalkyl-, cycloalkenyl, heterocyclyl, heterocyclylalkyl-, aryl, arylalkyl-, heteroaryl, and heteroarylalkyl- R¹² groups are optionally substituted with 1-5 independently selected R²¹ substituents;
each R¹⁴ be the same or different, each being independently selected from the group consisting of H, alkyl, alkenyl, alkynyl, cycloalkyl, cycloalkylalkyl-, cycloalkenyl, heterocyclyl, heterocyclylalkyl-, aryl, arylalkyl, heteroaryl, heteroarylalkyl-, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —S(O)R¹⁵, —S(O)₂R¹⁵, —C(═NOR¹⁵)R¹⁶, and —P(O)(OR¹⁵)(OR¹⁶), and wherein each of said alkyl, alkenyl, alkynyl, cycloalkyl, cycloalkylalkyl-, cycloalkenyl, heterocyclyl, heterocyclylalkyl-, aryl, arylalkyl-, heteroaryl, and heteroarylalkyl- R¹⁴ groups are optionally substituted with 1-5 independently selected R²¹ substituents;
R^{15a} is independently selected from the group consisting of alkyl, cycloalkyl, cycloalkylalkyl-, heterocyclyl, heterocyclylalkyl-, arylalkyl-, heteroarylalkyl-, arylcycloalkyl-, arylheterocyclyl-, (R¹⁸)ₙ-alkyl-, (R¹⁸)ₙ-cycloalkyl-, (R¹⁸)ₙ-cycloalkylalkyl-, (R¹⁸)ₙ-heterocyclyl-, (R¹⁸)ₙ-heterocyclylalkyl-, (R¹⁸)ₙ-aryl-, (R¹⁸)ₙ-arylalkyl-, (R¹⁸)ₙ-heteroaryl- and (R¹⁸)ₙ-heteroarylalkyl-, wherein n is 1 to 5;
R¹⁵ is independently selected from the group consisting of H, alkyl, cycloalkyl, cycloalkylalkyl-, heterocyclyl, heterocyclylalkyl-, heteroarylalkyl-, arylcycioalkyl-, arytheterocyclyl- (R¹⁸)ₙ-cycloalkyl-, (R¹⁸)ₙ-cycloalkalkyl-, (R¹⁸)ₙ-heterocyclyl-, (R¹⁸)ₙ-heterocyclylalkyl-, (R¹⁸)ₙ-aryl-, (R¹⁸)ₙ-arylalkyl-, (R¹⁸)ₙ-heteroaryl- and (R¹⁸)ₙ-heteroarylalkyl-, wherein n is 1 to 5;
R¹⁶ and R¹⁷ are independently selected from the group consisting of H, alkyl, alkenyl, alkynyl, cycloalkyl, cycloalkylalkyl-, heterocyclyl, heterocyclylalkyl aryl, arylalkyl-, heteroaryl, heteroarylalkyl-, arylcycloalkyl-, arylheterocyclyl-, (R¹⁸)ₙ-alkyl-, (R¹⁸)ₙ-cycloalkyl-, (R¹⁸)ₙ-cycloalkylalkyl-, (R¹⁸)ₙ-heterocyclyl-, (R¹⁸)ₙ-heterocyclylalkyl-, (R¹⁸)ₙaryl-, (R¹⁸)ₙ-arylalkyl-, (R¹⁸)ₙ-heteroaryl- and (R¹⁸)ₙ-heteroaryalkyl-;
Each R¹⁸ is independently selected from the group consisting of alkyl, alkenyl, alkynyl, aryl, arylalkyl-, arylalkenyl-, arylalkynyl-, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR₁₉, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);
or, alternately, two R¹⁸ moieties on adjacent carbons can be linked together to form: 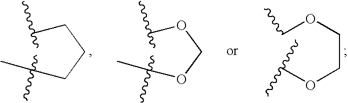
R¹⁹ is selected from the group consisting of: alkyl, cycloalkyl, aryl, arylalkyl- and heteroarylalkyl-;
R²⁰ is selected from the group consisting of: alkyl, cycloalkyl, aryl, halo substituted aryl, arylalkyl-, heteroaryl or heteroarylalkyl-;
Each R²¹ is independently selected from the group consisting of alkyl, alkenyl, alkynyl, cycloalkyl, cycloalkylalkyl-, cycloalkenyl, heterocyclyl, heterocyclylalkyl-, aryl, arylalkyl-, heteroaryl, heteroarylalkyl-, halo, —CN, —OR¹⁵, —C(O)R¹⁵, —C(O)OR¹⁵, —C(O)N(R¹⁵)(R¹⁶), —SF₅, —OSF₅, —Si(R¹⁵)₃ wherein each R¹⁵ is independently selected, —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), N(R¹⁵)S(O)R¹⁸, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶—CH₂—N(R¹⁵)C(O)OR¹⁶—S(O)R¹⁵, ═NOR¹⁵, —N₃, —NO₂ and —S(O)₂R¹⁵; and
wherein each of the alkyl, cycloalkenyl, cycloalkyl, cycloalkylalkyl-, heterocyclyl, heterocyclylalkyl-, aryl, arylalkyl, heteroaryl, heteroarylalkyl- alkenyl and alkynyl groups in R²¹ are optionally substituted by 1 to 5 R²² groups independently selected from the group consisting of alkyl, cycloalkyl, cycloalkenyl, heterocyclyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SF₅, —OSF₅, —Si(R¹⁵)₃ wherein each R¹⁵ is independently selected, —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶—N(R¹⁵)S(O)R¹⁶—N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵—NO₂, —S(O)R¹⁵ and —S(O)₂R¹⁵.

2. The compound of claim 1 wherein said R¹⁰ is selected from the group consisting of aryl and aryl substituted with one or more R²¹ groups, and said R⁹ group is selected from the group consisting of heteroaryl and heteroaryl substituted with one or more R²¹ groups, wherein each R²¹ is independently selected.

3-4. (canceled)

5. The compound of claim 1 wherein the R⁹-R¹⁰-moiety is: 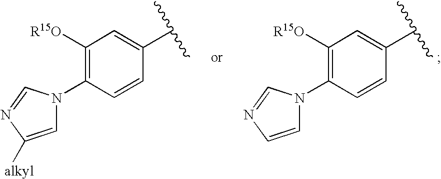
or wherein the R⁹-R¹⁰-moiety is:

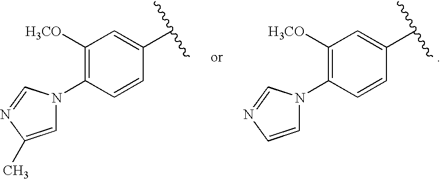

6. The compound of claim 1 wherein said R¹ group is: 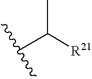
wherein R²¹ is unsubstituted or substituted with one or more independently selected R²² groups.

7. The compound of claim 1 wherein:
R¹ is an alkyl group substituted with one R²¹ group, and said R²¹ group is an aryl group; or
R¹ is an alkyl group substituted with one R²¹ group, and said R²¹ group is an aryl group, and said aryl is phenyl, and said alkyl group is methyl or ethyl; or
R¹ is an alkyl group substituted with one R²¹ group, and said R²¹ group is an aryl group, and said aryl group is substituted with one or more R²² groups; or
R¹ is an alkyl group substituted with one R²¹ group, and said R²¹ group is an aryl group, and said aryl group is substituted with one or more R²² groups wherein each R²² group is the same or different halo; or
R¹ is an alkyl group substituted with one R²¹ group, and said R²¹ group is an aryl group, and said aryl group is substituted with one or two R²² halo groups; or
R¹ is an alkyl group substituted with one R²¹ group, and said R²¹ group is an aryl group, and said aryl group is substituted with one or two R²² halo groups wherein the halo is F; or
R¹ is an alkyl group substituted with one R²¹ group, and said R²¹ group is an aryl group, and said aryl group is substituted with one or more R²² groups, and at least one R²² group is selected from the group consisting of: —SF₅, —OSF₅ and —Si(R¹⁵)₃, wherein each R¹⁵ is independently selected.

8. The compound of claim 1 wherein:
R¹ is an alkyl group substituted with one R²¹ group, and said R²¹ group is phenyl, and said phenyl is substituted with one or more R²² groups, and at least one R²² is selected from the group consisting of: —SF₅, —OSF₅ and —Si(R¹⁵)₃, wherein each R¹⁵ is independently selected; or
R¹ is an alkyl group substituted with one R²¹ group, and said R²¹ group is phenyl, and said phenyl is substituted with one or more R²² groups; or
R¹ is an alkyl group substituted with one R²¹ group, and said R²¹ group is phenyl, and said phenyl is substituted with one or more R²² groups, and each R²² group is the same or different halo; or
R¹ is an alkyl group substituted with one R²¹ group, and said R²¹ group is phenyl, and said phenyl is substituted with one two, or three R²² halo groups, and each R²² group is the same or different halo; or
R¹ is an alkyl group substituted with one R²¹ group, and said R²¹ group is phenyl, and said phenyl is substituted with one two, or three R²² F groups; or
R¹ is an alkyl group substituted with one R²¹ group, and said R²¹ group is phenyl, and said phenyl is substituted with one or two R²² halo groups, and each R²² group is the same or different halo; or
R¹ is an alkyl group substituted with one R²¹ group, and said R²¹ group is phenyl, and said phenyl is substituted with one or two R²² F groups; or
R¹is: 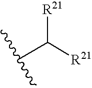
wherein one R²¹ is an unsubstituted or substituted alkyl group, and the other R²¹ is an unsubstituted or substituted phenyl group.

9. The compound of claim 1 wherein:
R¹ is an ethyl group substituted with one R²¹ group, and said R²¹ group is phenyl, and said phenyl is substituted with one or more R²² groups; or
R¹ is a methyl group substituted with one R²¹ group, and said R²¹ group is phenyl, and said phenyl is substituted with one or more R²² groups; or
R¹ is an ethyl group substituted with one R²¹ group, and said R²¹ group is phenyl, and said phenyl is substituted with one, two, or three R²² halo groups, and each R²² group is the same or different halo; or
R¹ is an ethyl group substituted with one R²¹ group, and said R²¹ group is phenyl, and said phenyl is substituted with one or two R²² halo groups, and each R²² group is the same or different halo; or
R¹ is a methyl group substituted with one R²¹ group, and said R²¹ group is phenyl, and said phenyl is substituted with one, two or three R²² halo groups, and each R²² group is the same or different halo; or
R¹ is a methyl group substituted with one R²¹ group, and said R²¹ group is phenyl, and said phenyl is substituted with one or two R²² halo groups, and each R²² group is the same or different halo; or
R¹ is an ethyl group substituted with one R²¹ group, and said R²¹ group is phenyl, and said phenyl is substituted with one, two, or three R²² F groups; or
R¹ is an ethyl group substituted with one R²¹ group, and said R²¹ group is phenyl, and said phenyl is substituted with one or two R²² F groups; or
R¹ is a methyl group substituted with one R²¹ group, and said R²¹ group is phenyl, and said phenyl is substituted with one, two or three R²² F groups; or
R¹ is a methyl group substituted with one R²¹ group, and said R²¹ group is phenyl, and said phenyl is substituted with one or two R²² F groups; or
R¹ is an ethyl group substituted with one R²¹ group, and said R²¹ group is phenyl, and said phenyl is substituted with one R²² halo group; or
R¹ is a methyl group substituted with one R²¹ group, and said R²¹ group is phenyl, and said phenyl is substituted with one R²² halo group; or
R¹ is an ethyl group substituted with one R²¹ group, and said R²¹ group is phenyl, and said phenyl is substituted with one R²² F group; or
R¹ is a methyl group substituted with one R²¹ group, and said R²¹ group is phenyl, and said phenyl is substituted with one R²² F group; or
R¹ is an ethyl group substituted with one R²¹ group, and said R²¹ group is phenyl, and said phenyl is substituted with two of the same or different R²² halo groups; or
R¹ is a methyl group substituted with one R²¹ group, and said R²¹ group is phenyl, and said phenyl is substituted with two of the same or different R²² halo groups; or
R¹ is an ethyl group substituted with one R²¹ group, and said R²¹ group is phenyl, and said phenyl is substituted with two R²² F groups; or
R¹ is a methyl group substituted with one R²¹ group, and said R²¹ group is phenyl, and said phenyl is substituted with two R²² F group; or
R¹ is an ethyl group substituted with one R²¹ group, and said R²¹ group is phenyl, and said phenyl is substituted with three of the same or different R²² halo groups; or
R¹ is a methyl group substituted with one R²¹ group, and said R²¹ group is phenyl, and said phenyl is substituted with three of the same or different R²² halo groups; or
R¹ is an ethyl group substituted with one R²¹ group, and said R²¹ group is said phenyl is substituted with three R²² F groups; or
R¹ is a methyl group substituted with one R²¹ group, and said R²¹ group is phenyl, and said phenyl is substituted with three R²² F group; or
R¹ is: 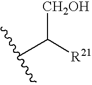
and R²¹ is unsubstituted phenyl or phenyl substituted with one or more independently selected R²² groups.

10. The compound of claim 1 wherein said R¹ is selected from the group consisting of:

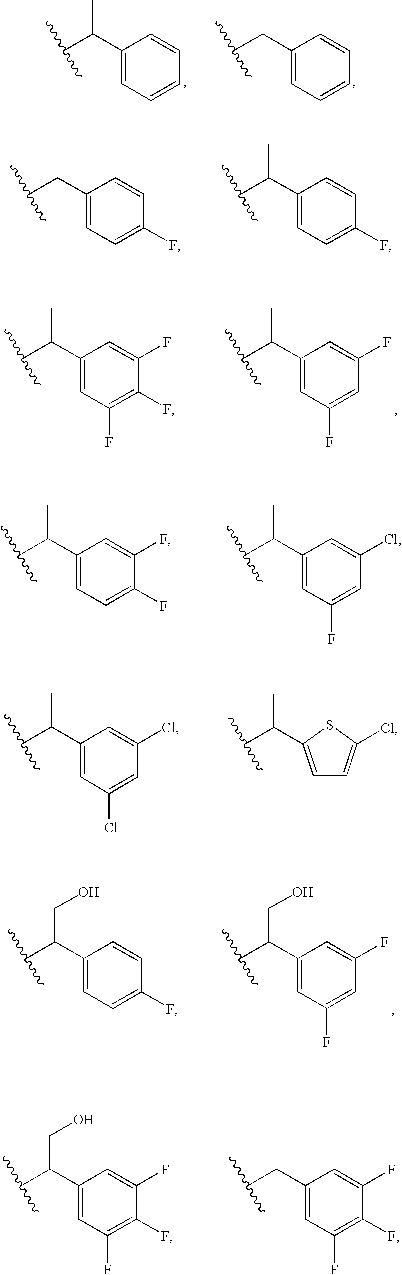

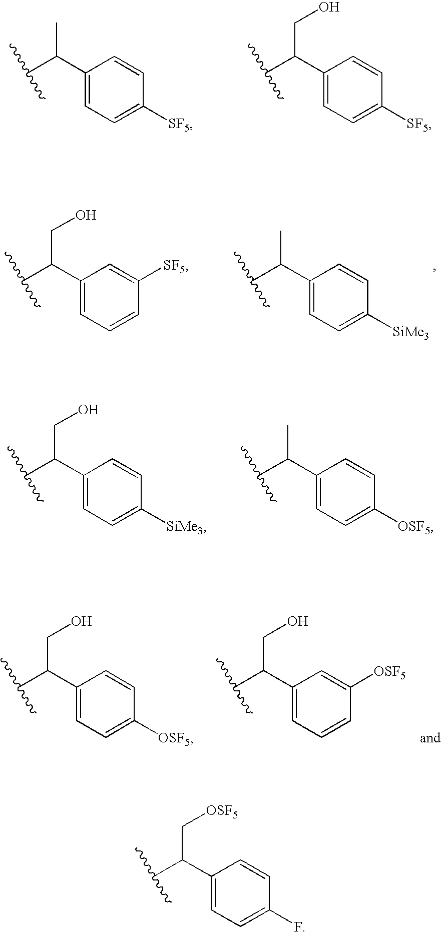

11. The compound of claim 1 wherein said R¹⁰ is selected from the group consisting of heteroaryl and heteroaryl substituted with one or more R²¹ groups, and said R⁹ group is selected from the group consisting of heteroaryl and heteroaryl substituted with one or more R²¹ groups, and wherein each R²¹ is independently selected.

12. The compound of claim 1 wherein:
(1)
R¹ is an alkyl group substituted with one R²¹ group, or
R¹ is an alkyl group substituted with one R²¹ group, and said R²¹ group is substituted with one or more independently selected R²² groups, and
R¹⁰ is selected from the group consisting of aryl and aryl substituted with one or more independently selected R²¹ groups, and
R⁹ is selected from the group consisting of heteroaryl and heteroaryl substituted with one or more independently selected R²¹ groups: or
(2)
R¹ is an alkyl group substituted with one phenyl, or
R¹ is an alkyl group substituted with one phenyl, and said phenyl is substituted with one or more independently selected R²² groups, and
R¹⁰ is selected from the group consisting of phenyl and phenyl substituted with one or more independently selected R²¹ groups, and
R⁹ is selected from the group consisting of imidazolyl and imidazolyl substituted with one or more independently selected R²¹ groups; or
(3)
R¹ is a methyl or ethyl group substituted with one phenyl, or
R¹ is a methyl or ethyl group substituted with one phenyl, and said phenyl is substituted with one or more independently selected halos, and
R¹⁰ is selected from the group consisting of phenyl and phenyl substituted with one or more independently selected —OR¹⁵ groups, and
R⁹ is selected from the group consisting of imidazolyl and imidazolyl substituted with one or more independently selected alkyl groups; or
(4)
R¹ is a methyl or ethyl group substituted with one phenyl, or
R¹ is an methyl or ethyl group substituted with one phenyl, and said phenyl is substituted with one or two independently selected halos, and
R¹⁰ is selected from the group consisting of phenyl and phenyl substituted with one or two independently selected —OR¹⁵ groups, wherein R¹⁵ is alkyl, and
R⁹ is selected from the group consisting of imidazolyl and imidazolyl substituted with one or two independently selected alkyl groups; or
(5)
R¹ is a methyl or ethyl group substituted with one phenyl, or
R¹ is an methyl or ethyl group substituted with one phenyl, and said phenyl is substituted with one or two F, and
R¹⁰ is selected from the group consisting of phenyl and phenyl substituted with one or two independently selected —OR¹⁵ groups, wherein R¹⁵ is methyl, and
R⁹ is selected from the group consisting of imidazolyl and imidazolyl substituted with one or two independently selected methyl groups; or
(6)
R¹ is a methyl or ethyl group substituted with one phenyl, or
R¹ is an methyl or ethyl group substituted with one phenyl, and said phenyl is substituted with one or two F, and
R¹⁰ is phenyl substituted with one —OR¹⁵ group, wherein R¹⁵ is methyl, and
R⁹ is selected from the group consisting of imidazolyl and imidazolyl substituted with one methyl group; or
(7)
R¹ is selected from the group consisting of: 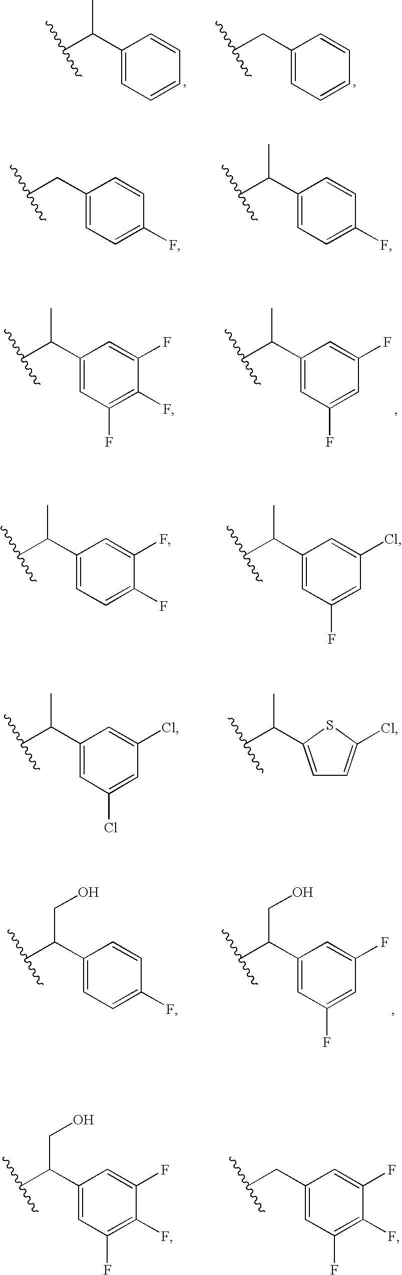 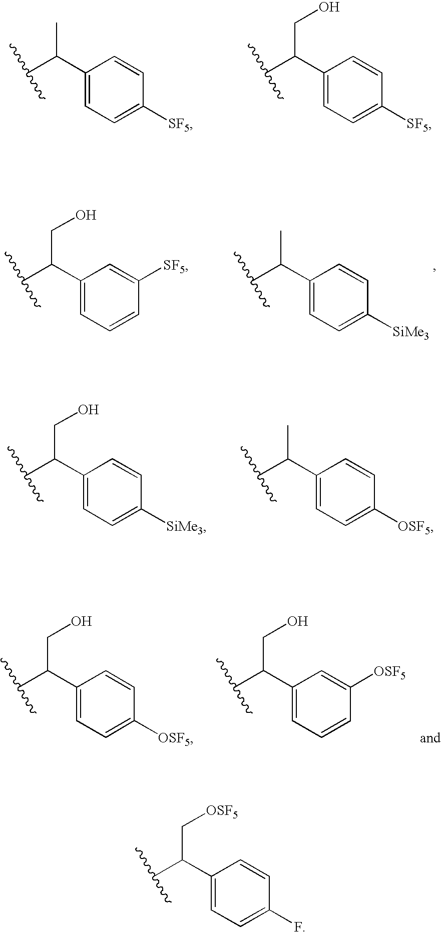
and wherein the R⁹-R¹⁰-moiety 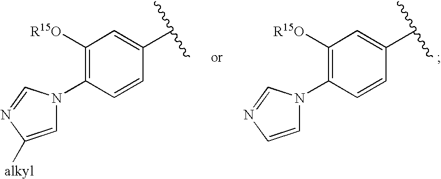
or
(8)
R¹ is selected from the group consisting of: 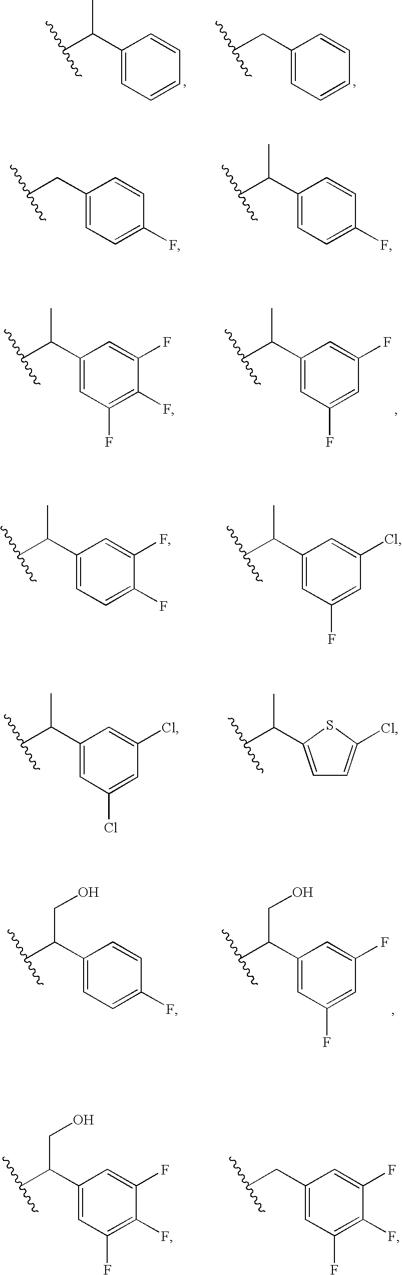 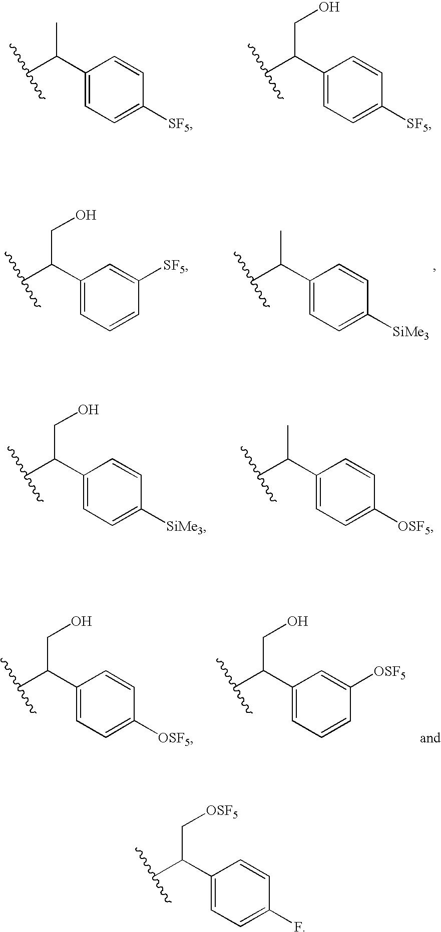
and wherein the R⁹-R¹⁰-moiety is:

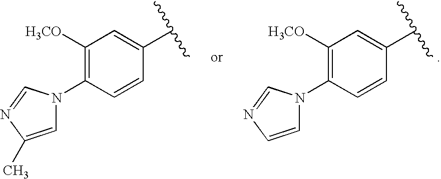

13. The compound of claim 10 wherein W is —C(O)—.

14-17. (canceled)

18. The compound of claim 1 wherein:
X is —N═ and the compound of formula (I) is IA; or
X is —N(R¹⁴)— and the compound of formula (I) is IB; or
X is —N═ and the compound of formula (I) is IC; or
X is —N(R¹⁴)— and the compound of formula (I) is ID; or
X is —N═ and the compound of formula (I) is IE; or
X is —N(R¹⁴)— and the compound of formula (I) is IF; or
X is —N═ and the compound of formula (I) is IG: or
X is —N(R¹⁴)— and the compound of formula (I) is IH; or
X is —N═ and the compound of formula (I) is II; or
X is —N(R¹⁴)— and the compound of formula (I) is IJ; or
X is —N═ and the compound of formula (I) is IK.

19. The compound of claim 1 wherein B is:
H; or
is selected from the group consisting of methoxy, ethoxy, propoxy, butoxy, pentoxy, and hexoxy; or
is selected from the group consisting of methyl, ethyl, propyl, butyl, pentyl, and hexyl; or
is selected from the group consisting of cyclopropyl, cyclobutyl, cyclopentyl, cyclohexyl, and cycloheptyl; or
piperidinyl and pyrrolidinyl; or
is selected from the group consisting of CH₃—O—CH₂—, CH₃—O—CH₂—CH₂—, CH₃—O—CH₂—CH₂—CH₂—, and CH₃—O—CH₂—CH₂—CH₂—CH₂—; or
is selected from the group consisting of HO—CH₂—, HO—CH₂—CH₂—CH₂—, HO—CH₂—CH₂—CH₂—, and HO—CH₂—CH₂—CH₂—CH₂-;
is —OR^{15a}; or
is ═O; or
is ═S; or
═N—O—CH₃; or
is selected from the group consisting of HO—CH₂—N═, HO—CH₂—CH₂—N═, HO—CH₂—CH₂—CH₂—N═, and HO—CH₂—CH₂—CH₂—CH₂—N═; or
is selected from the group consisting of ═NH, methoxy-N═, ethoxy-N═, propoxy-N═, butoxy-N═, pentoxy-N═, hexoxy-N═, methyl-N═, ethyl-N═, propyl-N═, butyl-N═, pentyl-N═, hexyl-N═, cyclopropyl-N═, cyclobutyl-N═, cyclopentyl-N═, cyclohexyl-N═, cycloheptyl-N═, ═O, CH₃—O—CH₂—N═, CH₃—O—CH₂—CH₂—N═, CH₃—O—CH₂—CH₂—CH₂—N═, and CH₃—O—CH₂—CH₂—CH₂—CH₂—N═.

20. (canceled)

21. The compound of claim 1 wherein:
X is —NH— and B is ═N—R²: or
X is —NH—, B is ═N—R², and W is —C(O)—; or
X is —NH—. B is ═N—R², and W is —S(O)₂—; or
X is —NH—, B is ═N—R², and R² is alkyl; or
X is —NH—, B is ═N—R², R² is alkyl, and W is —C(O)—; or
X is —NH—, B is ═N—R², R² is alkyl, and W is —S(O)₂—; or
X is —NH═, B is ═N—R², and R² is cycloalkyl; or
X is —NH—, B is ═N—R², R² is cycloalkyl, and W is —C(O)—; or
X is —NH—, B is ═N—R², R² is cycloalkylalkyl, and W is —S(O)₂—; or
X is —NH— and B is ═N-alkyl-OH; or
X is —NH—, B is ═N-alkyl-OH, and W is —C(O)—; or
X is —NH—. B is ═N-alkyl-OH, and W is —S(O)₂—; or
X is —NH—, B is ═N—R², and R² is alkoxyalkyl-; or
X is —NH—, B is ═N—R², R² is alkoxyalkyl-, and W is —C(O)—; or
X is —NH—, B is ═N—R², R² is alkoxyalkyl-, and W is —S(O)₂—; or
X is —N═ and B is alkoxy; or
X is —N═, B is alkoxy, and W is —C(O)—; or
X is —NH—, B is alkoxy, and W is —S(O)₂—; or
X is —N═ and B is heterocycloalkyl; or
X is —N═, B is heterocycloalkyl, and W is —C(O)—; or
X is —N═, B is heterocycloalkyl, and W is —S(O)₂—; or
X is —NH— and B is ═N—O-alkyl; or
X is —NH—, B is ═N—O-alkyl I, and W is —C(O)—; or
X is —NH—, B is ═N—O-alkyl, and W is —S(O)₂—; or
X is —NH—, B is ═N—R², and R² is H; or
X is —NH—, B is ═N—R². R² is H, and W is —C(O)—; or
X is —NH—, B is ═N—R², R² is H, and W is —S(O)₂—.

22-23. (canceled)

24. A compound selected from the group consisting of A9a1 to A9k1, A9n1 to Aq1, A9a to A9u, A9ab, B1-B11 (+)-B11, (−)-B11, B12-B23, C7a to C7f, and D1, or a pharmaceutically acceptable salt thereof.

25-50. (canceled)

51. A pharmaceutical composition comprising:
(a) effective amount of one or more compounds of claim 1, or a pharmaceutically acceptable salt thereof, and an effective amount of one or more other pharmaceutically active ingredients, and a pharmaceutically acceptable carrier; or
(b) effective amount of one or more compounds of claim 1, or a pharmaceutically acceptable salt thereof, and an effective amount of one or more other pharmaceutically active ingredients, and a pharmaceutically acceptable carrier, wherein said other pharmaceutically active ingredients are selected from the group consisting of: BACE inhibitors, muscarinic antagonists, cholinesterase inhibitors; gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic, growth hormone secretagogues; histamine H3 antagonists; AMPA agonists; PDE4 inhibitors. GABA_{A} inverse agonists inhibitors of amyloid aggregation; glycogen synthase kinase beta inhibitors; promoters of alpha secretase activity; PDE-10 inhibitors and cholesterol absorption inhibitors; or
(c) therapeutically effective amount of at least one compound of claim 1, or a pharmaceutically acceptable salt thereof, and at least one pharmaceutically acceptable carrier; or
(b) therapeutically effective amount of at least one compound of claim 1, or a pharmaceutically acceptable salt thereof, and at least one pharmaceutically acceptable carrier, and a therapeutically effective amount of one or more compounds selected from the group consisting of cholinesterase inhibitors. Aβ antibody inhibitors, gamma secretase inhibitors and beta secretase inhibitors.

52-56. (canceled)

57. A method of treating:
(1) a central nervous system disorder comprising:
(a) administering a therapeutically effective amount of at least one compound of claim 1 to a patient in need of such treatment; or
(b) administering a therapeutically effective amount of a pharmaceutical composition comprising a therapeutically effective amount of at least one compound of claim 1, or a pharmaceutically acceptable salt thereof, and at least one pharmaceutically acceptable carrier; or
(c) administering a therapeutically effective amount of a pharmaceutical composition comprising a therapeutically effective amount of at least one compound of claim 1, or a pharmaceutically acceptable salt thereof, and at least one pharmaceutically acceptable carrier, and a therapeutically effective amount of one or more compounds selected from the group consisting of cholinesterase inhibitors, Aβ antibody inhibitors, gamma secretase inhibitors and beta secretase inhibitors, or
(2) modulating gamma secretase activity comprising administering an effective amount of a compound of claim 1 to a patient in need of such treatment; or
(3) inhibiting the deposition of beta amyloid protein comprising administering an effective amount of a compound of claim 1 to a patient in need of such treatment; or
(4) treating one or more neurodegenerative disease comprising administering an effective amount of a compound of claim 1 to a patient in need of such treatment.

58. A method of treating Alzheimers disease comprising:
(a) administering a therapeutically effective amount of at least one compound of claim 1 to a patient in need of such treatment; or
(b) administering a therapeutically effective amount of at least one compound of claim 1, in combination with a therapeutically effective amount of a BACE inhibitor, to a patient in need of such treatment.

59-61. (canceled)

62. A method of:
(1) treating Alzheimer's disease comprising administering one or more compounds of claim 1, in combination with an effective amount of one or more other pharmaceutically active ingredients selected from the group consisting of: BACE inhibitors; muscarinic antagonists; cholinesterase inhibitors; gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors and cholesterol absorption inhibitors, to a patient in need of such treatment; or
(2) treating mild cognitive impairment, comprising administering an effective amount of one or more compounds of claim 1 to a patient in need of treatment; or
(3) treating glaucoma, comprising administering an effective amount of one or more compounds of claim 1 to a patient in need of treatment; or
(4) treating cerebral amyloid angiopathy, comprising administering an effective amount of one or more compounds of claim 1 to a patient in need of treatment; or
(5) treating stroke, comprising administering an effective amount of one or more compounds of claim 1 to a patient in need of treatment; or
(6) treating dementia, comprising administering an effective amount of one or more compounds of claim 1 to a patient in need of treatment; or
(7) treating microgliosis, comprising administering an effective amount of one or more compounds of claim 1 to a patient in need of treatment; or
(8) treating brain inflammation, comprising administering an effective amount of one or more compounds of claim 1 to a patient in need of treatment; or
(9) treating olfactory function loss, comprising administering an effective amount of one or more compounds of claim 1 to a patient in need of treatment; or
(10) treating Downs syndrome comprising administering a therapeutically effective amount of at least one compound of claim 1 to a patient in need of such treatment.

63. (canceled)
